(19) United States
(12) Patent Application Publication
(10) Pub. No.: US 2010/0261758 A1
(43) Pub. Date: Oct. 14, 2010

(54) HETEROCYCLIC AMIDES FOR USE AS PHARMACEUTICALS

(75) Inventors: Luca Arista, Wien (AT); Klemens Hogenauer, Wien (AT); Niko Schmiedeberg, Basel (CH); Gudrun Werner, Wien (AT); Herbert Jaksche, Wien (AT)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG, Basel (CH)

(21) Appl. No.: 12/294,790

(22) PCT Filed: Mar. 28, 2007
(86) PCT No.: PCT/EP2007/002764

§ 371 Date: Sep. 26, 2008

(30) Foreign Application Priority Data
Mar. 28, 2006 (GB) 0606202.0
Sep. 13, 2006 (EP) 06120553.0

(51) Int. Cl. A61K 31/444; C07D 211/82; C07D 213/02; C07D 215/04; C07D 513/04; C07D 233/54; C07D 231/12; C07D 401/02; A61K 31/435; A61K 31/47; A61K 31/426; A61K 31/415; A61K 31/4439; A61P 3/10; A61P 9/00; A61P 3/04

(52) U.S. Cl. 514/332; 546/340; 546/267; 546/152; 548/155; 548/341.5; 548/376.1; 546/277.1; 546/268.4; 514/277; 514/311; 514/368; 514/396; 514/403; 514/339; 514/338

(57) ABSTRACT

Compounds of Formula (I) wherein R₁ is aryl, cyclohexyl or heterocyclyl, or (C₁₋₄)alkyl substituted by aryl, cyclohexyl or heterocyclyl, R₂ is defined heterocyclyl, R₃ is alkyl, aryl, cyclohexyl or heterocyclyl, or (C₁₋₄)alkyl substituted by aryl, cyclohexyl or heterocyclyl, R₄ is H or alkyl, or R₃ and R₄ together with the carbon atom to which they are attached are cycloalkyl fused with aryl, and their use as pharmaceuticals.

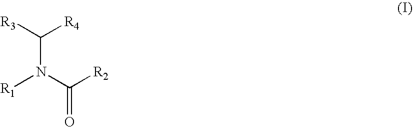

The present invention relates to organic compounds, e.g. compounds which mediate the activity of a specific G protein coupled receptor.

The G protein coupled receptor GPBAR1, e.g. disclosed in WO03051923 (nucleotide sequence SEQ ID NO:1, protein sequence SEQ ID: NO 2), is a member of the G protein-coupled receptor family of polypeptides. The biological properties of such immunomodulatory polypeptides include monocyte/macrophage migration/activation, regulation of dendritic cell differentiation, regulation of lymphocyte activation, proliferation and differentiation regulation of inflammation, regulation of cytokine production and/or release, regulation of pro-inflammatory mediator production and/or release, regulation of immune reaction, GLP (glucagon-like peptide)-1 secretion, insulin secretion, appetite, pancreatic regeneration, pancreatic β cell differentiation, pancreatic β cell growth, insulin resistance, energy expenditure.

Thus, GPBAR1 is indicated to be of interest in relation to methods of treatment of disorders, wherein such biological properties play a causal or contributory role. Such disorders include but are not limited to (chronic) inflammatory diseases, autoimmune diseases, diseases or syndroms in which a significant pathological component is immune suppression, including viral diseases, transplant rejection crisis and other diseases following transplantation, cancer; neurological disorders, such as neurology CNS disorders, cardiovascular disorders, diabetes (type 2), obesity.

Compounds are herewith provided which surprisingly exert agonistic activity on GPBAR1, e.g. thus activating the GPBAR1 function.

In one aspect the present invention provides a compound of formula

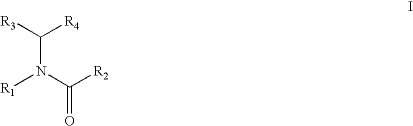

wherein
R₁ is aryl, cyclohexyl or heterocyclyl, or (C₁₋₄)alkyl substituted by aryl, cyclohexyl or heterocyclyl,
preferably R₁ is aryl, aryl(C₁₋₄)alkyl, or heterocyclyl,
wherein aryl is (C₆₋₁₈)aryl, such as (C₆₋₁₂)aryl, e.g. phenyl, naphthalenyl, and wherein aryl may be fused with aliphatic or aromatic heterocyclyl comprising 3 to 12 ring members, e.g. 6, and 1 to 4 heteroatoms selected from N, O, S,
wherein heterocyclyl includes aliphatic or aromatic heterocyclyl, preferably aromatic heterocyclyl, comprising 3 to 12 ring members, such as 5 or 6, and 1 to 4 heteroatoms selected from N, O, S; and wherein heterocycyl may be fused with aryl, such as phenyl or naphthalenyl or may be fused with another heterocyclyl comprising 3 to 12 ring members, such as 5 or 6, and 1 to 4 heteroatoms selected from N, O, S, and
wherein cycloalkyl includes (C₃₋₁₂)cycloalkyl,
R₂ is heterocyclyl selected from the group consisting of
pyridin-4-yl, optionally in the form of an N-oxide, e.g. of formula

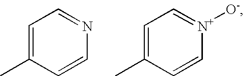

pyridin-4-yl optionally in the form of an N-oxide, substituted one or morefold by (C₁₋₄)alkyl, e.g. methyl, halo(C₁₋₄)alkyl, halogen, e.g. including fluoro, chloro, bromo, such as fluoro, chloro, cyano or di(C₁₋₄)alkylamino,
such as substituted one or morefold by (C₁₋₄)alkyl, e.g. methyl, halogen, cyano or di(C_{1-— 4})alkylamino,
e.g. a group of formula

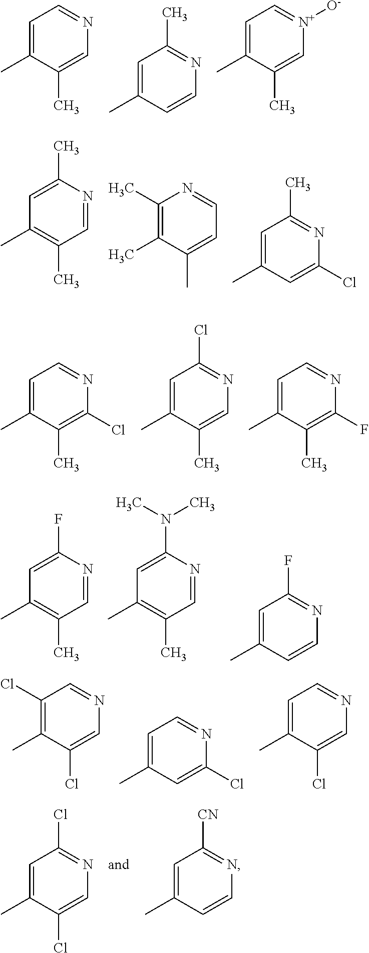

quinolinyl, oxazolyl, isoxazolyl, imidazo[2,1-b]thiazolyl, imidazolyl, pyrazolyl, and benzoimidazolyl,
wherein quinolinyl, oxazolyl, isoxazolyl, imidazo[2,1-b]thiazolyl, imidazolyl, pyrazolyl, or benzoimidazolyl is optionally in the form of an N-oxide and is unsubstituted or substituted, e.g. unsubstituted or substituted one or morefold by (C₁₋₄alkyl, e.g. methyl, halo(C₁₋₄)alkyl, halogen, cyano or di(C₁₋₄)alkylamino, such as substituted by (C₁₋₄)alkyl, e.g. methyl,
such as a group of formula

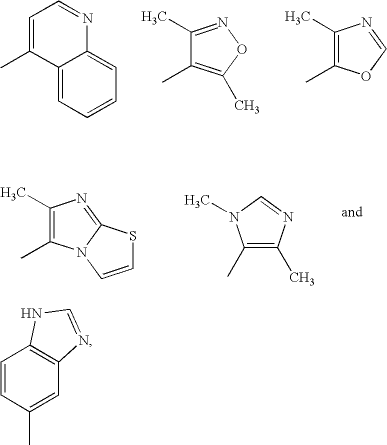

optionally in the form of an N-oxide,
R₃ is alkyl, aryl, cycloalkyl or heterocyclyl, or (C₁₋₄)alkyl substituted by aryl, cycloalkyl or heterocyclyl,
preferably alkyl, aryl, cyclohexyl, heterocyclyl or (C₁₋₄)alkyl substituted by aryl,
wherein alkyl includes (C₁₋₁₂)alkyl, e.g. including straight-chain and branched (C₃₋₁₂)alkyl, wherein aryl includes (C₆₋₁₈)aryl, such as (C₆₋₁₂)aryl, e.g. phenyl, naphtalenyl, biphenylyl, and wherein aryl optionally is fused with aliphatic or aromatic heterocyclyl comprising 3 to 12 ring members, e.g. 5 or 6, and 1 to 4 heteroatoms selected from N, O, S, e.g. N, O,
wherein heterocycyl includes aliphatic or aromatic heterocyclyl comprising 3 to 12 ring members, and 1 to 4 heteroatoms selected from N, O, S and wherein heterocyclyl optionally is fused with another ring (system), e.g. with aryl, such as phenyl or naphthalenyl, or is fused with another heterocyclyl, and
wherein cycloalkyl includes (C₃₋₁₂)cycloalkyl, e.g. (C₃₋₆)cycloalkyl, and
R₄ is H or (C₁₋₄)alkyl; or
R₃ and R₄ together with the carbon atom to which they are attached are cycloalkyl, which cyclyoalkyl is fused with phenyl, such as (C₄₋₈)cycloalkyl fused with phenyl;
wherein aryl, cyclohexyl or heterocyclyl in the meaning of R₁ and R₃ is unsubstituted or one or morefold substituted, e.g. unsubstituted or substituted by one or more, e.g. one or two,
alkyl, e.g. (C₁₋₆)alkyl, alkenyl, e.g. (C₂₋₆)alkenyl,
haloalkyl, e.g. halo(C₁₋₄)alkyl, such as CF₃,
oxo, hydroxy, alkoxy, e.g. including alkoxyalkoxy, such as (C₁₋₄)alkoxy, (C₁₋₄)alkoxy(C₁₋₄)alkoxy, haloalkoxy, e.g. halo(C₁₋₄)alkoxy, such as —OCF₃,
alkylcarbonyloxy, such as (C₁₋₄)alkylcarbonyloxy, aminocarbonyl,
aryl, e.g. (C₆₋₁₂)aryl, such as phenyl,
aryloxy, e.g. (C₆₋₁₂)aryloxy, such as phenoxy,
heterocyclyl including aliphatic and aromatic heterocyclyl having 5 to 6 ring members and 1 to 4 heteroatoms selected from N, O, S, e.g. and wherein heterocyclyl optionally is fused with another ring (system), e.g. with aryl, such as phenyl, or is fused with another heterocyclyl,
cyano, nitro, amino, e,g, unsubstituted and substituted amino, such as di(C₁₋₄)alkylamino, or halogen,
   with the proviso that
compounds of formula

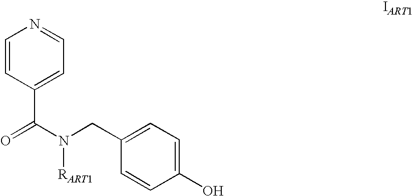

wherein R_{ART1} is selected from phenylethyl, 4-methoxyphenyl, 4-methylphenyl, isobutyl or (furan-2-yl)-methyl,
the compound N-benzyl-N-(2-naphthenyl)-isonicotinamide,
compounds of formula

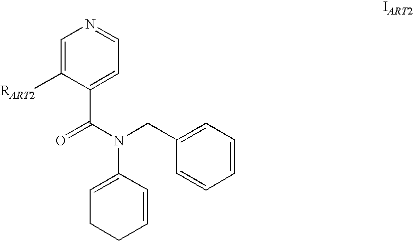

wherein R_{ART2} is iodo or benzyl, and
compounds of formula

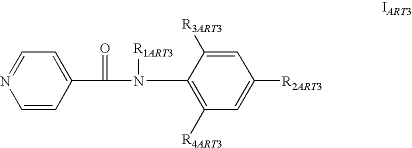

wherein R_{1ART3} is lower alkyl, R_{2ART3} is H, halogen, methyl or nitro, R_{3ART3} is H or halogen and R_{4ART3} is H, halogen, benzoyl or 3-thienoyl,
   are excluded.

Preferably in a compound of formula I

R₁ is (C₆₋₁₂)aryl(C₁₋₄)alkyl, such as phenylethyl,

(C₆₋₁₂)aryl, such as phenyl, naphthalenyl, e.g. naphthalen-1-yl, naphthalen-2-yl,

(C₆₋₁₂)aryl substituted by (C₆₋₁₂)aryl, e.g. biphenylyl,

(C₁₋₄)alkylphenyl, such as (C₁₋₄)alkylphenyl, e.g. tolyl, such as o-tolyl, m-tolyl, p-tolyl, ethylphenyl, e.g. 2-ethylphenyl, propylphenyl, e.g. n-propylphenyl, such as 2-n-propylphenyl, butylphenyl, e.g. tert-butylphenyl, such as 4-tert-butylphenyl,

di(C₁₋₄)alkylphenyl, such as dimethylphenyl, e.g. 2,3-dimethylphenyl, 2,6-dimethylphenyl, (C₁₋₄)alkoxyphenyl, e.g. methoxyphenyl, e.g. 2-methoxyphenyl, 3-methoxyphenyl, phenoxyphenyl, e.g. 4-phenoxyphenyl,

halo(C₁₋₄)alkyl-phenyl, such as halomethylphenyl, e.g. trifluoromethylphenyl, such as 2-trifluoromethylphenyl, 3-trifluoromethylphenyl, 4-trifluoromethylphenyl,

bis-halo(C₁₋₄)alkyl-phenyl, e.g. bis-trifluoromethylphenyl, such as 3,5-bis-trifloromethylphenyl, (halo)((C₁₋₄)alkyl)-phenyl, e.g. (halo)(methyl)-phenyl, such as 2-methyl-4-fluorophenyl, 3-methyl-4-fluorophenyl,

(halo)(halo(C₁₋₄)alkyl)phenyl, such as (halo)(trifluoromethyl)-phenyl, e.g. 3-trifluoromethyl-4-chloro-phenyl, 3-trifluoromethyl-4-fluoro-phenyl, 2-trifluoromethyl-4-fluoro-phenyl, halo(C₁₋₄)alkoxyphenyl, such as halomethoxyphenyl, e.g. trifloromethoxyphenyl, e.g. 2-trifluoromethoxyphenyl,

(halo)(cyano)phenyl, e.g. 2-cyano-4-fluoro-phenyl, 3-cyano-4-fluoro-phenyl, halophenyl, e.g. fluorophenyl, such as 2-fluorophenyl, 3-fluorophenyl, 4-fluorophenyl, chlorophenyl, such as 2-chlorophenyl, 3-chlorophenyl, 4-chlorophenyl, dihalophenyl, such as dichlorophenyl, e.g. 2,4-dichlorophenyl, 3,4-dichlorophenyl, 3,5-dichlorophenyl, (chloro)(fluoro)phenyl, e.g. 2-chloro-4-fluoro-phenyl, 3-chloro-4-fluoro-phenyl, dibromophenyl, e.g. 2,4-dibromophenyl,

cyaonphenyl, such as 2-cyanophenyl, 3-cyanophenyl, 4-cyanophenyl,

nitrophenyl, e.g. 2-nitrophenyl, 3-nitrophenyl, 4-nitrophenyl,

aminocarbonylphenyl, e.g. 2-aminocarbonylphenyl,

5 or 6-membered, e.g. 5 or 6-membered, aromatic or aliphatic, e.g. aromatic, heterocyclyl comprising 1 to 4 heteroatoms, e.g. one or two, selected from N, O, S, e.g. N, such as pyrazolyl, e.g. 1H-pyrazol-3-yl, such as 1-methyl-1H-pyrazol-3-yl, pyridinyl, e.g. pyridine-3-yl, e.g. including cyanopyridinyl, such as 4-cyano-pyridinyl-3-yl, aminocarbonylpyridinyl, such as 4-aminocarbonyl-pyridinyl-3-yl,

or 5 or 6-membered, aromatic or aliphatic, e.g. aromatic, heterocyclyl comprising 1 to 4 heteroatoms selected from N, O, S, such as 6-membered aromatic heterocyclyl comprising 1 or 2 heteroatoms selected from N, O, S, e.g. N, which heterocyclyl is fused with another ring system, e.g. fused with phenyl, such as isoquinolinyl, e.g. isoquinolin-1-yl.

Preferably in a compound of formula I

R₂ is selected from the group consisting of

pyridin-4-yl,

2-methyl-pyridin-4-yl, 3-methyl-pyridin-4-yl,

2,5-dimethyl-pyridin-4-yl, 2,3-dimethyl-pyridin-4-yl, 2,5-dimethyl-pyridin-4-yl,

2-fluoro-pyridin-4-yl, 2-chloro-pyridin-4-yl, 3-chloro-pyridin-4-yl,

2-cyano-pyridin-4-yl,

3,5-dichloro-pyridin-4-yl,

2-chloro-6-methyl-pyridin-4-yl, 2-chloro-3-methyl-pyridin-4-yl, 2-chloro-5-methyl-pyridin-4-yl,

2-fluoro-3-methyl-pyridin-4-yl, 2-fluoro-5-methyl-pyridin-4-yl,

2-amino-pyridin-4-yl, 2-amino-5-methyl-pyridin e.g. including 2-dimethylamino-pyridin-4-yl, and 2-dimethylamino-5-methyl-pyridin,

wherein pyridinyl is optionally in the form of an N-oxide,

quinolinyl, e.g. quinolin-4-yl, e.g. optionally in the form of an N-oxide,

oxazolyl, such as oxazol-5-yl, e.g. including 4-methyl-oxazol-5-yl, e.g. optionally in the form of an N-oxide,

isoxazolyl, such as isoxazol-4-yl, e.g. including 5-methyl-isoxazol-4-yl, 3,5-dimethyl-isoxazol-4-yl, e.g. optionally in the form of an N-oxide,

imidazo[2,1-b]thiazolyl, such as imidazo[2,1-b]thiazol-5-yl, e.g. including 6-methyl-imidazo[2,1-b]thiazol-5-yl), e.g. optionally in the form of an N-oxide,

imidazolyl, such as 3H-imidazol-4-yl, e.g. including 3,5-dimethyl-3H-imidazol-4-yl, e.g. optionally in the form of an N-oxide,

1H-pyrazolyl, such as 1H-pyrazol-4-yl, e.g. including 3,5-dimethyl-1H-pyrazol-4-yl, e.g. optionally in the form of an N-oxide, and

benzoimidazolyl, such as 3H-benzoimidazol-5-yl, e.g. optionally in the form of an N-oxide,

In one preferred aspect of the present invention R₂ is 3-methyl-pyridin-4-yl.

Preferably in a compound of formula I

R₃ is

substituted or unsubstituted (C₁₋₁₂)alkyl, such as (C₁₋₈)alkyl, e.g.

unsubstituted alkyl, such as methyl, isopropyl, 2-methyl-butyl, tert-butyl, 2-ethyl-butyl, 2-methyl-pentyl, or alkyl substituted by aryl, e.g. (C₆₋₁₂)aryl(C₁₋₄)alkyl, e.g. benzyl, (C₃₋₁₂)cycloalkyl, e.g. (C₃₋₈)cycloalkyl, such as cyclopropyl, cyclopentyl, cylohexyl, substituted or unsubstituted (C₆₋₁₂)aryl, such as phenyl,

(C₁₋₆)alkylphenyl, e.g. tolyl, such as o-tolyl, m-tolyl, p-tolyl, ethylphenyl, such as 2-ethylphenyl, isopropylphenyl, such as 4-isopropylphenyl, n-butylphenyl, such as 2-n-butylphenyl,

(C₆₋₁₂)aryl substituted by (C₆₋₁₂)aryl, e.g. biphenylyl,

di(C₁₋₄)alkylphenyl, e.g. dimethylphenyl, such as 2,3-dimethylphenyl, 2,6-dimethylphenyl, halo(C₁₋₄)alkylphenyl, such as trifluoromethylphenyl, e.g. 2-trifluoromethylphenyl, 4-trifluoromethylphenyl,

(C₁₋₄)alkoxyphenyl, such as methoxyphenyl, e.g. 2-methoxyphenyl, isobutoxyphenyl, e.g. 2-isobutoxyphenyl, aminocarbonyl(C₁₋₄alkoxyphenyl, such as aminocarbonylmethoxyphenyl, e.g. 2-aminocarbonylmethoxyphenyl,

(C₁₋₄)alkoxy(C₁₋₄)alkoxyphenyl, such as 2-(ethoxy)-ethoxyphenyl, e.g. 2-[2-(ethoxy)-ethoxy]-phenyl,

di(C₁₋₄alkoxyphenyl, such as dimethoxyphenyl, e.g. 2,6-dimethoxyphenyl,

(C₆₋₁₂)aryloxyphenyl, such as phenoxyphenyl, e.g. 4-phenoxyphenyl,

halo(C₁₋₄alkoxyphenyl, such as trifluoromethoxyphenyl, e.g. 2-trifluoromethoxyphenyl,

(halo)(halo(C₁₋₄)alkyl)phenyl, such as (halo)(trifluoromethyl)-phenyl, e.g. 3-trifluoromethyl-4-chloro-phenyl,

(halo)((C₁₋₄)alkoxy)phenyl, such as (halo)(methoxy)phenyl, e.g. fluoro-methoxy-phenyl, such as difluoro-methoxy-phenyl, e.g. 2,4-difluoro-6-methoxy-phenyl, cyanophenyl, e.g. 2-cyanophenyl, 3-cyanophenyl, 4-cyanophenyl, hydroxyphenyl, e.g. 2-hydroxyphenyl,

(C₁₋₄)alkylcarbonyloxyphenyl, such as methylcarbonyloxyphenyl, aminocarbonylphenyl, halophenyl, e.g. fluorophenyl, such as 3-fluorophenyl, 4-fluorophenyl, chlorophenyl, such as 2-chlorophenyl, 3-chlorophenyl, 4-chlorophenyl, bromophenyl, such as 4-bromophenyl, dihalophenyl, e.g. dichlorophenyl, such as 2,4-dichlorophenyl, 2,6-dichlorophenyl, 3,5-dichlorophenyl,

aminophenyl, such as di(C₁₋₄)alkylaminophenyl, e.g. dimethylaminophenyl, such as 2-dimethylaminocarbonylphenyl,

nitrophenyl, e.g. 3-nitrophenyl, 4-nitrophenyl,

heterocyclylphenyl, wherein heterocyclyl comprises aromatic and aliphatic heterocyclyl,

having 5 to 6 ring members and one to 4 heteroatoms selected from N, O, S, e.g. N, O, such as pyridinylphenyl, e.g. 2-(pyridin-3-yl)-phenyl, 2-(pyridin-4-yl)-phenyl, morpholinophenyl, such as morpholin-4-yl-phenyl, e.g. 2-(morpholin-4-yl)-phenyl, tetrazolyl-phenyl, such as methyl-tetrazolyl-phenyl, e.g. 1-methyl-1H-tetrazol-5-yl-phenyl, such as 3-(1-methyl-1H-tetrazol-5-yl)-phenyl, 2-methyl-2H-tetrazol-5-yl-phenyl, such as 3-(2-methyl-2H-tetrazol-5-yl)-phenyl,

phenyl fused with heterocycyl, wherein heterocyclyl comprises 5 or 6 ring members and 1 to 4 heteroatoms, e.g. 2, selected from N, O, S, e.g. N.O, such as indolyl, e.g. 1H-indol-4-yl, 1H-indol-6-yl, methylindolyl, such as 2-methyl-1H-indol-4-yl, 2,3-dihydro-1H-indolyl, e.g. methyl-2,3-dihydro-1H-indolyl, such as 2,3-dihydro-1H-indol-4-yl, 2-methyl-2,3-dihydro-1H-indol-4-yl, pyridin-carbonyl-2,3-dihydro-1H-indolyl, such as methylpyridine-carbonyl-2,3-dihydro-1H-indolyl, e.g. 3-methyl-pyridine-4-carbonyl)-2,3-dihydro-1H-indol-4-yl, benzo[1,3]dioxolyl, e.g. benzo[1,3]dioxol-4-yl, e.g. including 2,2-difluoro-benzo[1,3]dioxol-4-yl, 2,3-dihydro-benzo[1,4]dioxinyl, such as 2,3-dihydro-benzo[1,4]dioxin-6-yl, 1H-indazol-7-yl, such as 1-methyl-1H-indazol-7-yl, benzo[1,2,5]thiadiazolyl, such as benzo[1,2,5]thiadiazol-4-yl, benzofuranyl, benzofuran-7-yl, methyl-benzofuranyl, e.g. 2-methyl-benzofuran-7-yl, N-quinolin-8-yl, 2-oxo-1,2-dihydro-quinolinyl, such as 2-oxo-1,2-dihydro-quinolin-4-yl, naphthalenyl, e.g. including naphthalen-1-yl, naphthalen-2-yl, (C₁₋₄)alkoxynaphthalenyl, e.g. methoxy-naphthalenyl, such as 3-methoxy-naphthalen-2-yl,

heterocyclyl comprising 5 or 6 ring members and 1 to 4 heteroatoms selected from N, O, S, e.g. arylic hetrocyclyl, e.g. including pyridinyl, e.g. pyridin-2-yl, pyridin-3-yl, methyl-pyridinyl, such as methyl-pyridin-2-yl, e.g. 6-methyl-pyridin-2-yl, morphotinyl-pyridinyl, such as 2-morpholin-4-yl-pyridin-3-yl, furanyl, e.g. furan-2-yl, furan-3-yl, thiophenyl, e.g. thiophen-2-yl, triazolyl, e.g. 2H-[1,2,3]triazol-4-yl), phenyltriazolyl, such as cyanophenyl-triazolyl, e.g. 2-phenyl-2H-[1,2,3]triazol-4-yl, 2-(4-cyano-phenyl)-2H-[1,2,3]triazol-4-yl, oxazolyl, eg. phenyloxyzolyl, chlorophenyloxazolyl, such as oxazol-5-yl, 2-phenyl-oxazol-5-yl, 2-(2-chloro-phenyl)-oxazol-5-yl, thiazolyl, such as thiazol-5-yl, thiazolyl substituted by thiazolyl, such as [2,4′]bithiazolyl-5-yl, methyl-[2,4′]bithiazolyl-5-yl, e.g. 5′-methyl-[2,4′]bithiazolyl-5-yl, pyrrolyl, such as pyrrol-3-yl, phenylpyrrolyl, e.g. 1-phenyl-1H-pyrrol-3-yl,

heterocyclyl comprising 5 or 6 ring members and 1 to 4 heteroatoms selected from N, O, S, which heterocycyl is fused with another ring system,

such as fused with (C₆₋₁₂)aryl, e.g. phenyl, such as quinolinyl, e.g. quinolin-3-yl, aminoquinolinyl, di(C₁₋₄alkylamino-quinolinyl, e.g. dimethylamino-quinolinyl, such as 2-dimethylamino-quinolin-3-yl, 1,2-dihydro-isoquinolinyl, such as 2-methyl-1-oxo-1,2-dihydro-isoquinolin-4-yl, 1H-indol-3-yl, such as cyano-1H-indol-3-yl, e.g. 5-cyano-1H-indol-3-yl, benzothiazolyl, such as benzothiazol-2-yl, oxazolyl, phenyl-oxazolyl, oxochromenyl, such as 4-oxo-4H-chromen-3-yl,

or

such as heterocyclyl fused with another heterocyclyl, e.g. aliphatic or aromatic heterocyclyl, such as aromatic heterocyclyl, wherein heterocyclyl comprising 5 to 6 ring members and 1 to 4 heteroatoms selected from N, O, S, such as Imidazo[2,1-b]thiazolyl, methyl-Imidazo[2,1-b]thiazolyl, e.g. Imidazo[2,1-b]thiazol-5-yl, 6-methyl-imidazo[2,1-b]thiazol-5-yl), imidazo[1,2-a]pyridinyl, e.g. including methyl-imidazo[1,2-a]pyridinyl, such as 2-methyl-imidazo[1,2-a]pyridin-3-yl, or

R₃ and R₄ together with the carbon atom to which they are attached are (C₅₋₈)cycloalkyl which cycloalkyl is fused with phenyl, such as indanyl, e.g. indan-1-yl, tetrahydronaphthalenyl, e.g. (1,2,3,4-tetrahydro-naphthalen-1-yl, tetrahydro-benzocycloheptenyl, such as 6,7,8,9-tetrahydro-5H-benzocyclohepten-5-yl.

Preferably in a compound of formula I R₄ is hydrogen or methyl.

In one preferred embodiment of the present invention R₄ is hydrogen.

In another aspect the present invention provides a compound of formula I, wherein

R₁ is

(C₆₋₁₂)aryl(C₁₋₄)alkyl, phenyl, naphthalenyl, (C₆₋₁₂)aryl substituted by (C₆₋₁₂)aryl,

(C₁₋₈)alkylphenyl, di(C₁₋₄)alkylphenyl, (C₁₋₄alkoxyphenyl, halo(C₁₋₄alkyl-phenyl, bis-halo(C₁₋₄)alkyl-phenyl, (halo)(halo(C₁₋₄)alkyl)phenyl, halo(C₁₋₄)alkoxyphenyl, (halo)(cyano)phenyl halophenyl, dihalophenyl, cyaonphenyl, nitrophenyl, aminocarbonylphenyl, 5 or 6-membered heterocyclyl comprising 1 to 4 heteroatoms, selected from N, O, S, or 5 or 6-membered heterocyclyl comprising 1 to 4 heteroatoms selected from N, O, S, which heterocyclyl is fused with another ring system;

R₂ is selected from the group consisting of

pyridin-4-yl, 2-methyl-pyridin-4-yl, 3-methyl-pyridin-4-yl, 2,5-dimethyl-pyridin-4-yl, 2,3-dimethyl-pyridin-4-yl, 2,5-dimethyl-pyridin-4-yl, 2-fluoro-pyridin-4-yl, 2-chloro-pyridin-4-yl, 3-chloro-pyridin-4-yl, 2-cyano-pyridin-4-yl, 3,5-dichloro-pyridin-4-yl, 2-chloro-6-methyl-pyridin-4-yl, 2-chloro-3-methyl-pyridin-4-yl, 2-chloro-5-methyl-pyridin-4-yl, 2-fluoro-3-methyl-pyridin-4-yl, 2-fluoro-5-methyl-pyridin-4-yl, 2-amino-pyridin-4-yl, 2-amino-5-methyl-pyridin, 2-dimethylamino-pyridin-4-yl, 2-dimethylamino-5-methyl-pyridin,

wherein pyridinyl is optionally in the form of an N-oxide,

quinolinyl, oxazolyl, isoxazolyl, imidazo[2,1-b]thiazolyl, imidazolyl, 1H-pyrazolyl, and

benzoimidazolyl,

optionally in the form of an N-oxide,

R₃ is

unsubstituted (C₁₋₁₂)alkyl, alkyl substituted by (C₆₋₁₂)aryl, (C₃₋₁₂)cycloalkyl, unsubstituted (C₆₋₁₂)aryl, (C₁₋₄)alkylphenyl, (C₆₋₁₂)aryl substituted by (C₆₋₁₂)aryl, di(C₁₋₄)alkylphenyl, halo(C₁₋₄)alkylphenyl, (C₁₋₄)alkoxyphenyl, aminocarbonyl(C₁₋₄)alkoxyphenyl, (C₁₋₄)alkoxy(C₁₋₄)alkoxyphenyl, di(C₁₋₄)alkoxyphenyl, (C₆₋₁₂)aryloxyphenyl, halo(C₁₋₄)alkoxyphenyl, (halo)(halo(C₁₋₄)alkyl)phenyl, (halo)((C₁₋₄)alkoxy)phenyl, cyanophenyl, hydroxyphenyl, (C₁₋₄)alkylcarbonyloxyphenyl, aminocarbonylphenyl, halophenyl, dihalophenyl, aminophenyl, di(C₁₋₄)alkylaminophenyl, nitrophenyl, heterocyclylphenyl, wherein heterocyclyl comprises aromatic and aliphatic heterocyclyl, having 5 to 6 ring members and one to 4 heteroatoms selected from N, O, S, phenyl fused with heterocycyl, wherein heterocyclyl comprises 5 or 6 ring members and 1 to 4 heteroatoms, selected from N, O, S, naphthalenyl, heterocyclyl comprising 5 or 6 ring members and 1 to 4 heteroatoms selected from N, O, S, heterocyclyl comprising 5 or 6 ring members and 1 to 4 heteroatoms selected from N, O, S, which heterocycyl is fused with another ring system,

or R₃ and R₄ together with the carbon atom to which they are attached are (C₅₋₈)cycloalkyl which cycloalkyl is fused with phenyl, and

R₄ is hydrogen or methyl, with the proviso as indicated above.

In a compound of formula I each single defined substituent may be a preferred substituent, e.g. independently of each other substituent defined and each single compound or compound group defined above or below may be a preferred compound group.

In another aspect the present invention provides a compound of formula I, which is selected from the group consisting of
N-(3,5-Dichloro-phenyl)-3-methyl-N-naphthalen-2-ylmethyl-isonicotinamide,
N-(3,5-Dichloro-phenyl)-N-(2-methoxy-benzyl)-3-methyl-isonicotinamide,
3-Methyl-N-phenyl-N-pyridin-3-ylmethyl-isonicotinamide,
N-Naphthalen-2-ylmethyl-1-oxy-N-phenyl-isonicotinamide,
N-(3,5-Dichloro-phenyl)-3-methyl-N-(2-trifluoromethoxy-benzyl)-isonicotinamide,
4-Methyl-oxazole-5-carboxylic acid benzyl-phenyl-amide,
N-Benzyl-N-phenyl-isonicotinamide,
N-Benzyl-N-p-tolyl-isonicotinamide,
N-Benzyl-2-fluoro-N-phenyl-isonicotinamide,
N-Benzyl-3,5-dichloro-N-phenyl-isonicotinamide,
N-Benzyl-2-chloro-N-phenyl-isonicotinamide,
N-Benzyl-2-chloro-6-methyl-N-phenyl-isonicotinamide,
N-Benzyl-3-methyl-N-phenyl-isonicotinamide,
N-Benzyl-3-chloro-N-phenyl-isonicotinamide,
N-Benzyl-2,5-dichloro-N-phenyl-isonicotinamide,
N-Benzyl-2-methyl-N-phenyl-isonicotinamide,
N-Benzyl-2-cyano-N-phenyl-isonicotinamide,
N-Benzyl-N-phenethyl-isonicotinamide,
N-Benzyl-N-(2-fluoromethoxy-phenyl)-isonicotinamide,
N-Benzyl-N-(4-chloro-phenyl)-isonicotinamide,
N-Benzyl-N-(4-tert-butyl-phenyl)-isonicotinamide,
N-Benzyl-N-(4-trifluoromethyl-phenyl)isonicotinamide,
N-Benzyl-N-(4-chloro-3-trifluoromethyl-phenyl)isonicotinamide,
N-Benzyl-N-(2,4-dichloro-phenyl)-isonicotinamide,
N-Benzyl-N-o-tolyl-isonicotinamide,
N-Benzyl-N-(4-fluoro-phenyl)isonicotinamide,
N-Benzyl-N-(3,5-dichloro-phenyl)-isonicotinamide,
N-Benzyl-N-(3-nitro-phenyl)-isonicotinamide,
N-Benzyl-N-(3-methoxy-phenyl)-isonicotinamide,
N-Benzyl-N-(2-trifluoromethyl-phenyl)-isonicotinamide,
N-Benzyl-N-(2-cyano-phenyl)-isonicotinamide,
N-Benzyl-N-(3-chloro-4-fluoro-phenyl)-isonicotinamide,
N-Benzyl-N-naphthalen-2-yl-isonicotinamide,
N-Benzyl-N-naphthalen-1-yl-isonicotinamide,
N-Benzyl-N-(2,3-dimethyl-phenyl)-isonicotinamide,
N-(4-Chloro-benzyl)-N-phenyl-isonicotinamide,
N-(2-Methoxy-benzyl)-N-phenyl-isonicotinamide,
N-(4-Nitro-benzyl)-N-phenyl-isonicotinamide,
N-(4-Methoxy-benzyl)-N-phenyl-isonicotinamide,
N-Phenyl-N-(2-fluoromethyl-benzyl)-isonicotinamide,
N-(3-Chloro-benzyl)-N-phenyl-isonicotinamide,
N-(3-Cyano-benzyl)-N-phenyl-isonicotinamide,
N-(4-Methyl-benzyl)-N-phenyl-isonicotinamide,
N-(2-Methyl-benzyl)-N-phenyl-isonicotinamide,
N-(4-Chloro-benzyl)-3-methyl-N-phenyl-isonicotinamide,
N-(2-Methoxy-benzyl)-3-methyl-N-phenyl-isonicotinamide,
3-Methyl-N-phenyl-N-(4-trifluoromethoxy-benzyl)-isonicotinamide,
3-Methyl-N-phenyl-N-(2-trifluoromethyl-benzyl)-isonicotinamide,
N-(3-Chloro-benzyl)-3-methyl-N-phenyl-isonicotinamide,
N-(3-Cyano-benzyl)-3-methyl-N-phenyl-isonicotinamide,
N-(4-Cyano-benzyl)-3-methyl-N-phenyl-isonicotinamide,
N-(4-Fluoro-benzyl)-3-methyl-N-phenyl-isonicotinamide,
3-Methyl-N-phenyl-N-(4-trifluoromethyl-benzyl-isonicotinamide,
N-Biphenyl-4-ylmethyl-3-methyl-N-phenyl-isonicotinamide,
N-(4-Isopropyl-benzyl)-3-methyl-N-phenyl-isonicotinamide,
3-Methyl-N-(4-phenoxy-benzyl)-N-phenyl-isonicotinamide,
3-Methyl-N-phenyl-N-quinolin-3-ylmethyl)-isonicotinamide,
N-(3,5-Dichloro-benzyl)-3-methyl-N-phenyl-isonicotinamide,
3-Methyl-N-naphthalen-2-ylmethyl-N-phenyl-isonicotinamide,
N-(2,4-Dichloro-benzyl)-3-methyl-N-phenyl-isonicotinamide,
3-Methyl-N-(3-nitro-benzyl)-N-phenyl-isonicotinamide,
N-(2-Chloro-benzyl)-3-methyl-N-phenyl-isonicotinamide,
3-Methyl-N-phenethyl-N-phenyl-isonicotinamide,
3-Methyl-N-(3-methyl-benzyl)-N-phenyl-isonicotinamide,
Quinoline-4-carboxylic acid benzyl-phenyl-amide,
N-Isobutyl-3-methyl-N-phenyl-isonicotinamide,
N-Cyclohexylmethyl-3-methyl-N-phenyl-isonicotinamide,
N-(2,6-Dichloro-benzyl)-3-methyl-N-phenyl-isonicotinamide,
N-(2,6-Dimethyl-benzyl)-3-methyl-N-phenyl-isonicotinamide,
N-(2,6-Dimethoxy-benzyl)-3-methyl-N-phenyl-isonicotinamide,
N-Benzyl-N-(4-fluoro-2-methyl-phenyl)-3-methyl-isonicotinamide,
N-Benzyl-N-(4-fluoro-2-trifluoromethyl-phenyl)-3-methyl-isonicotinamide,
N-Benzyl-N-(2-ethyl-phenyl)-3-methyl-isonicotinamide,
N-Benzyl-3-methyl-N-(2-propyl-phenyl)-isonicotinamide,
N-Benzyl-N-(2-chloro-4-fluoro-phenyl)-3-methyl-isonicotinamide,
Pyridazine-4-carboxylic acid benzyl-phenyl-amide,
3-Methyl-N-phenyl-N-(1-phenyl-ethyl)-isonicotinamide,
N-(3,5-Dichloro-benzyl)-N-(3,5-dichloro-phenyl)-3-methyl-isonicotinamide,
N-Benzyl-N-(4-chloro-phenyl)-3-methyl-isonicotinamide,
N-Benzyl-3-methyl-N-p-tolyl-isonicotinamide,
N-Benzyl-N-(3,4-dichloro-phenyl)-3-methyl-isonicotinamide,
N-Benzyl-N-(3-chloro-phenyl)-3-methyl-isonicotinamide,
N-Benzyl-N-(4-tert-butyl-phenyl)-3-methyl-isonicotinamide,
N-Benzyl-3-methyl-N-(4-trifluoromethyl-phenyl)isonicotinamide,
N-Benzyl-N-(4-chloro-3-trifluoromethyl-phenyl)-3-methyl-isonicotinamide,
N-Benzyl-3-methyl-N-m-tolyl-isonicotinamide,
N-Benzyl-3-methyl-N-(3-trifluoromethyl-phenyl)-isonicotinamide,
N-Benzyl-N-(2,4-dichloro-phenyl)-3-methyl-isonicotinamide,
N-Benzyl-N-(2-chloro-phenyl)-3-methyl-isonicotinamide,
N-Benzyl-3-methyl-N-o-tolyt-isonicotinamide,
N-Benzyl-N-(2-methoxy-phenyl)-3-methyl-isonicotinamide,
N-Benzyl-3-methyl-N-(4-nitro-phenyl)-isonicotinamide,
N-Benzyl-N-(4-fluoro-phenyl)-3-methyl-isonicotinamide,
N-Benzyl-N-(3,5-dichloro-phenyl)-3-methyl-isonicotinamide,
N-Benzyl-3-methyl-N-(3-nitro-phenyl)-isonicotinamide,
N-Benzyl-3-methyl-N-(4-phenoxy-phenyl)-isonicotinamide,
N-Benzyl-N-(3-methoxy-phenyl)-3-methyl-isonicotinamide,
N-Benzyl-3-methyl-N-(2-trifluoromethyl-phenyl)-isonicotinamide,
N-Benzyl-N-(3-fluoro-phenyl)-3-methyl-isonicotinamide,
N-Benzyl-N-(2-fluoro-phenyl)-3-methyl-isonicotinamide,
N-Benzyl-N-(2-cyano-phenyl)-3-methyl-isonicotinamide,
N-Benzyl-N-(3-cyano-phenyl)-3-methyl-isonicotinamide,
N-Benzyl-N-(4-cyano-phenyl)-3-methyl-isonicotinamide,
N-Benzyl-N-(3-chloro-4-fluoro-phenyl)-3-methyl-isonicotinamide,
N-Benzyl-3-methyl-N-naphthalen-2-yl-isonicotinamide,
N-Benzyl-3-methyl-N-naphthalen-1-yl-isonicotinamide,
N-Benzyl-N-(2,3-dimethyl-phenyl)-3-methyl-isonicotinamide,
N-Benzyl-N-(2,6-dimethyl-phenyl)-3-methyl-isonicotinamide,
N-(4-Fluoro-2-methyl-phenyl)-N-isobutyl-3-methyl-isonicotinamide,
N-(4-Fluoro-2-methyl-phenyl)-3-methyl-N-(2-methyl-butyl)-isonicotinamide,
N-(2,2-Dimethyl-propyl)-N-(4-fluoro-2-methyl-phenyl)-3-methyl-isonicotinamide,
N-(2-Ethyl-butyl)-N-(4-fluoro-2-methyl-phenyl)-3-methyl-isonicotinamide,
N-(4-Fluoro-2-methyl-phenyl)-3-methyl-N-2-methyl-pentyl)-isonicotinamide,
N-Cyclopentylmethyl-N-(4-fluoro-2-methyl-phenyl)-3-methyl-isonicotinamide,
N-(3,5-Dichloro-phenyl)-N-isobutyl)-3-methyl-isonicotinamide,
N-(3,5-Dichloro-phenyl)-3-methyl-N-(2-methyl-butyl)-isonicotinamide,
N-(3,5-Dichloro-phenyl)-3-methyl-N-(2-methyl-pentyl)-isonicotinamide,
N-Cyclopentylmethyl-N-(3,5-dichloro-phenyl)-3-methyl-isonicotinamide,
N-(3,5-Dichloro-phenyl)-N-[1-(2-methoxy-phenyl)-ethyl]-3-methyl-isonicotinamide,
N-(3,5-Dichloro-phenyl)-N-(2-ethyl-butyl)-3-methyl-isonicotinamide,
N-Cyclopropylmethyl-N-(3,5-dichloro-phenyl)-3-methyl-isonicotinamide,
N-(2-Chloro-phenyl)-N-(2-methoxy-benzyl)-3-methyl-isonicotinamide,
N-(2-Chloro-benzyl)-N-(2-chloro-phenyl)-3-methyl-isonicotinamide,
N-(2-Chloro-phenyl)-3-methyl-N-(2-methyl-benzyl)-isonicotinamide,
N-(2-Chloro-phenyl)-3-methyl-N-(3-methyl-benzyl)-isonicotinamide,
N-(3,5-Dichloro-phenyl)-3-methyl-N-(3-methyl-benzyl)-isonicotinamide,
N-(3-Chloro-benzyl)-N-(2,6-dimethyl-phenyl)-3-methyl-isonicotinamide,
N-(2,3-Dimethyl-phenyl)-N-(2-methoxy-benzyl)-3-methyl-isonicotinamide,
N-(3-Chloro-benzyl)-N-(2,3-dimethyl-phenyl)-3-methyl-isonicotinamide,
N-(2,3-Dimethyl-phenyl)-3-methyl-N-(3-methyl-benzyl)-isonicotinamide,
N-(2,3-Dimethyl-phenyl)-3-methyl-N-(2-trifluoromethyl-benzyl)-isonicotinamide,
N-(2-Chloro-benzyl)-N-(2,3-dimethyl-phenyl)-3-methyl-isonicotinamide,
3-Methyl-N-(2-trifluoromethoxy-phenyl)-N-(2-trifluoromethyl-benzyl)-isonicotinamide,
N-(2-Chloro-benzyl)-N-(3,4-dichloro-phenyl)-3-methyl-isonicotinamide,
N-(3,4-Dichloro-phenyl)-3-methyl-N-(3-methyl-benzyl)-isonicotinamide,
N-(3-Chloro-benzyl)-N-(3,4-dichloro-phenyl)-3-methyl-isonicotinamide,
N-(3,4-Dichloro-phenyl)-3-methyl-N-(2-methyl-benzyl)-isonicotinamide,
N-(3,4-Dichloro-phenyl)-N-(2-methoxy-benzyl)-3-methyl-isonicotinamide,
N-(2,4-Dichloro-phenyl)-N-(2-methoxy-benzyl)-3-methyl-isonicotinamide,
3,5-Dimethyl-isoxazole-4-carboxylic acid (3,5-dichloro-phenyl)-(2-methoxy-benzyl)amide,
3,5-Dimethyl-isoxazole-4-carboxylic acid phenyl-quinolin-3-ylmethyl-amide,
4-Methyl-oxazole-5-carboxylic acid benzyl-phenyl-amide,
3,5-Dimethyl-isoxazole-4-carboxylic acid benzyl-phenyl-amide,
5-Methyl-isoxazole-4-carboxylic acid benzyl-phenyl-amide,
N-(3,5-Dichloro-phenyl)-N-(2-methoxy-benzyl)-3-methyl-1-oxy-isonicotinamide,
N-(3,5-Dichloro-phenyl)-N-(3-methoxy-naphthalen-2-ylmethyl)-3-methyl-isonicotinamide,
N-(3-Methoxy-naphthalen-2-ylmethyl)-3-methyl-N-phenyl-isonicotinamide,
6-Methyl-imidazo[2,1-b]thiazole-5-carboxylic acid benzyl-phenyl-amide,
N-(3,5-Dichloro-phenyl)-N-[1-(3-methoxy-naphthalen-2-yl)-ethyl]-3-methyl-isonicotinamide,
N-(3,5-Dichloro-phenyl)-3-methyl-N-(2-methyl-benzyl)-isonicotinamide,
N-(3,5-Dichloro-phenyl)-N-[1-(2-methoxy-phenyl)-ethyl]-3-methyl-isonicotinamide, such as N-(3,5-Dichloro-phenyl)-N—[(S)-1-(2-methoxy-phenyl)-ethyl]-3-methyl-isonicotinamide, and N-(3,5-Dichloro-phenyl)-N—[(R)-1-(2-methoxy-phenyl)-ethyl]-3-methyl-isonicotinamide,
N-(3,5-Dichloro-phenyl)-N-[1-(2-methoxy-phenyl)-propyl]-3-methyl-isonicotinamide,
N-(3,5-Dichloro-phenyl)-3-methyl-N-(1-phenyl-ethyl)-isonicotinamide,
N-(3,5-Dichloro-phenyl)-3-methyl-N-(1-o-tolyl-ethyl)-isonicotinamide,
N-(2-Methoxy-benzyl)-3-methyl-N-(2-trifluoromethoxy-phenyl)-isonicotinamide,
N-(3,5-Dichloro-phenyl)-N-(2-dimethylamino-quinolin-3-ylmethyl)-3-methyl-isonicotinamide,
N-(4-Bromo-benzyl)-N-(2,4-dibromo-phenyl)-3-methyl-isonicotinamide,
N-(3,5-Dichloro-phenyl)-N-(2-ethyl-benzyl)-3-methyl-isonicotinamide,
N-(3,5-Dichloro-phenyl)-N-(2,3-dimethyl-benzyl)-3-methyl-isonicotinamide,
6-Methyl-imidazo[2,1-b]thiazole-5-carboxylic acid (3,5-dichloro-phenyl)-(2-methoxy-benzyl)-amide,
6-Methyl-imidazo[2,1-b]thiazole-5-carboxylic acid (3,5-dichloro-phenyl)-[1-(2-methoxy-phenyl)-ethyl]-amide,
N-(3,5-Dichloro-phenyl)-N-[1-(2-ethyl-phenyl)-ethyl]-3-methyl-isonicotinamide,
N-(2,3-Dimethyl-benzyl)-3-methyl-N-(2-trifluoromethoxy-phenyl)-isonicotinamide,
N-(2-Ethyl-benzyl)-3-methyl-N-(2-trifluoromethoxy-phenyl)-isonicotinamide,
N-(3,5-Dichloro-phenyl)-N-[1-(2,3-dimethyl-phenyl)-ethyl]-3-methyl-isonicotinamide,
N-(3,5-Dichloro-phenyl)-3-methyl-N-naphthalen-1-ylmethyl-isonicotinamide,
N-(3,5-Dichloro-phenyl)-3-methyl-N-(1-naphthalen-1-yl-ethyl)-isonicotinamide,
N-(3,5-Dichloro-phenyl)-N-(2-hydroxy-benzyl)-3-methyl-isonicotinamide,
Acetic acid 2-{[(3,5-dichloro-phenyl)-(3-methyl-pyridine-4-carbonyl)-amino]-methyl}-phenyl ester,
N-(3,5-Dichloro-phenyl)-N-[1-(2-hydroxy-phenyl)-ethyl]-3-methyl-isonicotinamide,
N-(2-Cyano-benzyl)-N-(3,5-dichloro-phenyl)-3-methyl-isonicotinamide,
3,5-Dimethyl-3H-imidazole-4-carboxylic acid (3,5-dichloro-phenyl)-(2-methoxy-benzyl)-amide,
3,5-Dimethyl-3H-imidazole-4-carboxylic acid benzyl-phenyl-amide,
N-(3,5-Dichloro-phenyl)-N-[2-(1-ethoxy-ethoxy)-benzyl]-3-methyl-isonicotinamide,
N-(3,5-Dichloro-phenyl)-N-indan-1-yl-3-methyl-isonicotinamide,
N-Indan-1-yl-3-methyl-N-phenyl-isonicotinamide,
3-Methyl-N-phenyl-N-(1,2,3,4-tetrahydro-naphthalen-1-yl)-isonicotinamide,
3-Methyl-N-phenyl-N-(6,7,8,9-tetrahydro-5H-benzocyclohepten-5-yl)-isonicotinamide,
N-Biphenyl-2-yl-N-ethyl-3-methyl-isonicotinamide,
N-(2-Carbamoylmethoxy-benzyl)-N-(3,5-dichloro-phenyl)-3-methyl-isonicotinamide,
N-(3,5-Dichloro-phenyl)-N-(2-isobutoxy-benzyl)-3-methyl-isonicotinamide,
N-Biphenyl-2-yl-N-isobutyl-3-methyl-isonicotinamide,
N-(3,5-Dichloro-phenyl)-3-methyl-N-(2-pyridin-3-yl-benzyl)-isonicotinamide,
N-Biphenyl-2-ylmethyl-N-(3,5-dichloro-phenyl)-3-methyl-isonicotinamide,
N-(3,5-Dichloro-phenyl)-3-methyl-N-(2-pyridin-4-yl-benzyl)-isonicotinamide,
N-(3,5-Dichloro-phenyl)-3-methyl-N-(2-pyridin-2-yl-benzyl)-isonicotinamide,
N-(2-Butyl-benzyl)-N-(3,5-dichloro-phenyl)-3-methyl-isonicotinamide,
N-Benzyl-2-dimethylamino-5-methyl-N-phenyl-isonicotinamide,
N-(3,5-Dichloro-phenyl)-N-ethyl-3-methyl-isonicotinamide,
N-(3,5-Dichloro-phenyl)-N-(2-dimethylamino-benzyl)-3-methyl-isonicotinamide,
N-(3,5-Dichloro-phenyl)-3-methyl-N-(2-morpholin-4-yl-benzyl)-isonicotinamide,
N-(3,5-Dichloro-phenyl)-N-(2,2-difluoro-benzo[1,3]dioxol-4-ylmethyl)-3-methyl-isonicotinamide,
3,5-Dimethyl-1H-pyrazole-4-carboxylic acid benzyl-phenyl-amide,
3,5-Dimethyl-1H-pyrazole-4-carboxylic acid (3,5-dichloro-phenyl)-(2-methoxy-benzyl)amide,
N-Benzyl-2-chloro-3-methyl-N-phenyl-isonicotinamide,
N-Benzyl-2-chloro-5-methyl-N-phenyl-isonicotinamide,
N-Benzyl-2,5-dimethyl-N-phenyl-isonicotinamide,
N-Benzyl-2,3-dimethyl-N-phenyl-isonicotinamide,
N-Benzyl-2-fluoro-3-methyl-N-phenyl-isonicotinamide,
N-Benzyl-2-fluoro-5-methyl-N-phenyl-isonicotinamide,
N-(4-Fluoro-2-methyl-phenyl)-N-(2-methoxy-benzyl)-3-methyl-isonicotinamide,
N-(4-Fluoro-3-methyl-phenyl)-N-(2-methoxy-benzyl)-3-methyl-isonicotinamide,
N-(2-Chloro-4-fluoro-phenyl)-N-(2-methoxy-benzyl)-3-methyl-isonicotinamide,
N-(4-Fluoro-3-trifluoromethyl-phenyl)-N-(2-methoxy-benzyl)-3-methyl-isonicotinamide,
N-(3-Cyano-4-fluoro-phenyl)-N-(2-methoxy-benzyl)-3-methyl-isonicotinamide,
N-(3,5-Dichloro-phenyl)-N-furan-3-ylmethyl-3-methyl-isonicotinamide,
N-(3,5-Dichloro-phenyl)-N-(4-fluoro-benzyl)-3-methyl-isonicotinamide,
N-(3,5-Dichloro-phenyl)-N-furan-2-ylmethyl-3-methyl-isonicotinamide,
N-(3,5-Dichloro-phenyl)-3-methyl-N-thiophen-2-ylmethyl-isonicotinamide,
N-(3,5-Dichloro-phenyl)-N-(1H-indol-4-ylmethyl)-3-methyl-isonicotinamide,
N-(3,5-Dichloro-phenyl)-N-(2,3-dihydro-benzo[1,4]dioxin-6-ylmethyl)-3-methyl-sonicotinamide,
N-(3,5-Dichloro-phenyl)-3-methyl-N-(6-methyl-pyridin-2-ylmethyl)-isonicotinamide,
N-(3,5-Dichloro-phenyl)-N-(1H-indol-5-ylmethyl)-3-methyl-isonicotinamide,
N-(3,5-Dichloro-phenyl)-N-(1H-indol-6-ylmethyl)-3-methyl-isonicotinamide,
N-(5-Cyano-1H-indol-3-ylmethyl)-N-(3,5-dichloro-phenyl)-3-methyl-isonicotinamide,
N-(3,5-Dichloro-phenyl)-3-methyl-N-(1-methyl-1H-indazol-7-ylmethyl)-isonicotinamide,
N-(3,5-Dichloro-phenyl)-3-methyl-N-[3-(1-methyl-1H-tetrazol-5-yl)-benzyl]-isonicotinamide,
N-(3,5-Dichloro-phenyl)-3-methyl-N-[3-(2-methyl-2H-tetrazol-5-yl)-benzyl]-isonicotinamide,
N-[2-(4-Cyano-phenyl)-2H-[1,2,3]triazol-4-ylmethyl]-N-(3,5-dichloro-phenyl)-3-methyl-isonicotinamide,
N-(3,5-Dichloro-phenyl)-3-methyl-N-(4-oxo-4H-chromen-3-ylmethyl)-isonicotinamide,
N-Benzothiazol-2-yl-methyl-N-(3,5-dichloro-phenyl)-3-methyl-isonicotinamide,
N-[2-(2-Chloro-phenyl)-oxazol-5-yl-methyl]-N-(3,5-dichloro-phenyl)-3-methyl-isonicotinamide,
N-(3,5-Dichloro-phenyl)-3-methyl-N-(2-methyl-1H-indol-4-ylmethyl)-isonicotinamide,
N-(3,5-Dichloro-phenyl)-N-(2,4-difluoro-6-methoxy-benzyl)-3-methyl-isonicotinamide,
N-(3,5-Dichloro-phenyl)-3-methyl-N-(6-methyl-imidazo[2,1-b]thiazol-5-ylmethyl)-sonicotinamide,
N-(3,5-Dichloro-phenyl)-3-methyl-N-(2′-methyl-[2,4′]bithiazolyl-5-ylmethyl)-isonicotinamide,
N-Benzo[1,2,5]thiadiazol-4-ylmethyl-N-(3,5-dichloro-phenyl)-3-methyl-isonicotinamide,
N-(3,5-Dichloro-phenyl)-3-methyl-N-(2-methyl-benzofuran-7-ylmethyl)-isonicotinamide,
N-(3,5-Dichloro-phenyl)-3-methyl-N-(2-methyl-imidazo[1,2-a]pyridin-3-ylmethyl)-isonicotinamide,
N-(3,5-Dichloro-phenyl)-3-methyl-N-(2-methyl-1-oxo-1,2-dihydro-isoquinolin-4-ylmethyl)-isonicotinamide,
N-(3,5-Dichloro-phenyl)-3-methyl-N-quinolin-8-ylmethyl-isonicotinamide,
N-(3,5-Dichloro-phenyl)-3-methyl-N-(1-phenyl-1H-pyrrol-3-yl-methyl)-isonicotinamide,
N-(3,5-Dichloro-phenyl)-3-methyl-N-(2-morpholin-4-yl-pyridin-3-ylmethyl)-isonicotinamide,
N-(3-Chloro-5-trifluoromethyl-benzyl)-3-methyl-N-(1-methyl-1H-pyrazol-3-yl)-isonicotinamide,
N-(4-tert-Butyl-phenyl)-3-methyl-N-naphthalen-1-ylmethyl-isonicotinamide,
N-Isoquinolin-1-yl-3-methyl-N-(1-phenyl-ethyl)isonicotinamide, such as N-Isoquinolin-1-yl-3-methyl-N—((R)-1-phenyl-ethyl)-isonicotinamide,
N-(3,5-Dichloro-phenyl)-3-methyl-N-(2-oxo-1,2-dihydro-quinolin-4-ylmethyl)-isonicotinamide,
N-(2-Cyano-4-fluoro-phenyl)-N-(2-methoxy-benzyl)-3-methyl-isonicotinamide,
N-(3,5-Dichloro-phenyl)-3-methyl-N-(2-methyl-2,3-dihydro-1H-indol-4-ylmethyl)-isonicotinamide,
N-(3,5-Dichloro-phenyl)-3-methyl-N-[2-methyl-1-(3-methyl-pyridine-4-carbonyl)-2,3-dihydro-1H-indol-4-ylmethyl]-isonicotinamide,
N-Biphenyl-2-yl-N-(4-fluoro-benzyl)-3-methyl-isonicotinamide,
N-(6-Cyano-pyridin-3-yl)-N-(4-fluoro-benzyl)-3-methyl-isonicotinamide,
N-(2,3-Dichloro-phenyl)-3-methyl-N-pyridin-2-ylmethyl-isonicotinamide,
N-(4-Fluoro-benzyl)-3-methyl-N-(6-aminocarbonyl-pyridin-3-yl)-isonicotinamide,
N-(3,5-Bis-trifluoromethyl-phenyl)-N-(2,6-dichloro-benzyl)-3-methyl-isnicotinamide,
N-(2-Carbamoyl-phenyl)-N-(4-fluoro-benzyl)-3-methyl-isonicotinamide,
N-(2,6-Dimethyl-phenyl)-3-methyl-N-pyridin-3-ylmethyl-isonicotinamide,
3-Methyl-N-(1-pyridin-2-yl-ethyl)-N-(4-trifluoromethyl-phenyl)-isonicotinamide,
1H-Benzoimidazole-5-carboxylic add (3,5-dichloro-phenyl)-(2-methoxy-benzyl)-amide,
N-(4-Fluoro-2-methyl-phenyl)-3-methyl-N-(2-methyl-1H-indol-4-ylmethyl)-isonicotinamide,
N-(2-Chloro-4-fluoro-phenyl)-3-methyl-N-(2-methyl-1H-indol-4-ylmethyl)-isonicotinamide,
N-(4-Fluoro-2-methyl-phenyl)-3-methyl-N-(6-methyl-imidazo[2,1-b]thiazol-5-ylmethyl)-isonicotinamide,
N-(2-Chloro-4-fluoro-phenyl)-3-methyl-N-(6-methyl-imidazo[2,1-b]thiazol-5-ylmethyl)-isonicotinamide,
N-(3,4-Dichloro-phenyl)-3-methyl-N-(6-methyl-imidazo[2,1-b]thiazol-5-ylmethyl)-isonicotinamide,
N-(4-Fluoro-3-trifluoromethyl-phenyl)-3-methyl-N-(6-methyl-imidazo[2,1-b]thiazol-5-ylmethyl)-isonicotinamide,
N-(4-Fluoro-2-methyl-phenyl)-2,3-dimethyl-N-(2-methyl-1H-indol-4-ylmethyl)-isonicotinamide,
N-(4-Fluoro-2-methyl-phenyl)-2,5-dimethyl-N-(2-methyl-1H-indol-4-ylmethyl)-isonicotinamide,
N-(3-Cyano-4-fluoro-phenyl)-3-methyl-N-(2-methyl-1H-indol-4-ylmethyl)-isonicotinamide,
3-Methyl-N-(2-methyl-1H-indol-4-ylmethyl)-N-(3-trifluoromethyl-phenyl)-isonicotinamide,
3-Methyl-N-(2-methyl-1H-indol-4-ylmethyl)-N-(3-trifluoromethyl-phenyl)-isonicotinamide

N-(2,4-Dichloro-phenyl)-3-methyl-N-(2-methyl-1H-indol-4-ylmethyl)-isonicotinamide,
N-(4-Chloro-phenyl)-3-methyl-N-(2-methyl-1H-indol-4-ylmethyl)-isonicotinamide, and
N-(4-Cyano-phenyl)-3-methyl-N-(2-methyl-1H-indol-4-ylmethyl)-isonicotinamide,
   e.g. which are compounds as indicated in TABLE 1 of the examples with the exception of the compounds of examples 158 and 159.

Any group indicated or defined herein may be unsubstituted or substituted, e.g. one or morefold, e.g. such as indicated herein. Substituents include groups which are conventional in organic chemistry, e.g. such as indicated herein.

Compounds provided by the present invention are hereinafter designated as “compound(s) of (according to) the present invention”. A compound of the present invention includes a compound in any form, e.g. in free form, in the form of a salt, in the form of a solvate and in the form of a salt and a solvate.

In another aspect the present invention provides a compound of the present invention in the form of a salt.

Such salts include preferably pharmaceutically acceptable salts, although pharmaceutically unacceptable salts are included, e.g. for preparation/isolation/purification purposes.

A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of isomers and mixtures thereof; e.g. optical isomers, diastereoisomers, cis/trans conformers. A compound of the present invention may e.g. contain asymmetric carbon atoms and may thus exist in the form of enantiomers or diastereoisomers and mixtures thereof, e.g. in the form of a racemat. A compound of the present invention may be present in the (R)-, (S)- or (R,S)-configuration preferably in the (R)- or (S)-configuration regarding specified positions in the compound. E.g. in a compound of formula I, wherein R₃ is branched (C₃₋₈)alkyl or substituted alkyl, or substituted cycloalkyl, e.g. or in a compound of formula I, wherein R₄ is alkyl, asymmetric carbon atoms may exist, e.g. the carbon atom to which R₃ and R₄ are attached may be asymmetric, and compounds comprising an asymmetric carbon atom may be in the (R)-, -(S)- or (R/S)-form regarding the position of an asymmetric carbon atom.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of the present invention, where tautomers can exist.

In another aspect the present invention provides a process for the production of a compound of the present invention, e.g. of formula I, comprising reacting a compound of formula

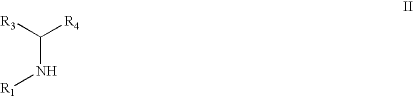

wherein R₁, R₃ and R₄ are as defined above, with a compound of formula

HOOC—R₂ III

wherein R₂ is as defined above, e.g. wherein a compound of formula III is in an activated form, e.g. reacted with 1-chloro-N,N,2-trimethyl-1-propenylamine,
in the presence of an amine, e.g. triethylamine, and isolating a compound of formula I obtained from the reaction mixture.

A compound of formula II wherein R₄ is hydrogen may be e.g. obtained by reacting a compound of formula

R₃CHO IV

wherein R₃ is as defined above, with a compound of formula

R₁—NH₂ V

wherein R₁ is as defined above, in the presence of a reducing agent, such as sodium triacetoxyborohydride, and isolating a compound of formula II obtained from the reaction mixture.

A compound of formula II wherein R₄ is alkyl may be e.g. obtained by reacting a compound of formula

R₁—NH₂ V

wherein R₁ is as defined above, with a compound of formula

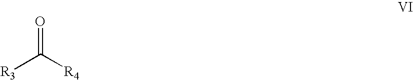

wherein R₃ is as defined above and R₄ is alkyl, in the presence of an amine, e.g. triethylamine, followed by treating the reaction mixture obtained with titanium tetrachloride and sodium cyanoborohydride; and isolating a compound of formula II wherein R₄ is alkyl, obtained from the reaction mixture.

In an intermediate of formula II, III, IV, V or VI (starting materials), functional groups, if present, optionally may be in protected form or in the form of a salt, if a salt-forming group is present. Protecting groups, optionally present, may be removed at an appropriate stage, e.g. according, e.g. analogously, to a method as conventional

A compound of formula I thus obtained may be converted into another compound of formula I, e.g. or a compound of formula I obtained in free form may be converted into a salt of a compound of formula I and vice versa.

The above reaction between a compound of formula II and a compound of formula III is an acylation reaction rand may be carried out as appropriate, e.g. according, e.g. analogously, to a method as conventional.

Intermediates (starting materials) of formula II, III, IV, V and VI are known or may be prepared according, e.g. analogously, to a method as conventional or as described herein. Any compound described herein, e.g. a compound of the present invention and a compound for the treatment of disorders which are mediated by GPBAR1 or for the manufacture of a medicament thereof, and intermediates of formula II, III, IV, V and VI may be prepared as appropriate, e.g. according, e.g. analogously, to a method as conventional, e.g. or as specified herein.

The compounds of the present invention and compounds of formula I, wherein R₃ is as defined above and additionally denotes (C₂₋₄)alkynyl-phenyl, e.g. including the compounds N-(2-ethynyl-benzyl)-3-methyl-N-phenyl-isonicotinamide (compound of example 158 in TABLE 1) and N-(4-ethynyl-benzyl)-3-methyl-N-phenyl-isonicotinamide (compound of example 159 in TABLE 1), e.g. in free form or in the form of a salt, e.g. optionally in the form of a solvate, exhibit pharmacological activity and are therefore useful as pharmaceuticals.

The compounds of the present invention and compounds of formula I, wherein R₃ is as defined above and additionally denotes (C₂₋₄)alkynyl-phenyl, e.g. including the compounds N-(2-ethynyl-benzyl)-3-methyl-N-phenyl-isonicotinamide (compound of example 158 in TABLE 1) and N-(4-ethynyl-benzyl)-3-methyl-N-phenyl-isonicotinamide (compound of example 159 in TABLE 1), e.g. in free form or in the form of a salt, e.g. optionally in the form of a solvate, are herein also designated as “specific GPBAR1 compound(s) of (according to) the present invention”.

The specific GPBAR1 compounds of the present invention show agonistic activity on GPBAR1, and are prone for the treatment of disorders which are mediated by, e.g. dysfunctional, e.g. insufficient, GPBAR1 activity.

Pharmaceutical activity of the specific GPBAR1 compounds of the present invention e.g. may be shown in the cAMP Assay, e.g. GPBAR1 is a G_{αs}-coupled GPCR and ligands induce the formation of cAMP in cells expressing GPBAR1.

### cAMP Assay

### ABBREVIATIONS

cAMP Cyclic adenosine 3′,5′-monophosphate

EC₅₀ Agonist concentration that produces 50% of the maximal effect

GPCR G protein-coupled receptor

G_{αs} Adenylate cyclase-stimulating G protein

GFP Green fluorescent protein

HBSS Hanks' Balanced Salt Solution

HTRF Homogeneous Time-Resolved Fluorescence

FRET Fluorescence Resonance Energy Transfer

IBMX 3-isobutyl-1-methylxanthine

RT Room Temperature

The human lymphoblastoid cell line Jurkat is transduced with a murine leukaemia based replication-defective retroviral vector construct to mediate stable expression of the ORP9651 cDNA. Briefly, the cDNA of the human GPBAR1 gene is cloned into the retroviral expression vector pMXpie, which contains an IRES (internal ribosomal entry site)-GFP expression cassette and a puromycin resistance gene. Phoenix™-Ampho packaging cells are transfected using LipofectAMINE (Invitrogen) as described by the manufacturer. At 24 h after transfection, supernatants containing retrovirus are harvested and filtered (0.2 μm). For retroviral infection of Jurkat cell lines, 2×10⁶ cells are incubated with virus-containing supernatants supplemented with 10 μg/ml of Polybrene (Sigma). After 48 h of culture, Jurkat cells expressing high levels of GFP are collected by fluorescence-activated cell sorting and subsequently cultured in AIM-V serum-free medium (GIBCO BRL) containing 1 μg/ml puromycin, 1 IE/ml penicillin and 1 μg/ml streptomycin. Expression of the GPBAR1 gene is verified by RT-PCR.

Experiments to determine changes in cAMP after compound addition to Jurkat cells expressing GPBAR1 are performed with the HTRF kit from CIS Bio International (Bagnols sur Ceze, France). The method is based on a competitive immunoassay between native CAMP produced by cells and added cAMP labeled with XL665 and is performed according to instructions by the manufacturer in 384 well black FIA plates (Greiner) and a final volume of 20 μl per well. Briefly, assay plates containing 5 μl of cell suspension, adjusted to 1×10⁶ cells per ml HBSS (GIBCO BRL) containing 1 mM IBMX (Sigma), and 5 μl of compound dilution are incubated at RT for 30 minutes in a humidified box to stimulate cAMP production. The total cAMP concentration in cells is analyzed by adding 5 μl cAMP-XL655 and 5 μl of anti-cAMP-Cryptate antibody solution, both pre-diluted 1:20 in conjugation/lysis buffer, as supplied by the manufacturer. After another incubation for 1 hour in a humidified box FRET, measurements are performed with the PHERAstar (BMT Labtech) plate reader (excitation 337 nm, emission 620 and 665 nm). Data are calculated from intensities of emitted light filtered at two wavelengths L1 (665 nM) and L2 (620 nM) as the ratio L1/L2 and normalised by ΔF=[(sample ratio−negative ratio)/negative ratio]×100.

The selectivity of compounds for GPBAR1 is determined in cAMP assays using a Jurkat control cell line generated by transduction of empty pMXpie vector following exactly the same protocol as described above. All compounds are inactive up to a concentration of 20 μM in that cell line.

The specific GPBAR1 compounds of the present invention exhibit EC₅₀ values in the cAMP Assay as described above, from the low nanomolar range up to low micromolar range, e.g. 0.3 nM up to 5 μM. The compounds of the present are therefore prone to be useful for the treatment of disorders mediated by GPBAR1 activity, e.g. insufficient GPBAR1 activity.

Disorders as used herein include diseases.

Disorders mediated by GPBAR1 activity which are prone to be successfully treated with GPBAR1 agonists, e.g. with a specific GBPAR1 activating compound of the present invention, include disorders, wherein the activity of GPBAR1 play a causal or contributory role, such as immune responses initiated by dendritic cells (DCs), monocytes or lymphocytes.

Such disorders e.g. include, but are nit limited to
disorders associated with inflammation
e.g. including (chronic) inflammatory disorders, disorders related with the inflammation of the bronchi, e.g. including bronchitis, cervix, e.g. including cervicitis, conjunctiva, e.g. conjunctivitis, esophagus, e.g. esophagitis, heart muscle, e.g. myocarditis, rectum, e.g. proctitis, sclera, e.g. scleritis, gums, involving bone, pulmonary inflammation (alveolitis), airways, e.g. asthma, such as bronchial asthma, acute respiratory distress syndrome (ARDS), inflammatory skin disorders such as contact hypersensitivity, atopic dermatitis; fibrotic disease (e.g., pulmonary fibrosis), encephilitis, inflammatory osteolysis,
disorders associated with conditions of the immune system,
immune, such as autoimmune disorders e.g. including Graves' disease, Hashimoto's disease (chronic thyroiditis), multiple sclerosis, rheumatoid arthritis, arthritis, gout, osteoarthritis, scleroderma, lupus syndromes, systemic lupus erytomatosis, Sjoegren's syndrome, psoriasis, inflammatory bowel disease, including Crohn's disease, colitis, e.g. ulcerative colitis; sepsis, septic shock, autoimmune hemolytic anemia (AHA), autoantibody triggered urticaria, pemphigus, nephritis, glomerulonephritis, Goodpastur syndrom, ankylosing spondylitis, Reiter's syndrome, polymyositis, dermatomyositis, cytokine-mediated toxicity, interleukin-2 toxicity, alopecia greata, uveitis, lichen planus, bullous pemphigoid, myasthenia gravis, type I diabetes mellitus, immune-mediated infertility such as premature ovarian failure, polyglandular failure, hypothyroidism, pemphigus vulgaris, pemphigus I-oliaceus, paraneoplastic pemphigus, autoimmune hepatitis including that associated with hepatitis B virus (HBV) and hepatitis C virus (HCV), Addison's disease, autoimmune skin diseases, such as psoriasis, dermatitis herpetiformis, epidermolysis bullosa, linear IgA bullous dermatosis, epidermolysis bullosa acquisita, chronic bullous disease of childhood, pernicious anemia, hemolytic anemia, vitiligo, type I, type II and type III autoimmune polyglandular syndromes, Autoimmune Hypoparathyroidism, Autoimmune Hypophysitis, Autoimmune Oophoritis, Autoimmune Orchitis, pemphigoid gestationis, cicatricial pemphigoid, mixed essential cryoglobulinemia, immune thrombocytopenic purpura, Goodpasture's syndrome, autoimmune neutropenia, Eaton-Lambert myasthenic syndrome, stiff-man syndrome, encephalomyelitis, acute disseminated encephalomyelitis, Guillain-Barre syndrome, cerebellar degeneration, retinopathy, primary biliary sclerosis, sclerosing cholangitis autoimmune hepatitis, gluten-sensitive enteropathy, reactive arthritides, polymyositis/dermatomyositis, mixed connective tissue disease, Bechet's syndrome, polyarteritis nodosa allergic anguitis and granulomatosis (Churg-Strauss disease), polyangiitis overlap syndrome (hypersensitivity) vasculitis, Wegener's granulomatosis, temporal arteritis Kawasaki's disease, sarcoidosis, cryopathies, Celiac disease,
disorders associated with cytokine-mediated toxicity,
e.g. including interleukin-2 toxicity,
disorders associated with the bone,
e.g. including osteoporosis, osteoarthritis,
disorders associated with the brain and the nerves,
neurodegenerative disorders, e.g. including disorders of the central nervous system as well as disorders of the peripheral nervous system, e.g. CNS disorders including central nervous infections, brain injuries, cerebrovascular disorders and their consequences, Parkinson's disease, corticobasal degeneration, motor neuron disease, dementia including ALS, multiple sclerosis, traumatic disorders, including trauma and inflammatory consequences of trauma, traumatic brain injury, stroke, post-stroke, post-traumatic brain injury,
small-vessel cerebrovascular disease, eating disorders; further dementias, e.g. including Alzheimer's disease, vascular dementia, dementia with Lewy-bodies, frontotemporal dementia and Parkinsonism linked to chromosome 17, frontotemporal dementias, including Pick's disease, progressive nuclear palsy, corticobasal degeneration, Huntington's disease, thalamic degeneration, Creutzfeld Jakob dementia, HIV dementia, schizophrenia with dementia, Korsakoffs psychosis,
cognitive-related disorders, such as mild cognitive impairment, age-associated memory impairment, age-related cognitive decline, vascular cognitive impairment, attention deficit disorders, attention deficit hyperactivity disorders, and memory disturbances in children with learning disabilities; conditions associated with the hypothalamic-pituitary-adrenal axis,
neuronal disorders, e,g, including neuronal migration disorders, hypotonia (reduced muscle tone), muscle weakness, seizures, developmental delay (physical or mental development difficulty), mental retardation, growth failure, feeding difficulties, lymphedema, microcephaly, symptoms affecting the head and the brain, motor dysfunction;
disorders associated with the eye,
e.g. including uveoritinitis, vitreoretinopathy, corneal disease, iritis, iridocyclitis, cateracts, uveitis, diabetic retinopathy, retinitis pigmentosa, conjunctivits, keratitis,
disorders associated with the gastrointestinal tract
e.g. including colitis, inflammatory bowel disease, Crohn's disease, ulcerative colitis, peptic ulceration, gastritis, oseophagitis,
disorders associated with the heart and vascular conditions
e.g. including cardiovascular disorders, e.g. including cardiac failure, cardiac infarction, cardiac hypertrophy, heart failure, e.g. including all forms of heart pumping failures such as high-output and low-output, acute and chronic, right sided or left-sided, systolic or diastolic, independent of the underlying cause; myocardial infarction (MI), MI prophylaxis (primary and secondary prevention), acute treatment of MI, prevention of complications; heart disorders, proliferative vascular disorders, vasculitides, polyarteritis nodosa, inflammatory consequences of ischemia, ischemic heart disease, myocardial infarction, stroke, peripheral vascular disease, pulmonary hypertension,
ischemic disorders, e.g. including myocardial ischemia, e.g. stable angina, unstable angina, angina pectoris, bronchitis; asymptomatic arrhythmias such as all forms of atrial and ventricular tachyarrhythmias, atrial tachycardia, atrial flutter, atrial fibrillation, atrio-ventricular reentrant tachycardia; preexitation syndrome, ventricular tachycardia, ventricular flutter, ventricular fibrillation, bradycardic forms of arrhythmias; arrhythmia, chronic obstructive pulmonary disease,
hypertension, such as systolic or diastolic high blood pressure, e.g essential and secondary hypertension, e.g. including hypertensive vascular disorders, such as primary as well as all kinds of secondary arterial hypertension, renal, endocrine, neurogenic and others; peripheral vascular disorders in which arterial and/or venous flow is reduced resulting in an imbalance between blood supply and tissue oxygen demand, e.g. including artherosclerosis, chronic peripheral arterial occlusive disease (PAOD), acute arterial thrombosis and embolism, inflammatory vascular disorders, Raynaud's phenomenon and venous disorders; atherosclerosis, a disease in which the vessel wall is remodeled, e.g. including accumulation of cells, both smooth muscle cells and monocyte/macrophage inflammatory cells, in the intima of the vessel wall;
hypotension,
disorders associated with the liver and the kidneys,
e.g. including renal disorders, kidney disorders, e.g. acute kidney failure, acute renal disease, liver disorders, e.g. cirrhosis, hepatitis, liver failure, cholestasis, acute/chronic hepatitis, sclerosing cholangitis, primary billiary cirrhosis, acute/chronic interstitial/glomerulonephritis, granulomatous diseases,
disorders associated with stomach or pancreas conditions
e.g. including stomach disorders, e.g. gastric ulcer, gastrointestinal ulcer, pancreatic disorders, pancreatic fatigue,
disorders associated with the respiratory tract and lung
e.g. including pulmonary disorders, chronic pulmonary disease, acute (adult) respiratory distress syndrome (ARDS), asthma, asthma bronchitis, bronchiectasis, diffuse interstitial lung disorders, pneumoconioses, fibrosing aveolitis, lung fibrosis,
disorders associated with skin and connective tissue conditions
e.g. including eczema, atopic dermatitis, contact dermatitis, psoriasis, acne, dermatomyositis, Sjörgen's syndrome, Churg-Strauss syndrome, sunburn, skin cancer, wound healing, urticaria, toxic epidermal necrolysis,
disorders associated with allergic conditions,
e.g. including delayed-type hypersensitivity, allergic conjunctivitis, drug allergies, rhinitis, allergic rhinitis, vasculitis, contact dermatitis;
disorders associated with angiogenesis,
e.g. including insufficient ability to recruit blood supply, disorders characterized by modified angiogenesis, tumor associated angiogenesis,
disorders associated with cancer and cell overproliferation,
e.g. including premalignant conditions, hyperproliferative disorders, all type of cancers, cancers whether primary or metastatic, cervical and metastatic cancer, cancer originating from uncontrolled cellular proliferation, solid tumors, unresponsiveness to normal death-inducing signals (immortalization), increased cellular motility and invasiveness, increased ability to recruit blood supply through induction of new blood vessel formation (angiogenesis), genetic instability, dysregulated gene expression, solid tumors, such as described in WO02066019, including non-small cell lung cancer, cervical cancer; tumor growth, lymphoma, B-cell or T-cell lymphoma, benign tumors, benign dysproliferative disorders, renal carcinoma, esophageal cancer, stomach cancer, renal carcinoma, bladder cancer, breast cancer, colon cancer, lung cancer, melanoma, nasopharyngeal cancer, osteocarcinoma, ovarian cancer, uterine cancer; prostate cancer, skin cancer, leukemia, tumor neovascularization, angiomas, myelodysplastic disorders, unresponsiveness to normal death-inducing signals (immortalization), increased cellular motility and invasiveness, genetic instability, dysregulated gene expression, (neuro)endocrine cancer (carcinoids), blood cancer, lymphocytic leukemias, neuroblastoma; soft tissue cancer, cancer prevention, e.g. prevention of metastasis,
disorders associated with infectious disorders, e.g. with chronic infectious conditions,
e.g. including bacterial disorders, otitis media, Lyme disease, thryoditis, viral disorders, parasitic disorders, fungal disorders, malaria, e.g. malaria anemia, sepsis, severe sepsis, septic shock, e.g. endotoxin-induced septic shock, exotoxin-induced toxic shock, infective (true septic) shock, septic shock caused by Gram-negative bacteria, pelvic inflammatory disease, AIDS, enteritis, pneumonia; meningitis, encephalitis,
disorders associated with myasthenia gravis,
disorders associated with nephritis,
e.g. including glomerulonephritis, interstitial nephritis, Wegener's granulomatosis, fibrosis,
disorders associated with diabetic conditions,
e.g. including diabetes (type I diabetes, type II diabetes, gestational diabetes), diabetic retinopathy, insulin-dependent diabetes, diabetes mellitus, gestational diabetes), insulin hyposecretion, obesity;
disorders associated with endiometriosis, testicular dysfunctions,
disorders associated with pain,
e.g. associated with CNS disorders, such as multiple sclerosis, spinal cord injury, sciatica, failed back surgery syndrome, traumatic brain injury, epilepsy, Parkinson's disease, post-stroke, and vascular lesions in the brain and spinal cord (e.g., infarct, hemorrhage, vascular malformation);
non-central neuropathic pain, e.g. including that associated with post mastectomy pain, phantom feeling, reflex sympathetic dystrophy (RSD), trigeminal neuralgiaradioculopathy, post-surgical pain, HIV/AIDS related pain, cancer pain, metabolic neuropathies (e.g., diabetic neuropathy, vasculitic neuropathy secondary to connective tissue disease), paraneoplastic polyneuropathy associated, for example, with carcinoma of lung, or leukemia, or lymphoma, or carcinoma of prostate, colon or stomach, trigeminal neuralgia, cranial neuralgias, and post-herpetic neuralgia;
pain associated with peripheral nerve damage, central pain (i.e. due to cerebral ischemia) and various chronic pain i.e. lumbago, back pain (low back pain), inflammatory and/or rheumatic pain;
headache pain (for example, migraine with aura, migraine without aura, and other migraine disorders), episodic and chronic tension-type headache, tension-type like headache, cluster headache, and chronic paroxysmal hemicrania;
visceral pain such as pancreatits, intestinal cystitis, dysmenorrhea, irritable Bowel syndrome, Crohn's disease, biliary colic, ureteral colic, myocardial infarction and pain syndromes of the pelvic cavity, e.g., vulvodynia, orchialgia, urethral syndrome 15 and protatodynia;
acute pain, for example postoperative pain, and pain after trauma;
disorders associated with rheumatic disorders,
e.g. including arthritis, rheumatoid arthritis, osteoarthritis, psoriatic arthritis, crystal arthropathies, gout, pseudogout, calcium pyrophosphate deposition disease, lupus syndromes, systemic lupus erythematosus, sclerosis, sclerodema, multiple sclerosis, artherosclerosis, arteriosclerosis, spondyloarthropathies, systemic sclerosis, reactive arthritis, Reiter's syndrome, ankylosing spondylitis, polymyositis,
disorders associated with sarcoidosis,
disorders associated with transplantation,
e.g. including transplant rejection crisis and other disorders following transplantation, such as organ or tissue (xeno)transplant rejection, e.g. for the treatment of recipients of e.g. heart, lung, combined heart-lung, liver, kidney, pancreatic, skin, corneal transplants, graft versus host disease, such as following bone marrow transplantation, ischemic reperfusion injury,

Disorders mediated by, e.g. insufficient, GPBAR1 activity which are prone to be successfully treated with GPBAR1 agonists, such as specific GBPAR1 activating compounds of the present invention, preferably include inflammatory, immune, e.g. autoimmune and allergic disorders, such as rheumatoid arthritis, inflammatory bowel disease, systemic lupus erytomatosis, multiple sclerosis, transplant rejection crisis, psoriasis, cancer, AIDS, diabetes (diabetes type II), obesity; more preferably rheumatoid arthritis, systemic lupus erytomatosis, multiple sclerosis, psoriasis, diabetes (diabetes type II), obesity; e.g. psoriasis.

In another aspect the present invention provides
a compound of the present invention for use as a pharmaceutical,
the use of a compound of the present invention as a pharmaceutical;
e.g. for the treatment of disorders mediated by, e.g. insufficient, GPBAR1 activity, wherein for the treatment of disorders mediated by, e.g. insufficient, GPBAR1 activity a compound of the present invention is a specific GBPAR1 activating compound of the present invention.

In another aspect the present invention provides the compounds

N-(2-Ethynyl-benzyl)-3-methyl-N-phenyl-isonicotinamide, and

N-(4-Ethynyl-benzyl)-3-methyl-N-phenyl-isonicotinamide,

e.g. in free form or in the form of a salt, e.g. wherein salts preferably include pharmaceutically acceptable salts, although other salts, e.g. for production/purifaction/isolation processes, are included, e.g. and wherein solvates, e.g. of a free form or of a salt form are included.

For pharmaceutical use one or more compounds of the present invention may be used, e.g. one, or a combination of two or more compounds of the present invention, or specific GPBAR1-compound(s) of the present invention, preferably one compound of the present invention or specific GPBAR1-compound of the present invention is used.

A compound of the present invention or a specific GBPAR1 activating compound of the present invention may be used as a pharmaceutical in the form of a pharmaceutical composition.

In another aspect the present invention provides a pharmaceutical composition comprising a compound of the present invention in association with at least one pharmaceutically acceptable excipient, e.g. appropriate carrier and/or diluent, e.g. including fillers, binders, disintegrators, flow conditioners, lubricants, sugars or sweeteners, fragrances, preservatives, stabilizers, wetting agents and/or emulsifiers, solubilizers, salts for regulating osmotic pressure and/or buffers.

In another aspect the present invention provides a pharmaceutical composition comprising the compound N-(2-ethynyl-benzyl)-3-methyl-N-phenyl-isonicotinamide or N-(4-ethynyl-benzyl)-3-methyl-N-phenyl-isonicotinamide, e.g. in free form or in the form of a salt, in association with at least one pharmaceutically acceptable excipient, e.g. appropriate carrier and/or diluent, e.g. including fillers, binders, disintegrators, flow conditioners, lubricants, sugars or sweeteners, fragrances, preservatives, stabilizers, wetting agents and/or emulsifiers, solubilizers, salts for regulating osmotic pressure and/or buffers.

A pharmaceutical composition provided by the present invention is herein also designated as “pharmaceutical composition of (according to) the present invention”.

In another aspect the present invention provides
a pharmaceutical composition of the present invention for use of treating disorders which are mediated by, e.g. insufficient, GPBAR1 activity;
the use of a pharmaceutical composition of the present invention for treating disorders which are mediated by, e.g. insufficient, GPBAR1 activity,
   e.g. wherein the pharmaceutical composition comprises a specific GBPAR1 activating compound of the present invention, namely a compound of formula I wherein R₃ is as defined above and additionally R₃ denotes (C₂₋₄)alkynyl-phenyl.

In a further aspect the present invention provides a method of treating disorders which are mediated by, e.g. insufficient, GPBAR1 activity, e.g. including disorders as specified above, which treatment comprises administering to a subject in need of such treatment a therapeutically effective amount of a specific GBPAR1 activating compound of the present invention; e.g. in the form of a pharmaceutical composition.

In another aspect the present invention provides
a specific GBPAR1 activating compound of the present invention for the manufacture of a medicament,
the use of a specific GBPAR1 activating compound of the present invention for the manufacture of a medicament,
   e.g. wherein the medicament comprises a pharmaceutical composition according to the present invention,
   for the treatment of disorders, which are mediated by, e.g. insufficient, GPBAR1 activity.

Treatment includes treatment and prophylaxis (prevention).

For such treatment, the appropriate dosage will, of course, vary depending upon, for example, the chemical nature and the pharmacokinetic data of a compound of the present invention used, the individual host, the mode of administration and the nature and severity of the conditions being treated. However, in general, for satisfactory results in larger mammals, for example humans, an indicated daily dosage includes a range
from about 0.001 g to about 1.5 g, such as 0.001 g to 1.5 g;
from about 0.01 mg/kg body weight to about 20 mg/kg body weight, such as 0.01 mg/kg body weight to 20 mg/kg body weight,
   for example administered in divided doses up to four times a day.

A compound of the present invention may be administered to larger mammals, for example humans, by similar modes of administration, e.g. at similar dosages, than conventionally used or indicated for other mediators, e.g. low molecular weight activators, of GPBAR1 activity.

A compound of the present invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration;

e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

The compounds of the present invention may be administered in the form of a pharmaceutically acceptable salt, or in free form; optionally in the form of a solvate. A compound of the present invention in the form of a salt and/or in the form of a solvate exhibit the same order of activity as a compound of the present invention in free form.

A compound of the present invention may be administered by any conventional route, for example enterally, e.g. including nasal, buccal, rectal, oral administration; parenterally, e.g. including intravenous, intraarterial, intramuscular, intracardiac, subcutanous, intraosseous infusion, transdermal (diffusion through the intact skin), transmucosal (diffusion through a mucous membrane), inhalational administration; topically; e.g. including epicutaneous, intranasal, intratracheal administration; intraperitoneal (infusion or injection into the peritoneal cavity); epidural (peridural) (injection or infusion into the epidural space); intrathecal (injection or infusion into the cerebrospinal fluid); intravitreal (administration via the eye); or via medical devices, e.g. for local delivery, e.g. stents.

For topical use, e.g. including administration to the eye, satisfactory results may be obtained with local administration of a 0.5-10%, such as 1-3% concentration of active substance several times daily, e.g. 2 to 5 times daily.

A compound of the present invention may be used for any method or use as described herein alone or in combination with one or more, at least one, other, second drug substance.

In another aspect the present invention provides
A combination of a compound of the present invention with at least one second drug substance;
A pharmaceutical combination comprising a compound of the present invention in combination with at least one second drug substance;
A pharmaceutical composition comprising a compound of the present invention in combination with at least one second drug substance and one or more pharmaceutically acceptable excipient(s);
A compound of the present invention in combination with at least one second drug substance, e.g. in the form of a pharmaceutical combination or composition, for use in any method as defined herein, e.g.
   A combination, a pharmaceutical combination or a pharmaceutical composition, comprising a compound of the present invention and at least one second drug substance for use as a pharmaceutical;
   The use as a pharmaceutical of a compound of the present invention in combination with at least one second drug substance, e.g. in the form of a pharmaceutical combination or composition;
   The use of a compound of the present invention for the manufacture of a medicament for use in combination with a second drug substance,
A method for treating disorders mediated by, e.g. insufficient, GPBAR1 activity in a subject in need thereof, comprising co-administering, concomitantly or in sequence, a therapeutically effective amount of a specific GBPAR1 activating compound of the present invention and at least one second drug substance, e.g. in the form of a pharmaceutical combination or composition;
A specific GBPAR1 activating compound of the present invention in combination with at least one second drug substance, e.g. in the form of a pharmaceutical combination or composition, for use in the preparation of a medicament for use in disorders mediated by, e.g. insufficient, GPBAR1 activity.

Combinations include fixed combinations, in which a compound of the present invention or a specific GBPAR1 activating compound of the present invention and at least one second drug substance are in the same formulation; kits, in which a compound of the present invention or a specific GBPAR1 activating compound of the present invention and at least one second drug substance in separate formulations are provided in the same package, e.g. with instruction for co-administration; and free combinations in which a compound of the present or a specific GBPAR1 activating compound of the present invention and at least one second drug substance are packaged separately, but instruction for concomitant or sequential administration are given.

In another aspect the present invention provides
A pharmaceutical package comprising a first drug substance which is a compound of the present invention and at least one second drug substance, beside instructions for combined administration;
A pharmaceutical package comprising a compound of the present invention beside instructions for combined administration with at least one second drug substance;
A pharmaceutical package comprising at least one second drug substance beside instructions for combined administration with a compound of the present invention;
A pharmaceutical package comprising a first drug substance which is a specific GBPAR1 activating compound of the present invention and at least one second drug substance, beside instructions for combined administration, for use in the treatment of disorders mediated by, e.g. insufficient, GPBAR1 activity;
A pharmaceutical package comprising a specific GBPAR1 activating compound of the present invention beside instructions for combined administration with at least one second drug substance, for use in the treatment of disorders mediated by, e.g. insufficient, GPBAR1 activity;
A pharmaceutical package comprising at least one second drug substance beside instructions for combined administration with a specific GBPAR1 activating compound of the present invention, for use in the treatment of disorders mediated by, e.g. insufficient, GPBAR1 activity.

Treatment with combinations according to the present invention may provide improvements compared with single treatment.

In another aspect the present invention provides
A pharmaceutical combination comprising an amount of a compound of the present invention and an amount of a second drug substance, wherein the amounts are appropriate to produce a synergistic therapeutic effect;
A method for improving the therapeutic utility of a compound of the present invention comprising co-administering, e.g. concomitantly or in sequence, of a therapeutically effective amount of a compound of the present invention and a second drug substance.
A method for improving the therapeutic utility of a second drug substance comprising co-administering, e.g. concomitantly or in sequence, of a therapeutically effective amount of a compound of the present invention and a second drug substance;
A pharmaceutical combination comprising an amount of a specific GBPAR1 activating compound of the present invention and an amount of a second drug substance, wherein the amounts are appropriate to produce a synergistic therapeutic effect, for use in the treatment of disorders mediated by, e.g. insufficient, GPBAR1 activity.
A method for improving the therapeutic utility of a specific GBPAR1 activating compound of the present invention comprising co-administering, e.g. concomitantly or in sequence, of a therapeutically effective amount of a compound of the present invention and a second drug substance, for use in the treatment of disorders mediated by, e.g. insufficient, GPBAR1 activity.
A method for improving the therapeutic utility of a second drug substance comprising co-administering, e.g. concomitantly or in sequence, of a therapeutically effective amount of a specific GBPAR1 activating compound of the present invention and a second drug substance, for use in the treatment of disorders mediated by, e.g. insufficient, GPBAR1 activity.

A combination of the present invention and a second drug substance as a combination partner may be administered by any conventional route, for example as set out above for a compound of the present invention. A second drug may be administered in dosages as appropriate, e.g. in dosage ranges which are similar to those used for single treatment, or, e.g. in case of synergy, even below conventional dosage ranges.

Pharmaceutical compositions according to the present invention may be manufactured according, e.g. analogously, to a method as conventional, e.g. by mixing, granulating, coating, dissolving or lyophilizing processes. Unit dosage forms may contain, for example, from about 0.1 mg to about 1500 mg, such as 1 mg to about 1000 mg.

Pharmaceutical compositions comprising a combination of the present invention and pharmaceutical compositions comprising a second drug as described herein, may be provided as appropriate, e.g. according, e.g. analogously, to a method as conventional, or as described herein for a pharmaceutical composition of the present invention.

By the term “second drug substance” is meant a chemotherapeutic drug, especially any chemotherapeutic agent other than a specific GBPAR1 activating compound of the present invention.

For example, a second drug substance as used herein includes anti-inflammatory and/or immunomodulatory and/or anticancer drugs, e.g. including antiviral drugs, e.g. and/or anesthetics.

Anti-inflammatory and/or immunomodulatory drugs which are prone to be useful in combination with a specific GBPAR1 activating compound of the present invention e.g include
mediators, e.g. inhibitors, of mTOR activity, including rapamycin of formula

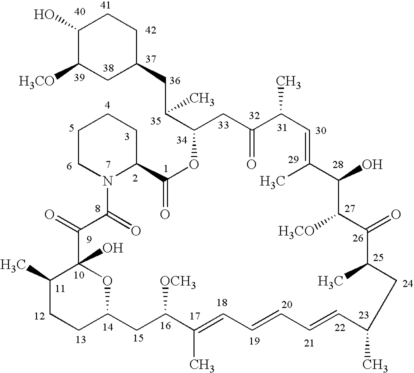

and rapamycin derivatives, e.g. including
40-O-alkyl-rapamycin derivatives, such as 40-O-hydroxyalkyl-rapamycin derivatives, such as 40-O-(2-hydroxy)-ethyl-rapamycin (everolimus),
32-deoxo-rapamycin derivatives and 32-hydroxy-rapamycin derivatives, such as 32-deoxorapamycin,
16-O-substituted rapamycin derivatives such as 16-pent-2-ynyloxy-32-deoxorapamycin, 16-pent-2-ynyloxy-32 (S or R)-dihydro-rapamycin, 16-pent-2-ynyloxy-32(S or R)-dihydro-40-O-(2-hydroxyethyl)-rapamycin,
rapamycin derivatives which are acylated at the oxygen group in position 40, e.g. 40-[3-hydroxy-2-(hydroxy-methyl)-2-methylpropanoate]-rapamycin (also known as CCI779), rapamycin derivatives which are substituted in 40 position by heterocyclyl, e.g. 40-epi-(tetrazolyl)-rapamycin (also known as ABT578),
the so-called rapalogs, e.g. as disclosed in WO9802441, WO0114387 and WO0364383, such as AP23573, and
compounds disclosed under the name TAFA-93, AP23464, AP23675, AP23841 and biolimus (e.g. biolimus A9).
mediators, e.g. inhibitors, of calcineurin, e.g. cyclosporin A, FK 506;
ascomycins having immuno-suppressive properties, e.g. ABT-281, ASM981;
corticosteroids; cyclophosphamide; azathioprene; leflunomide; mizoribine;
mycophenolic acid or salt; e.g. sodium, mycophenolate mofetil;
15-deoxyspergualine or an immunosuppressive homologue, analogue or derivative thereof;
mediators, e.g. inhibitors, of bcr-abl tyrosine kinase activity;
mediators, e.g. inhibitors, of c-kit receptor tyrosine kinase activity;
mediators, e.g. inhibitors, of PDGF receptor tyrosine kinase activity, e.g. Gleevec (imatinib);
mediators, e.g. inhibitors, of p38 MAP kinase activity,
mediators, e.g. inhibitors, of VEGF receptor tyrosine kinase activity,
mediators, e.g. inhibitors, of PKC activity, e.g. as disclosed in WO0238561 or WO0382859, e.g. the compound of Example 56 or 70;
mediators, e.g. inhibitors, of JAK3 kinase activity, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO2004052359 or WO2005066156;
mediators, e.g. agonists or modulators of S1P receptor activity, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts;
immunosuppressive monoclonal antibodies, e.g., monoclonal antibodies to leukocyte receptors, e.g., Blys/BAFF receptor, MHC, CD2, CD3, e.g. visilizumab, CD4, e.g. zanolimumab, CD7, CD8, CD11a, e.g. efalizumab (Raptiva®), CD20, e.g. rituximab (Rituxan®, ibritumomab tiuxetan conjugated to ¹¹¹In or ⁹⁰Y (Zevalin®), ¹³¹I tositumumab (Bexxar®), CD25, CD28, CD33, e.g. gemtuzumab (Mylotarg®, CD40, CD45, CD52, CD54, e.g. Alemtuzumab (Campath-I®), CD58, CD80, CD86, IL-2 receptor, e.g. dacluzimab, IL6 receptor (e.g. tocilizumab), IL-12 receptor, IL-17 receptor, IL-23 receptor or their ligands;
other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; or an anti-CTLA4 agent, such as ipilimumab, ticilimumab,
mediators, e.g. inhibitors of adhesion molecule activities, e.g. LFA-1 antagonists, ICAM-1 or -3 antagonists, VCAM-4 antagonists or VLA-4 antagonists,
mediators, e.g. antagonists of CCR9 acitiviy,
mediators, e.g. inhibitors, of MIF activity,
5-aminosalicylate (5-ASA) agents, such as sulfasalazine, Azulfidine®, Asacol®, Dipentum®, Pentasa®, Rowasa®, Canasa®, Colazal®, e.g. drugs containing mesalamine; e.g mesalazine in combination with heparin;
mediators, e.g. inhibitors, of TNF-alpha activity, e.g. including antibodies which bind to TNF-alpha, e.g. infliximab (Remicadee), thalidomide, lenalidomide, golimumab, adalimumab (Humira®, fully human immunoglobulin G (IgG1) monoclonal antibody that is specific for human TNF alpha), etanercept (Enbrel®), alefacept (Amevive®), certolizumab pegol (Cimzia®, CDP 870), afelimomab, AME527 (Lilly),
nitric oxide releasing non-steriodal anti-inflammatory drugs (NSAIDs), e.g. including COX-inhibiting NO-donating drugs (CINOD);
phosphordiesterase, e.g. mediators, such as inhibitors of PDE4B activity,
mediators, e.g. inhibitors, of caspase activity,
mediators, e.g. agonists, of the G protein coupled receptor GPBAR1,
mediators, e.g. inhibitors, of ceramide kinase activity,
‘multi-functional anti-inflammatory’ drugs (MFAIDs), e.g. cytosolic phospholipase A2 (cPLA2) inhibitors, such as membrane-anchored phospholipase A2 inhibitors linked to glycosaminoglycans;
antibiotics, such as penicillins, cephalosporins, erythromycins, tetracyclines, sulfonamides, such as sulfadiazine, sulfisoxazole; sulfones, such as dapsone; pleuromutilins, fluoroquinolones, e.g. metronidazole, quinolones such as ciprofloxacin; levofloxacin; probiotics and commensal bacteria e.g. Lactobacillus, Lactobacillus reuteri;
antiviral drugs, such as ribivirin, vidarabine, acyclovir, ganciclovir, zanamivir, oseltamivir phosphate, famciclovir, atazanavir, amantadine, didanosine, efavirenz, foscarnet, indinavir, lamivudine, nelfinavir, ritonavir, saquinavir, stavudine, valacyclovir, valganciclovir, civacir, zidovudine, antibodies against RSV protein, e.g. RSV F protein, such as palivizumab (Synagis®), motavizumab,
mediators, e.g. inhibitors of the blood protein “complement 5(a)”, such as eculizumab,
pexelizumab,
serum phosphorus controlling agents, e.g. sevelamer carbonate (Renagel®),; phosphate binders that reduces high serum phosphate levels in renal disease patients, such as lanthanum carbonate (Fosrenol®).
mediators, e.g. agonists, of GPBAR1 mediator activity, e.g. including antibodies and low molecular weight compounds which are different from a specific GBPAR1 activating compound of the present invention,
mediators, e.g. inhibitors of ceramide kinase activity, e.g. including antibodies and low molecular weight compounds,
alpha-4-integrin antibodies, e.g. natalizumab (Tysabri®.

Anti-inflammatory drugs which are prone to be useful in combination with a specific GBPAR1 activating compound of the present invention include e.g. non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); cyclooxygenase-2 (COX-2) inhibitors such as celecoxib; inhibitors of phosphodiesterase type IV (PDE-IV); antagonists of the chemokine receptors, especially CCR1, CCR2, and CCR3; cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrate, fenofibrate and benzafibrate), and probucol; anticholinergic agents such as muscarinic antagonists (ipratropium bromide); other compounds such as theophylline, sulfasalazine and aminosalicylates, e.g. 5-aminosalicylic acid and prodrugs thereof, antirheumatics, IgE antibodies, e.g. omalizumab (Xolair®.

Antiallergic drugs which are prone to be useful in combination with a specific GBPAR1 activating compound of the present invention include e.g. antihistamines (H1-histamine antagonists), e.g. bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); bronchodilators, antiasthmatics (mast cell stabilizers).

Anesthetics which are prone to be useful as a combination partner with a specific GBPAR1 activating compound of the present invention e.g. include ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine.

Anticancer drugs which are prone to be useful as a combination partner with which are prone to be useful in combination with a specific GBPAR1 activating compound of the present invention, e.g. prone to be useful according to the present invention, e.g. include
i. a steroid; e.g. prednisone.
ii. an adenosine-kinase-inhibitor; which targets, decreases or inhibits nucleobase, nucleoside, nucleotide and nucleic acid metabolisms, such as 5-Iodotubercidin, which is also known as 7H-pyrrolo[2,3-d]pyrimidin-4-amine, 5-iodo-7-β-D-ribofuranosyl.
iii. an adjuvant; which enhances the 5-FU-TS bond as well as a compound which targets, decreases or inhibits, alkaline phosphatase, such as leucovorin, levamisole.
iv. an adrenal cortex antagonist; which targets, decreases or inhibits the activity of the adrenal cortex and changes the peripheral metabolism of corticosteroids, resulting in a decrease in 17-hydroxycorticosteroids, such as mitotane.
v. an AKT pathway inhibitor; such as a compound which targets, decreases or inhibits Akt, also known as protein kinase B (PKB), such as deguelin, which is also known as 3H-bis[1]benzopyrano[3,4-b:6′,5′-e]pyran-7(7aH)-one, 13,13a-dihydro-9,10-dimethoxy-3,3-dimethyl-, (7aS,13aS); and triciribine, which is also known as 1,4,5,6,8-pentaazaacenaphthylen-3-amine, 1,5-dihydro-5-methyl-1-β-D-ribofuranosyl; KP372-1 (QLT394).
vi. an alkylating agent; which causes alkylation of DNA and results in breaks in the DNA molecules as well as cross-linking of the twin strands, thus interfering with DNA replication and transcription of RNA, such as chlorambucil, chlormethine, cyclophosphamide, ifosfamide, melphalan, estramustine; nitrosueras, such as carmustine, fotemustine, lomustine, streptozocin (streptozotocin, STZ), BCNU; Gliadel; dacarbazine, mechlorethamine, e.g. in the form of a hydrochloride, procarbazine, e.g. in the form of a hydrochloride, thiotepa, temozolomide, nitrogen mustard, mitomycin, altretamine, busulfan, estramustine, uramustine. Cyclophosphamide can be administered, e.g., in the form as it is marketed, e.g., under the trademark CYCLOSTIN®; ifosfamide as HOLOXAN®, temozolomide as TEMODAR®, nitrogen mustard as MUSTARGEN®, estramustine as EMYCT®, streptozocin as ZANOSAR®.
vii. an angiogenesis inhibitor; which targets, decreases or inhibits the production of new blood vessels, e.g. which targets methionine aminopeptidase-2 (MetAP-2), macrophage inflammatory protein-1 (MIP-1alpha), CCL5, TGF-beta, lipoxygenase, cyclooxygenase, and topoisomerase, or which indirectly targets p21, p53, CDK2 and collagen synthesis, e.g. including fumagillin, which is known as 2,4,6,8-decatetraenedioic acid, mono[(3R,4S,5S,6R)-5-methoxy-4-[(2R,3R)-2-methyl-3-(3-methyl-2-butenyl)oxiranyl]-1-oxaspiro[2.5]oct-6-yl]ester, (2E,4E,6E,8E)-(9Cl); shikonin, which is also known as 1,4-naphthalenedione, 5,8-dihydroxy-2-[(1R)-1-hydroxy-4-methyl-3-pentenyl]-(9Cl); tranilast, which is also known as benzoic acid, 2-[[3-(3,4-dimethoxyphenyl)-1-oxo-2-propenyl]amino]; ursolic acid; suramin; bengamide or a derivative thereof, thalidomide, TNP-470.
viii. an anti-androgen; which blocks the action of androgens of adrenal and testicular origin which stimulate the growth of normal and malignant prostatic tissue, such as nilutamide; bicalutamide (CASODEX®), which can be formulated, e.g., as disclosed in U.S. Pat. No. 4,636,505.
ix. an anti-estrogen; which antagonizes the effect of estrogens at the estrogen receptor level, e.g. including an aromatase inhibitor, which inhibits the estrogen production, i.e. the conversion of the substrates androstenedione and testosterone to estrone and estradiol, respectively,
   e.g. including atamestane, exemestane, formestane, aminoglutethimide, roglethimide, pyridoglutethimide, trilostane, testolactone, ketokonazole, vorozole, fadrozole, anastrozole, letrozole, toremifene; bicalutamide; flutamide; tamoxifen, tamoxifen citrate; tamoxifen; fulvestrant; raloxifene, raloxifene hydrochloride. Tamoxifen may be e.g. administered in the form as it is marketed, e.g., NOLVADEX®; and raloxifene hydrochloride is marketed as EVISTA®. Fulvestrant may be formulated as disclosed in U.S. Pat. No. 4,659,516 and is marketed as FASLODEX®.
x. an anti-hypercalcemia agent; which is used to treat hypercalcemia, such as gallium (III) nitrate hydrate; and pamidronate disodium.
xi. an antimetabolite; which inhibits or disrupts the synthesis of DNA resulting in cell death, such as folic acids, e.g. methotrexate, pemetrexed, raltitrexed; purins, e.g. 6-mercaptopurine, cladribine, clofarabine; fludarabine, thioguanine (tioguanine), 6-thioguanine, nelarabine (compound 506), tiazofurin (inhibits inosine monophosphate dehydrogenase and guanosine triphosphate pools), pentostatin (deoxycoformycin); cytarabine; flexuridine; fluorouracil; 5-fluorouracil (5-FU), floxuridine (5-FUdR), capecitabine; gemcitabine; gemcitabine hydrochloride; hydroxyurea (e.g. Hydrea®); DNA de-methylating agents, such as 5-azacytidine (Vidaza®) and decitabine; fluoromethylene deoxycitidine (FmdC), 5-aza-2′-deoxycytidine, troxacitabine (L-isomer cytosine analogue), edatrexate; Capecitabine and gemcitabine can be administered e.g. in the marketed form, such as XELODA® and GEMZAR®.
xii. an apoptosis inducer; which induces the normal series of events in a cell that leads to its death, e.g. selectively inducing the X-linked mammalian inhibitor of apoptosis protein XIAP, or e.g. downregulating BCL-xL; such as ethanol, 2-[[3-(2,3-dichlorophenoxy)propyl]amino]; gambogic acid; embelin, which is also known as 2,5-cyclohexadiene-1,4-dione, 2,5-dihydroxy-3-undecyl-(9Cl); arsenic trioxide arsenic trioxide (TRISENOX®).
xiii. an aurora kinase inhibitor; which targets, decreases or inhibits later stages of the cell cycle from the G2/M check point all the way through to the mitotic checkpoint and late mitosis; such as binucleine 2, which is also known as methanimidamide, N′-[1-(3-chloro-4-fluorophenyl)-4-cyano-1H-pyrazol-5-yl]-N,N-dimethyl-(9Cl).
xiv. a Bruton's Tyrosine Kinase (BTK) inhibitor; which targets, decreases or inhibits human and murine B cell development; such as terreic acid.
xv. a calcineurin inhibitor; which targets, decreases or inhibits the T cell activation pathway, such as cypermethrin, which is also known as cyclopropanecarboxylic acid, 3-(2,2-dichloroethenyl)-2,2-dimethyl-, cyano(3-phenoxyphenyl)methyl ester; deltamethrin, which is also known as cyclopropanecarboxylic ad, 3-(2,2-dibromoethenyl)-2,2-dimethyl-(S)-cyano(3-phenoxyphenyl)methyl ester, (1R,3R); fenvalerate, which is also known as benzeneacetic acid, 4-chloro-α-(1-methylethyl)-, cyano(3-phenoxyphenyl)methyl ester; and Tyrphostin 8; but excluding cyclosporin or FK506.
xvi. a CaM kinase II inhibitor; which targets, decreases or inhibits CaM kinases; constituting a family of structurally related enzymes that include phosphorylase kinase, myosin light chain kinase, and CaM kinases I-IV; such as 5-isoquinolinesulfonic acid, 4-[(2S)-2-[(5-isoquinolinylsulfonyl)methylamino]-3-oxo-3-(4-phenyl-1-piperazinyl)propyl]phenyl ester (9Cl); benzenesulfonamide, N-[2-[([3-(4-chlorophenyl)-2-propenyl]methyl]amino]methyl]phenyl]-N-(2-hydroxyethyl)-4-methoxy.
xvii. a CD45 tyrosine phosphatase inhibitor; which targets, decreases or inhibits dephosphorylating regulatory pTyr residues on Src-family protein-tyrosine kinases, which aids in the treatment of a variety of inflammatory and immune disorders; such as phosphonic acid, [[2-(4-bromophenoxy)-5-nitrophenyl]hydroxymethyl].
xviii. a CDC25 phosphatase inhibitor; which targets, decreases or inhibits overexpressed dephosphorylate cyclin-dependent kinases in tumors; such as 1,4-naphthalenedione, 2,3-bis[(2-hydroyethyl)thio].
xix. a CHK kinase inhibitor; which targets, decreases or inhibits overexpression of the antiapoptotic protein Bcl-2; such as debromohymenialdisine. Targets of a CHK kinase inhibitor are CHK1 and/or CHK2.
xx. a controlling agent for regulating genistein, olomucine and/or tyrphostins; such as daidzein, which is also known as 4H-1-benzopyran-4-one, 7-hydroxy-3-(4-hydroxyphenyl); Iso-Olomoucine, and Tyrphostin 1.
xxi. a cyclooxygenase inhibitor; e.g. including Cox-2 inhibitors; which targets, decreases or inhibits the enzyme Cox-2 (cyclooxygenase-2); such as 1H-indole-3-acetamide, 144-chlorobenzoyl)-5-methoxy-2-methyl-N-(2-phenylethyl); 5-alkyl substituted 2-arylaminophenylacetic acid and derivatives, e.g. celecoxib (CELEBREX™), rofecoxib (VIOXX®), etoricoxib, valdecoxib; or a 5-alkyl-2-arylaminophenylacetic acid, e.g., 5-methyl-2-(2′-chloro-6′-fluoroanilino)phenyl acetic acid, lumiracoxib; and celecoxib.
xxii. a cRAF kinase inhibitor, which targets, decreases or inhibits the up-regulation of E-selectin and vascular adhesion molecule-1 induced by TNF; such as 3-(3,5-dibromo-4-hydroxybenzylidene)-5-iodo-1,3-dihydroindol-2-one; and benzamide, 3-(dimethylamino)-N-[3-[(4-hydroxybenzoyl)amino]-4-methylphenyl]. Raf kinases play an important role as extracellular signal-regulating kinases in cell differentiation, proliferation, and apoptosis. A target of a cRAF kinase inhibitor includes, but is not limited, to RAF1.
xxiii. a cyclin dependent kinase inhibitor, which targets, decreases or inhibits cyclin dependent kinase playing a role in the regulation of the mammalian cell cycle; such as N9-isopropyl-olomoucine; olomoucine; purvalanol B, which is also known as Benzoic acid, 2-chloro-4-[[2-[[(1R)-1-(hydroxymethyl)-2-methylpropyl]amino]-9-(1-methylethyl)-9H-purin-6-yl]amino]-(9Cl); roascovitine; indirubin, which is also known as 2H-indol-2-one, 3-(1,3-dihydro-3-oxo-2H-indol-2-ylidene)-1,3-dihydro-(9Cl); kenpaullone, which is also known as indolo[3,2-d][1]benzazepin-6(5H)-one, 9-bromo-7,12-dihydro-(9Cl); purvalanol A, which is also known as 1-Butanol, 2-[[6-[(3-chlorophenyl)amino]-9-(1-methylethyl)-9H-purin-2-yl]amino]-3-methyl-, (2R)-(9Cl); indirubin-3′-monooxime. Cell cycle progression is regulated by a series of sequential events that include the activation and subsequent inactivation of cyclin dependent kinases (Cdks) and cyclins. Cdks are a group of serine/threonine kinases that form active heterodimeric complexes by binding to their regulatory subunits, cyclins. Examples of targets of a cyclin dependent kinase inhibitor include, but are not limited to, CDK, AHR, CDK1, CDK2, CDK5, CDK4/6, GSK3beta, and ERK.
xxiv. a cysteine protease inhibitor; which targets, decreases or inhibits cystein protease which plays a vital role in mammalian cellular turnover and apotosis; such as 4-morpholinecarboxamide, N-[(1S)-3-fluoro-2-oxo-1-(2-phenylethyl)propyl]amino]-2-oxo-1-(phenylmethy)ethyl].
xxv. a DNA intercalator; which binds to DNA and inhibits DNA, RNA, and protein synthesis; such as plicamycin, dactinomycin.
xxvi. a DNA strand breaker; which causes DNA strand scission and results in inhibition of DNA synthesis, inhibition of RNA and protein synthesis; such as bleomycin.
xxvii. an E3 Ligase inhibitor; which targets, decreases or inhibits the E3 ligase which inhibits the transfer of ubiquitin chains to proteins, marking them for degradation in the proteasome; such as N-((3,3,3-trifluoro-2-trifluoromethyl)propionyl)sulfanilamide.
xxviii. an endocrine hormone; which by acting mainly on the pituitary gland causes the suppression of hormones in males, the net effect being a reduction of testosterone to castration levels; in females, both ovarian estrogen and androgen synthesis being inhibited; such as leuprolide; megestrol, megestrol acetate.
xxix. compounds targeting, decreasing or inhibiting the activity of the epidermal growth factor family of receptor tyrosine kinases (EGFR, ErbB2, (HER-2), ErbB3, ErbB4 as homo- or heterodimers), such as compounds, proteins or antibodies which inhibit members of the EGF receptor tyrosine kinase family, e.g. EGF receptor, ErbB1, ErbB2, ErbB3 and ErbB4 or bind to EGF or EGF-related ligands, and are in particular those compounds, proteins or monoclonal antibodies generically and specifically disclosed in WO 9702266, e.g. the compound of ex. 39, EP0564409, WO9903854, EP0520722, EP0566226, EP0787722, EP0837063, U.S. Pat. No. 5,747,498, WO9810767, WO9730034, WO9749688, WO9738983 and, especially, WO9630347, e.g. a compound known as CP 358774, WO9633980, e.g. a compound known as ZD 1839; and WO 9503283, e.g. a compound known as ZM105180, e.g including the dual acting tyrosine kinase inhibitor (ErbB1 and ErbB2) lapatinib (GSK572016), e.g. lapatinib ditosylate; panituzumab, trastuzumab (HERCEPTIN®), cetuximab (Erbitux®), iressa, OSI-774, CI-1033, EKB-569, GW-2016, E1.1, E2.4, E2.5, E6.2, E6.4, E2.11, E6.3 or E7.6.3, 7H-pyrrolo-[2,3-d]pyrimidine derivatives which are e.g. disclosed in WO03013541, erlotinib, gefitinib. Erlotinib can be administered in the form as it is marketed, e.g. TARCEVA®, and gefitinib as IRESSA®, human monoclonal antibodies against the epidermal growth factor receptor including ABX-EGFR.
xxx. an EGFR, PDGFR tyrosine kinase inhibitor; such as EGFR kinase inhibitors, e.g. zalutumumab, tyrphostin 23, tyrphostin 25, tyrphostin 47, tyrphostin 51 and tyrphostin AG 825; 2-propenamide, 2-cyano-3-(3,4-dihydroxyphenyl)-N-phenyl-(2E); tyrphostin Ag 1478; lavendustin A; 3-pyridineacetonitrile, α-[(3,5-dichlorophenyl)methylene]-, (αZ); an example of an EGFR, PDGFR tyrosine kinase inhibitor e.g. includes tyrphostin 46. PDGFR tyrosine kinase inhibitor including tyrphostin 46. Targets of an EGFR kinase inhibitor include guanylyl cyclase (GC-C) HER2, EGFR, PTK and tubulin.
xxxi. a farnesyltransferase inhibitor; which targets, decreases or inhibits the Ras protein; such as a-hydroxyfarnesylphosphonic acid; butanoic acid, 2-[[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-, 1-methylethyl ester, (2S); manumycin A; L-744,832 or DK8G557, tipifamib (R115777), SCH66336 (Ionafarnib), BMS-214662,
xxxii. a Flk-1 kinase inhibitor, which targets, decreases or inhibits Flk-1 tyrosine kinase activity; such as 2-propenamide, 2-cyano-3-[4-hydroxy-3,5-bis(1-methylethyl)phenyl]-N-(3-phenylpropyl)-(2E). A target of a Flk-1 kinase inhibitor includes, but is not limited to, KDR.
xxxiii. a Glycogen synthase kinase-3 (GSK3) inhibitor; which targets, decreases or inhibits glycogen synthase kinase-3 (GSK3); such as indirubin-3′-monooxime. Glycogen Synthase Kinase-3 (GSK-3; tau protein kinase I), a highly conserved, ubiquitously expressed serine/threonine protein kinase, is involved in the signal transduction cascades of multiple cellular processes. which is a protein kinase that has been shown to be involved in the regulation of a diverse array of cellular functions, including protein synthesis, cell proliferation, cell differentiation, microtubule assembly/disassembly, and apoptosis.
xxxiv. a histone deacetylase (HDAC) inhibitor; which inhibits the histone deacetylase and which possess anti-proliferative activity; such as compounds disclosed in WO0222577, especially N-hydroxy-3-[4-[[(2-hydroxyethyl)[2-(1H-indol-3-yl)ethyl]-amino]methyl]phenyl]-2E-2-propenamide, and N-hydroxy-3-[(4-[[[2-(2-methyl-1H-indol-3-yl)-ethyl]-amino]methyl]phenyl]-2E-2-propenamide and pharmaceutically acceptable salts thereof; suberoylanilide hydroxamic acid (SAHA); [4-(2-amino-phenylcarbamoyl)-benzyl]-carbamic acid pyridine-3-ylmethyl ester and derivatives thereof; butyric acid, pyroxamide, trichostatin A, oxamflatin, apicidin, depsipeptide; depudecin; trapoxin, HC toxin, which is also known as cyclo[L-alanyl-D-alanyl-(□S,2S)-□-amino-□-oxooxiraneoctanoyl-D-prolyl] (9Cl); sodium phenylbutyrate, suberoyl bis-hydroxamic acid; Trichostatin A, BMS-27275, pyroxamide, FR-901228, valproic acid.
xxxv. a HSP90 inhibitor; which targets, decreases or inhibits the intrinsic ATPase activity of HSP90; degrades, targets, decreases or inhibits the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, e.g., 17-allylamino, 17-demethoxygeldanamycin (17AAG), a geldanamycin derivative; other geldanamycin-related compounds; radicicol and HDAC inhibitors. Other examples of an HSP90 inhibitor include geldanamycin, 17-demethoxy-17-(2-propenylamino). Potential indirect targets of an HSP90 inhibitor include FLT3, BCR-ABL, CHK1, CYP3A5*3 and/or NQ01*2. Nilotinib is an example of an BCR-ABL tyrosine kinase inhibitor.
xxxvi. a I-kappa B-alpha kinase inhibitor (IKK); which targets, decreases or inhibits NF-kappaB, such as 2-propenenitrile, 3-[(4-methylphenyl)sulfonyl]-(2E).
xxxvii. an insulin receptor tyrosine kinase inhibitor; which modulates the activities of phosphatidylinositol 3-kinase, microtubule-associated protein, and S6 kinases; such as hydroxyl-2-naphthalenylmethylphosphonic acid, LY294002.
xxxviii. a c-Jun N-terminal kinase (JNK) kinase inhibitor; which targets, decreases or inhibits Jun N-terminal kinase; such as pyrazoleanthrone and/or epigallocatechin gallate. Jun N-terminal kinase (JNK), a serine-directed protein kinase, is involved in the phosphorylation and activation of c-Jun and ATF2 and plays a significant role in metabolism, growth, cell differentiation, and apoptosis. A target for a JNK kinase inhibitor includes, but is not limited to, DNMT.
xxxix a microtubule binding agent; which acts by disrupting the microtubular network that is essential for mitotic and interphase cellular function; such as vinca alkaloids, e.g. vinblastine, vinblastine sulfate; vincristine, vincristine sulfate; vindesine; vinorelbine; taxanes, such as taxanes, e.g. docetaxel; paclitaxel; discodermolides; colchicine, epothilones and derivatives thereof, e.g. epothilone B or a derivative thereof. Paclitaxel is marketed as TAXOL®; docetaxel as TAXOTERE®; vinblastine sulfate as VINBLASTIN R.P®; and vincristine sulfate as FARMISTIN®. Also included are the generic forms of paclitaxel as well as various dosage forms of paclitaxel. Generic forms of paclitaxel include, but are not limited to, betaxolol hydrochloride. Various dosage forms of paclitaxel include, but are not limited to albumin nanoparticle paclitaxel marketed as ABRAXANE®; ONXOL®, CYTOTAX®. Discodermolide can be obtained, e.g., as disclosed in U.S. Pat. No. 5,010,099. Also included are Epotholine derivatives which are disclosed in U.S. Pat. No. 6,194,181, WO98/0121, WO9825929, WO9808849, WO9943653, WO9822461 and WO0031247. Especially preferred are Epotholine A and/or B.
xl. a mitogen-activated protein (MAP) kinase-inhibitor; which targets, decreases or inhibits Mitogen-activated protein, such as benzenesulfonamide, N-[2-[[[3-(4-chlorophenyl)-2-propenyl]nethyl]amino]methyl]phenyl]-N-(2-hydroxyethyl)-4-methoxy. The mitogen-activated protein (MAP) kinases are a group of protein serine/threonine kinases that are activated in response to a variety of extracellular stimuli and mediate signal transduction from the cell surface to the nucleus. They regulate several physiological and pathological cellular phenomena, including inflammation, apoptotic cell death, oncogenic transformation, tumor cell invasion, and metastasis.
xli. a MDM2 inhibitor; which targets, decreases or inhibits the interaction of MDM2 and the p53 tumor suppressor; such as trans-4-iodo, 4′-boranyl-chalcone.
xlii. a MEK inhibitor; which targets, decreases or inhibits the kinase activity of MAP kinase MEK; such as sorafenib, e.g. Nexavar® (sorafenib tosylate), butanedinitrile, bis[amino[2-aminophenyl)thio]methylene]. A target of a MEK inhibitor includes, but is not limited to ERK. An indirect target of a MEK inhibitor includes, but is not limited to, cyclin D1.
xliii: a matrix metalloproteinase inhibitor (MMP) inhibitor; which targets, decreases or inhibits a class of protease enzyme that selectively catalyze the hydrolysis of polypeptide bonds including the enzymes MMP-2 and MMP-9 that are involved in promoting the loss of tissue structure around tumors and facilitating tumor growth, angiogenesis, and metastasissuch as actinonin, which is also known as butanediamide, N-4-hydroxy-N1-[(1S)-1-[[(2S)-2-(hydroxymethyl)-1-pyrrolidinyl]carbonyl]-2-methylpropyl]-2-pentyl-, (2R)-(9Cl); epigallocatechin gallate; collagen peptidomimetic and non-peptidomimetic inhibitors; tetracycline derivatives, e.g., hydroxamate peptidomimetic inhibitor batimastat; and its orally-bioavailable analogue marimastat, prinomastat, metastat, neovastat, tanomastat, TAA211, BMS-279251, BAY 12-9566, MMI270B or AAJ996. A target of a MMP inhibitor includes, but is not limited to, polypeptide deformylase.
xliv. a NGFR tyrosine-kinase-inhibitor; which targets, decreases or inhibits nerve growth factor dependent p140^{c-trk} tyrosine phosphorylation; such as tyrphostin AG 879. Targets of a NGFR tyrosine-kinase-inhibitor include, but are not limited to, HER2, FLK1, FAK, TrkA, and/or TrkC. An indirect target inhibits expression of RAF1.
xlv. a p38 MAP kinase inhibitor, including a SAPK2/p38 kinase inhibitor; which targets, decreases or inhibits p38-MAPK, which is a MAPK family member, such as phenol, 4-[4-(4-fluorophenyl)-5-(4-pyridinyl)-1H-imidazol-2-yl]. An example of a SAPK2/p38 kinase inhibitor includes, but is not limited to, benzamide, 3-(dimethylamino)-N-3-[(4-hydroxybenzoyl)amino)-4-methylphenyl]. A MAPK family member is a serine/threonine kinase activated by phosphorylation of tyrosine and threonine residues. This kinase is phosphorylated and activated by many cellular stresses and inflammatory stimuli, thought to be involved in the regulation of important cellular responses such as apoptosis and inflammatory reactions.
xlvi. a p56 tyrosine kinase inhibitor; which targets, decreases or inhibits p56 tyrosine kinase, which is an enzyme that is a lymphoid-specific src family tyrosine kinase critical for T-cell development and activation; such as damnacanthal, which is also known as 2-anthracenecarboxaldehyde, 9,10-dihydro-3-hydroxy-1-methoxy-9,10-dioxo, Tyrphostin 46. A target of a p56 tyrosine kinase inhibitor includes, but is not limited to, Lck. Lck is associated with the cytoplasmic domains of CD4, CD8 and the beta-chain of the IL-2 receptor, and is thought to be involved in the earliest steps of TCR-mediated T-cell activation.
xlvii. a PDGFR tyrosine kinase inhibitor; targeting, decreasing or inhibiting the activity of the C-kit receptor tyrosine kinases (part of the PDGFR family), such as targeting, decreasing or inhibiting the activity of the c-Kit receptor tyrosine kinase family, especially inhibiting the c-Kit receptor. Examples of targets of a PDGFR tyrosine kinase inhibitor includes, but are not limited to PDGFR, FLT3 and/or c-KIT; such as tyrphostin AG 1296; tyrphostin 9; 1,3-butadiene-1,1,3-tricarbonitrile, 2-amino-4-(1H-indol-5-yl); N-phenyl-2-pyrimidine-amine derivative, e.g. imatinib, IRESSA®. PDGF plays a central role in regulating cell proliferation, chemotaxis, and survival in normal cells as well as in various disease states such as cancer, atherosclerosis, and fibrotic disease. The PDGF family is composed of dimeric isoforms (PDGF-AA, PDGF-BB, PDGF-AB, PDGF-CC, and PDGF-DD), which exert their cellular effects by differentially binding to two receptor tyrosine kinases. PDGFR-α and PDGFR-1 have molecular masses of ˜170 and 180 kDa, respectively.
xlviii. a phosphatidylinositol 3-kinase inhibitor; which targets, decreases or inhibits PI 3-kinase; such as wortmannin, which is also known as 3H-Furo[4,3,2-de]indeno[4,5-h]-2-benzopyran-3,6,9-trione, 11-(acetyloxy)-1,6b,7,8,9a,10,11,11b-octahydro-1-(methoxymethyl)-9a,11b-dimethyl-, (1S,6bR,9aS,11R,11bR)— (9Cl); 8-phenyl-2-(morpholin-4-yl)-chromen-4-one; quercetin, quercetin dihydrate. PI 3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation. An example of a target of a phosphatidylinositol 3-kinase inhibitor includes, but is not limited to, Pi3K.
xlix. a phosphatase inhibitor; which targets, decreases or inhibits phosphatase; such as cantharidic acid; cantharidin; and L-leucinamide, N-[4-(2-carboxyethenyl)benzoyl]glycyl-L-α-glutamyl-(E). Phosphatases remove the phosphoryl group and restore the protein to its original dephosphorylated state. Hence, the phosphorylation-dephosphorylation cycle can be regarded as a molecular “on-off” switch.
l. a platinum agent; which contains platinum and inhibit DNA synthesis by forming interstrand and intrastrand cross-linking of DNA molecules; such as carboplatin; cisplatin; oxaliplatin; cisplatinum; satraplatin and platinum agents such as ZD0473, BBR3464. Carboplatin can be administered, e.g., in the form as it is marketed, e.g. CARBOPLAT®; and oxaliplatin as ELOXATIN®.
li. a protein phosphatase inhibitor, including a PP1 and PP2 inhibitor and a tyrosine phosphatase inhibitor; which targets, decreases or inhibits protein phosphatase. Examples of a PP1 and PP2A inhibitor include cantharidic acid and/or cantharidin. Examples of a tyrosine phosphatase inhibitor include, but are not limited to, L-P-bromotetramisole oxalate; 2(5H)-furanone, 4-hydroxy-5-(hydroxymethyl)-3-(1-oxohexadecyl)-, (5R); and benzylphosphonic acid.
   The term “a PP1 or PP2 inhibitor”, as used herein, relates to a compound which targets, decreases or inhibits Ser/Thr protein phosphatases. Type I phosphatases, which include PP1, can be inhibited by two heat-stable proteins known as Inhibitor-1 (I-1) and Inhibitor-2 (I-2). They preferentially dephosphorylate a subunit of phosphorylase kinase. Type II phosphatases are subdivided into spontaneously active (PP2A), CA²⁺-dependent (PP2B), and Mg²⁺-dependent (PP2C) classes of phosphatases.
   The term “tyrosine phosphatase inhibitor”, as used here, relates to a compounds which targets, decreases or inhibits tyrosine phosphatase. Protein tyrosine phosphatases (PTPs) are relatively recent additions to the phosphatase family. They remove phosphate groups from phosphorylated tyrosine residues of proteins. PTPs display diverse structural features and play important roles in the regulation of cell proliferation, differentiation, cell adhesion and motility, and cytoskeletal function. Examples of targets of a tyrosine phosphatase inhibitor include, but are not limited to, alkaline phosphatase (ALP), heparanase, PTPase, and/or prostatic acid phosphatase.
lii. a PKC inhibitor and a PKC delta kinase inhibitor: The term “a PKC inhibitor”, as used herein, relates to a compound which targets, decreases or inhibits protein kinase C as well as its isozymes. Protein kinase C (PKC), a ubiquitous, phospholipid-dependent enzyme, is involved in signal transduction associated with cell proliferation, differentiation, and apoptosis. Examples of a target of a PKC inhibitor include, but are not limited to, MAPK and/or NF-kappaB. Examples of a PKC inhibitor include, but are not limited to, 1-H-pyrrolo-2,5-dione, 3-[1-[3-(dimethylamino)propyl]-1H-indol-3-yl]-4-(1H-indol-3-yl); bisindolylmaleimide IX; sphingosine, which is known as 4-octadecene-1,3-diol, 2-amino-, (2S,3R,4E)-(9Cl); staurosporine, which is known as 9,13-Epoxy-1H,9H-diindolo[1,2,3-gh:3′,2′,1′-Im]pyrrolo[3,4-j][1,7]benzodiazonin-1-one, staurosporine derivatives such as disclosed in EP0296110, e.g. midostaurin; 2,3,10,11,12,13-hexahydro-10-methoxy-9-methyl-1′-(methylamino)-, (9S,10R,11R,13R)-(9Cl); tyrphostin 51; and hypericin, which is also known as phenanthro[1,10,9,8-opqra]perylene-7,14-dione, 1,3,4,6,8,13-hexahydroxy-10,11-dimethyl-, stereoisomer (6Cl,7Cl,8Cl,9Cl), UCN-01,safingol, BAY 43-9006, bryostatin 1, perifosine; Ilmofosine; RO 318220 and RO 320432; GO 6976; Isis 3521; LY333531/LY379196. The term “a PKC delta kinase inhibitor”, as used herein, relates to a compound which targets, decreases or inhibits the delta isozymes of PKC. The delta isozyme is a conventional PKC isozymes and is Ca²⁺-dependent. An example of a PKC delta kinase inhibitor includes, but is not limited to, Rottlerin, which is also known as 2-Propen-1-one, 1-[6-[(3-acetyl-2,4,6-trihydroxy-5-methylphenyl)methyl]-5,7-dihydroxy-2,2-dimethyl-2H-1-benzopyran-8-yl]-3-phenyl-, (2E)-(9Cl).
liii. a polyamine synthesis inhibitor; which targets, decreases or inhibits polyamines spermidine; such as DMFO, which is also known as (−)-2-difluoromethylornithine; N1,N12-diethylspermine 4HCl. The polyamines spermidine and spermine are of vital importance for cell proliferation, although their precise mechanism of action is unclear. Tumor cells have an altered polyamine homeostasis reflected by increased activity of biosynthetic enzymes and elevated polyamine pools.
liv. a proteosome inhibitor; which targets, decreases or inhibits proteasome, such as aclacinomycin A; gliotoxin; PS-341; MLN 341; bortezomib; velcade. Examples of targets of a proteosome inhibitor include, but are not limited to, O(2)(−)-generating NADPH oxidase, NF-kappaB, and/or farnesyltransferase, geranyltransferase I.
lv. a PTP1B inhibitor; which targets, decreases or inhibits PTP1B, a protein tyrosine kinase inhibitor; such as L-leucinamide, N-[4-(2-carboxyethenyl)benzoyl]glycyl-L-α-glutamyl-, (E).
lvi. a protein tyrosine kinase inhibitor including a SRC family tyrosine kinase inhibitor; a Syk tyrosine kinase inhibitor; and a JAK-2 and/or JAK-3 tyrosine kinase inhibitor; The term “a protein tyrosine kinase inhibitor”, as used herein, relates to a compound which targets, decreases or inhibits protein tyrosine kinases. Protein tyrosine kinases (PTKs) play a key role in the regulation of cell proliferation, differentiation, metabolism, migration, and survival. They are classified as receptor PTKs and non-receptor PTKs. Receptor PTKs contain a single polypeptide chain with a transmembrane segment. The extracellular end of this segment contains a high affinity ligand-binding domain, while the cytoplasmic end comprises the catalytic core and the regulatory sequences. Examples of targets of a tyrosine kinase inhibitor include, but are not limited to, ERK1, ERK2, Bruton's tyrosine kinase (Btk), JAK2, ERK ½, PDGFR, and/or FLT3. Examples of indirect targets include, but are not limited to, TNFalpha, NO, PGE2, IRAK, iNOS, ICAM-1, and/or E-selectin. Examples of a tyrosine kinase inhibitor include, but are not limited to, tyrphostin AG 126; tyrphostin Ag 1288; tyrphostin Ag 1295; geldanamycin; and genistein.
   Non-receptor tyrosine kinases include members of the Src, Tec, JAK, Fes, Abl, FAK, Csk, and Syk families. They are located in the cytoplasm as well as in the nucleus. They exhibit distinct kinase regulation, substrate phosphorylation, and function. Deregulation of these kinases has also been linked to several human diseases. The term “a SRC family tyrosine kinase inhibitor”, as used herein, relates to a compound which targets, decreases or inhibits SRC. Examples of a SRC family tyrosine kinase inhibitor include, but are not limited to, PP1, which is also known as 1H-pyrazolo[3,4-d]pyrimidin-4-amine, 1-(1,1-dimethylethyl)-3-(1-naphthalenyl)-(9Cl); and PP2, which is also known as 1H-Pyrazolo[3,4-d]pyrimidin-4-amine, 3-(4-chlorophenyl)-1-(1,1-dimethylethyl)-(9Cl).
   The term “a Syk tyrosine kinase inhibitor”, as used herein, relates to a compound which targets, decreases or inhibits Syk. Examples of targets for a Syk tyrosine kinase inhibitor include, but are not limited to, Syk, STAT3, and/or STAT5. An example of a Syk tyrosine kinase inhibitor includes, but is not limited to, piceatannol, which is also known as 1,2-benzenediol, 4-[(1E)-2-(3,5-dihydroxyphenyl)ethenyl]-(9Cl). The term “a Janus (JAK-2 and/or JAK-3) tyrosine kinase inhibitor”, as used herein, relates to a compound which targets, decreases or inhibits janus tyrosine kinase. Janus tyrosine kinase inhibitor are shown anti-leukemic agents with anti-thrombotic, anti-allergic and immunosuppressive properties. Targets of a JAK-2 and/or JAK-3 tyrosine kinase inhibitor include, but are not limited to, JAK2, JAK3, STAT3. An indirect target of an JAK-2 and/or JAK-3 tyrosine kinase inhibitor includes, but is not limited to CDK2. Examples of a JAK-2 and/or JAK-3 tyrosine kinase inhibitor include, but are not limited to, Tyrphostin AG 490; and 2-naphthyl vinyl ketone. Compounds which target, decrease or inhibit the activity of c-Abl family members and their gene fusion products, e.g. include PD180970; AG957; or NSC 680410.
lvii. a retinoid; which target, decrease or inhibit retinoid dependent receptors; such as isotretinoin, tretinoin, alitretinoin, bexarotene, e.g. including an agent which interact with retinoic acid responsive elements on DNA, such as isotretinoin (13-cis-retinoic acid).
lviii. a RNA polymerase II elongation inhibitor; which targets, decreases or inhibits insulin-stimulated nuclear and cytosolic p70S6 kinase in CHO cells; targets, decreases or inhibits RNA polymerase II transcription, which may be dependent on casein kinase II; and targets, decreases or inhibits germinal vesicle breakdown in bovine oocytes; such as 5,6-dichloro-1-beta-D-ribofuranosylbenzimidazole.
ivix. a serine/threonine kinase inhibitor; which inhibits serine/threonine kinases; such as 2-aminopurine. An example of a target of a serine/threonine kinase inhibitor includes, but is not limited to, dsRNA-dependent protein kinase (PKR). Examples of indirect targets of a serine/threonine kinase inhibitor include, but are not limited to, MCP-1, NF-kappaB, eIF2alpha, COX2, RANTES, IL8, CYP2A5, IGF-1, CYP2B1, CYP2B2, CYP2H1, ALAS-1, HIF-1, erythropoietin, and/or CYP1A1.
lx. a sterol biosynthesis inhibitor; which inhibits the biosynthesis of sterols such as cholesterol; such as terbinadine. Examples of targets for a sterol biosynthesis inhibitor include, but are not limited to, squalene epoxidase, and CYP2D6.
lxi. a topoisomerase inhibitor; including a topoisomerase I inhibitor and a topoisomerase II inhibitor. Examples of a topoisomerase I inhibitor include, but are not limited to, topotecan, gimatecan, irinotecan, camptothecan and its analogues, 9-nitrocamptothecin and the macromolecular camptothecin conjugate PNU-166148 (compound A1 in WO9917804); 10-hydroxycamptothecin e.g. the acetate salt; idarubicin, e.g. the hydrochloride; irinotecan, e.g. the hydrochloride; etoposide; teniposide; topotecan, topotecan hydrochloride; doxorubicin; epirubicin, epirubicin hydrochloride; 4′-epidoxorubicin, mitoxantrone, mitoxantrone, e.g. the hydrochloride; daunorubicin, daunorubicin hydrochloride, valrubicin, dasatinib (BMS-354825). irinotecan can be administered, e.g., in the form as it is marketed, e.g., under the trademark CAMPTOSAR®. Topotecan can be administered, e.g., in the form as it is marketed, e.g., under the trademark HYCAMTIN®. The term “topoisomerase II inhibitor”, as used herein, includes, but is not limited to, the anthracyclines, such as doxorubicin, including liposomal formulation, e.g., CAELYX®, daunorubicin, including liposomal formulation, e.g., DAUNOSOME®, epirubicin, idarubicin and nemorubicin; the anthraquinones mitoxantrone and losoxantrone; and the podophillotoxines etoposide and teniposide. Etoposide is marketed as ETOPOPHOS®; teniposide as VM 26-BRISTOL®; doxorubicin as ADRIBLASTIN® or ADRIAMYCIN®; epirubicin as FARMORUBICIN® idarubicin as ZAVEDOS®; and mitoxantrone as NOVANTRON®.
lxii. VEGFR tyrosine kinase inhibitor; which targets, decreases and/or inhibits the known angiogenic growth factors and cytokines implicated in the modulation of normal and pathological angiogenesis. The VEGF family (VEGF-A, VEGF-B, VEGF-C, VEGF-D) and their corresponding receptor tyrosine kinases [VEGFR-1 (Flt-1), VEGFR-2 (Flk-1, KDR), and VEGFR-3 (Flt-4)] play a paramount and indispensable role in regulating the multiple facets of the angiogenic and lymphangiogenic processes. An example of a VEGFR tyrosine kinase inhibitor includes 3-(4-dimethylaminobenzylidenyl)-2-indolinone. Compounds which target, decrease or inhibit the activity of VEGFR are especially compounds, proteins or antibodies which inhibit the VEGF receptor tyrosine kinase, inhibit a VEGF receptor or bind to VEGF, and are in particular those compounds, proteins or monoclonal antibodies generically and specifically disclosed in WO9835958, e.g. 1-(4-chloroanilino)-4-(4-pyridylmethyl) phthalazine or a pharmaceutical acceptable salt thereof, e.g. the succinate, or in WO0009495, WO0027820, WO0059509, WO9811223, WO0027819 and EP0769947; e.g. those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999; in WO0037502 and WO9410202; Angiostatin, described by M. S. O'Reilly et al, Cell 79, 1994, 315-328; Endostatin described by M. S. O'Reilly et al, Cell 88, 1997, 277-285; anthranilic acid amides; ZD4190; ZD6474 (vandetanib); SU5416; SU6668, AZD2171 (Recentin®); or anti-VEGF antibodies, such as anti-VEGF-alpha antibody tanibizumab (Lucentis®), or anti-VEGF receptor antibodies, e.g. RhuMab (bevacizumab, Avastin®). By antibody is meant intact monoclonal antibodies, polyclonal antibodies, multispecific antibodies formed from at least 2 intact antibodies, and antibodies fragments so long as they exhibit the desired biological activity. an example of an VEGF-R2 inhibitor e.g. includes axitinib,
lxiii. a gonadorelin agonist, such as abarelix, goserelin, goserelin acetate,
lxiv. a compound which induce cell differentiation processes, such as retinoic acid, alpha-, gamma- or 8-tocopherol or alpha-, gamma- or 8-tocotrienol.
lxv. a bisphosphonate, e.g. including etridonic, clodronic, tiludronic, pamidronic, alendronic, ibandronic, risedronic and zoledronic acid.
lxvi. a heparanase inhibitor which prevents heparan sulphate degradation, e.g. PI-88,
lxvii. a biological response modifier, preferably alymphokine or interferons, e.g. interferon alpha,
lxviii. a telomerase inhibitor, e.g. telomestatin,
lxix. mediators, such as inhibitors of catechol-O-methyltransferase, e.g. entacapone,
lxx: ispinesib, permetrexed (Alimta®), sunitinib (SU11248), diethylstilbestrol (DES), BMS224818 (LEA29Y), vatanalib,
lxxi somatostatin or a somatostatin analogue, such as octreotide (Sandostatin® or Sandostatin LAR®).
lxxii. Growth Hormone—Receptor Antagonists, such as pegvisomant, filgrastim or pegfilgrastim, or interferon alpha:
lxxiii. monoclonal antibodies, e.g. useful for leukemia (AML) treatment, such as alemtuzumab (Campath®), rituximab/Rituxan®), gemtuzumab, (ozogamicin, Mylotarg®), epratuzumab.
lxxiv. altretamine, amsacrine, asparaginase (Elspar®), denileukin diftitox, masoprocol, pegaspargase, gemtuzumab (MYLOTARG®),
lxxv. a phosphodiesterase inhibitor, e.g. anagrelide (Agrylin®, Xagrid®).
lxxvi. a cancer vaccine, such as MDX-1379.
lxxvii. an immunosuppressive monoclonal antibody, e.g., monoclonal antibodies to leukocyte receptors,
   e.g. CD20, such as rituximab (Rituxan®, ibritumomab tiuxetan conjugated to ¹¹¹In or ⁹⁰Y (Zevalin®), ¹³¹I tositumumab (Bexxar®), ofatumumab, ocrelizumab, hA20 (Immunomedics),
   CD22, such as epratuzumab, inotizumab ozogamicin (CMC544), CAT-3888,
   CD33, such as gemtuzumab (Mylotarg®,
   CD52, e.g. alemtuzumab (Campath-I®),
   or their ligands,
   CD11a, e.g. efalizumab (Raptiva®),
   CD3, e.g. visillzumab,
lxxviii. antibodies against carcinoembryonic antigen (CEA), e.g. lapetuzumab, e.g. lapetuzumab-yttrium90, KSB-303, MFECPI, MFE-23,

Cancer treatment optionally in combination with an anticancer drug may be associated with radiotherapy, e.g. including DOTATATE therapy, such as Y⁹⁰-DOTATATE therapy.

Cancer treatment may also be associated with vitamin or vitamin derivative (e.g. Leucovorin®) treatment.

Anti-cancer drugs, e.g. for the treatment of breast cancer, e.g. may be used in combination with Abraxane® which may improve the release of drugs, and even may enhance the drug benefit, e.g. such as in case of administration of paclitaxel in combination with Abraxane®. (wherein Abraxane® combines the drug paclitaxel with the protein albumin, which turns into a nanoparticle when injected into the bloodstream allowing a greater concentration of the drug in the tumor and starving the malignant cells of the nutrients they need to grow).

If the compounds of the present invention are administered in combination with other drugs dosages of the co-administered second drug will of course vary depending on the type of co-drug employed, on the specific drug employed, on the condition being treated, as in case of a compound of the present invention. In general dosages similar than those as provided by the second drug supplier may be appropriate

The chemical names of the compounds of the present invention as indicated herein are copied from ISIS, version 2.5 (AutoNom 2000 Name). Chemical names of second drug substances and other substances may be derived from the Internet, e.g. via a search program such as the SCI FINDER.

In another aspect the present invention provides a compound of formula

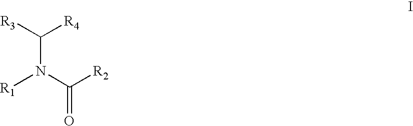

wherein
R₁ is aryl, cyclohexyl or heterocyclyl, or (C₁₋₄)alkyl substituted by aryl, cyclohexyl or heterocyclyl,
R₂ is heterocyclyl,
R₃ is branched (C₃₋₁₂)alkyl, aryl, cyclohexyl or heterocyclyl, or (C₁₋₄)alkyl substituted by aryl, cyclohexyl or heterocydyl, and
R₄ is H or alkyl.

In another aspect the present invention provides a compound of formula

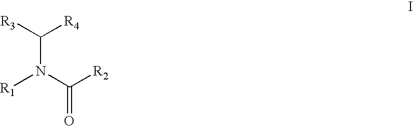

wherein
R₁ is aryl, cyclohexyl or heterocyclyl, or (C₁₋₄)alkyl substituted by aryl, cyclohexyl or heterocyclyl,
R₂ is heterocyclyl,
R₃ is alkyl, aryl, cyclohexyl or heterocyclyl, or (C₁₋₄)alkyl substituted by aryl, cyclohexyl or heterocyclyl,
R₄ is H or alkyl, or
R₃ and R₄ together with the carbon atom to which they are attached are cycloalkyl fused with phenyl.

In the following examples all temperatures indicated are in degree Celsius (° C.).

The following abbreviations are used

EtOAc ethyl acetate

RT room temperature

### EXAMPLE 1

### N-(3,5-dichloro-phenyl)-N-(2-methoxy-benzyl)-3-methyl-isonicotinamide Compound of EX 2 in Table 1 Below

2.25 g of 2-methoxybenzaldehyde in 10 ml of dry CH₂Cl₂ are treated with 3.18 g of 3,5-dichloroaniline and 6.93 g of sodium triacetoxyborohydride and the mixture obtained is stirred at RT until TLC analysis shows consumption of starting material. To the mixture obtained a saturated aqueous solution of NaHCO₃ is slowly added until no further gas evolution occurs. A two phase system is formed and the aqueous layer is separated and washed with EtOAc. The combined organic layers are dried, solvent is removed under reduced pressure and (3,5-dichloro-phenyl)-(2-methoxy-benzyl)-amine is obtained. 150 mg of 3-methyl-isonicotinic acid in 10 ml of dry CH₂Cl₂ are treated with 217 μl of 1-chloro-N,N,2-trimethyl-1-propenylamine and the mixture obtained is stirred at RT until TLC analysis shows consumption of starting material. To the mixture obtained 339 mg of (3,5-dichloro-phenyl)-(2-methoxy-benzyl)-amine and 460 μl of triethylamine are added and the mixture obtained is stirred at RT until TLC analysis shows no further reaction progress. The mixture obtained is diluted with EtOAc and treated with a saturated aqueous solution of NaHCO₃. The aqueous layer is separated and washed with EtOAc. A two phase system is formed and the aqueous layer is separated and washed with EtOAc. The combined organic layers are dried, solvent is removed under reduced pressure and N-(3,5-dichloro-phenyl)-N-(2-methoxy-benzyl)-3-methyl-isonicotinamide is obtained.

### EXAMPLE 2

### N-(3,5-dichloro-phenyl)-N-[1-(2-methoxy-phenyl)-ethyl]-3-methyl-isonicotinamide Compound of EX 118 in Table 1

Under an atmosphere of argon, 2.11 g Of 3,5-dichloroaniline in 50 ml of dry CH₂Cl₂ are treated with 1.96 g of 2-methoxyacetophenone and 3.96 g of triethylamine. To the mixture obtained 6.5 ml of a solution of titanium tetrachloride (1.0 M in CH₂Cl₂) is added dropwise and the mixture obtained is stirred at RT overnight. To the mixture obtained 39 ml of a solution of sodium cyanoborohydride (1.0 M in methanol) is added and the mixture is stirred for 1 hour. The pH of the mixture is adjusted to ca. 13 by addition of NaOH (1.0 M in water) and EtOAc is added. A two phase system is formed and the aqueous layer is separated and washed with EtOAc. The combined organic layers are dried and solvent is removed under reduced pressure. (3,5-dichloro-phenyl)-[1-(2-methoxy-phenyl)-ethyl]-amine is obtained. 136 mg of 3-methyl-isonicotinic acid in 3 ml of dry CH₂Cl₂ are treated with 140 μl of 1-chloro-N,N,2-trimethyl-1-propenylamine and the mixture obtained is stirred at RT until TLC analysis shows consumption of starting material. To the mixture obtained 95 mg of (3,5-dichloro-phenyl)-[1-(2-methoxy-phenyl)-ethyl]-amine, 40 mg of N,N-dimethyl-4-aminopyridine and 97 mg of triethylamine are added. The mixture obtained is stirred at RT until TLC analysis shows no further reaction progress. The mixture obtained is diluted with EtOAc and treated with a saturated aqueous solution of NaHCO₃. A two phase system is formed and the aqueous layer is separated and washed with EtOAc. The combined organic layers are dried, solvent is removed under reduced pressure and N-(3,5-dichloro-phenyl)-N-[1-(2-methoxy-phenyl)-ethyl]-3-methyl-isonicotinamide is obtained.

Analogously to a method as described in Examples 1 and 2, but using appropriate starting materials (intermediates) compounds of formula

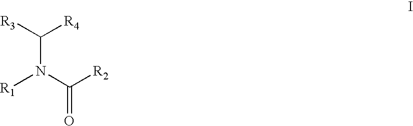

wherein R₁, R₂, R₃ and R₄ are as set out in TABLE I below, are obtained:

TABLE 1

| EX | R₁ | R₂ | R₃ | R₄ | MS |
|---|---|---|---|---|---|
| 1 | 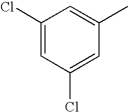 | 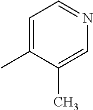 | 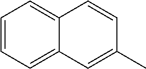 | H | 443.10/ 445.12 |
| 2 | 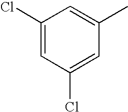 | 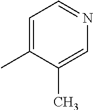 | 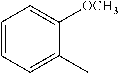 | H | 423.05/ 425.03 |
| 3 | 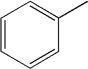 | 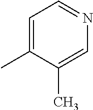 | 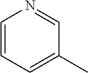 | H | 326.13 |
| 4 | 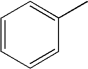 | 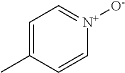 | 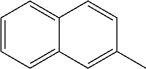 | H | 391.13 |
| 5 | 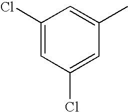 | 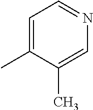 | 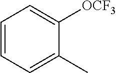 | H | 455.07/ 457.08 |
| 6 | 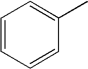 | 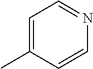 | 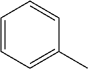 | H | 289.07 |
| 7 | 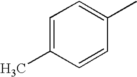 | 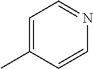 | 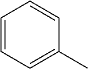 | H | 325.11 |
| 8 | 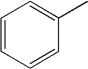 | 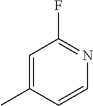 | 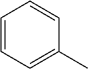 | H | 307.05 |
| 9 | 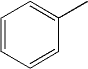 | 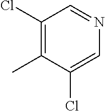 | 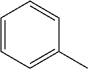 | H | 356.90/ 358.91 |
| 10 | 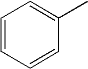 | 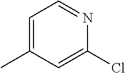 | 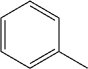 | H | 323.04/ 324.99 |
| 11 | 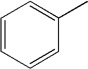 | 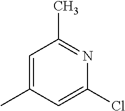 | 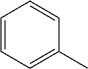 | H | 337.01/ 339.02 |
| 12 | 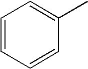 | 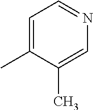 | 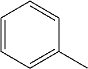 | H | 303.09 |
| 13 | 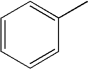 | 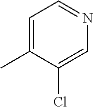 | 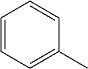 | H | 323.04/ 324.99 |
| 14 | 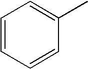 | 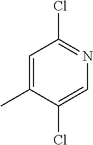 | 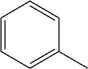 | H | 356.96/ 358.91 |
| 15 | 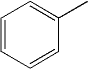 | 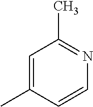 | 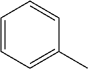 | H | 303.09 |
| 16 | 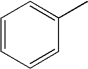 | 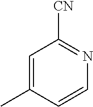 | 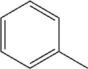 | H | 314.04 |
| 17 | 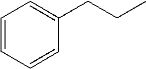 | 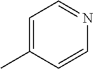 | 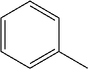 | H | 317.1 |
| 18 | 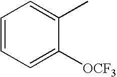 | 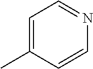 | 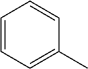 | H | 373 |
| 19 | 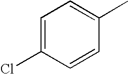 | 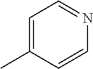 | 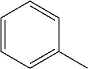 | H | 323.2 |
| 20 | 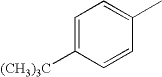 | 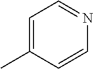 | 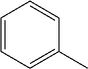 | H | 345.1 |
| 21 | 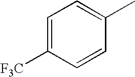 | 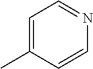 | 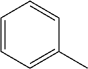 | H | 357.1 |
| 22 | 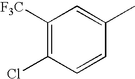 | 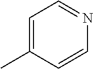 | 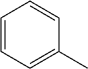 | H | 391.1 |
| 23 | 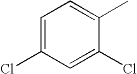 | 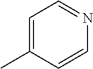 | 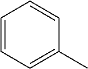 | H | 357 |
| 24 | 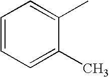 | 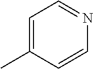 | 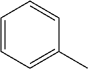 | H | 303.1 |
| 25 | 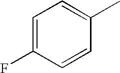 | 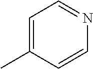 | 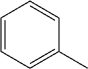 | H | 307.1 |
| 26 | 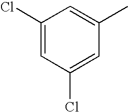 | 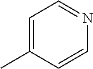 | 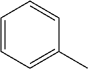 | H | 357 |
| 27 | 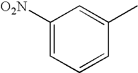 | 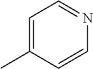 | 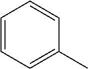 | H | 334.1 |
| 28 | 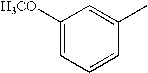 | 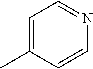 | 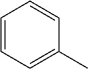 | H | 319.1 |
| 29 | 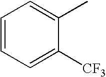 | 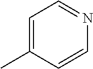 | 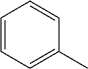 | H | 357 |
| 30 | 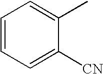 | 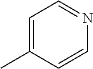 | 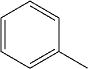 | H | 314 |
| 31 | 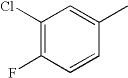 | 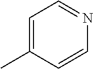 | 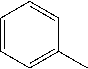 | H | 341 |
| 32 | 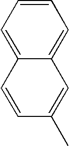 | 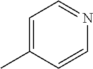 | 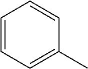 | H | 339.1 |
| 33 | 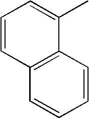 | 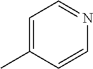 | 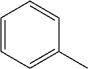 | H | 339.1 |
| 34 | 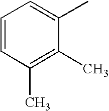 | 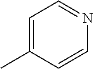 | 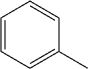 | H | 317.1 |
| 35 | 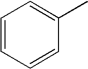 | 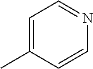 | 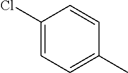 | H | 322.97/ 321.99 |
| 36 | 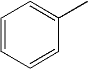 | 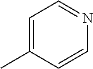 | 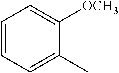 | H | 319.07 |
| 37 | 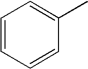 | 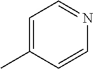 | 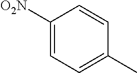 | H | 334 |
| 38 | 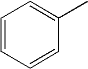 | 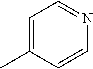 | 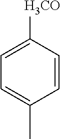 | H | 319.19 |
| 39 | 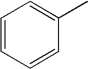 | 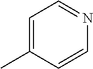 | 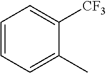 | H | 357.01 |
| 40 | 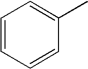 | 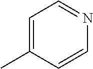 | 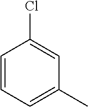 | H | 322.97/ 324.99 |
| 41 | 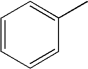 | 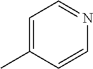 | 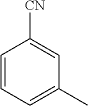 | H | 314.04 |
| 42 | 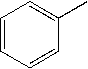 | 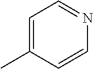 | 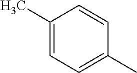 | H | 303.15 |
| 43 | 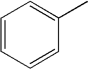 | 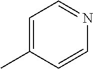 | 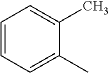 | H | 303.15 |
| 44 | 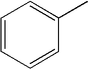 | 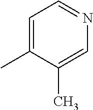 | 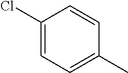 | H | 337.24/ 339.20 |
| 45 | 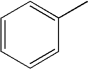 | 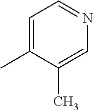 | 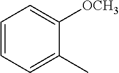 | H | 333.28 |
| 46 | 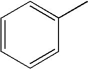 | 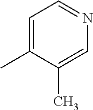 | 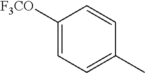 | H | 387.2 |
| 47 | 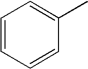 | 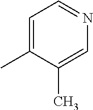 | 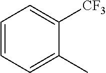 | H | 371.22 |
| 48 | 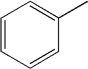 | 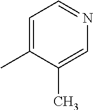 | 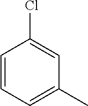 | H | 337.25/ 339.26 |
| 49 | 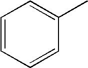 | 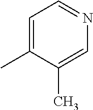 | 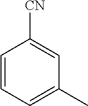 | H | 328.27 |
| 50 | 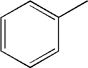 | 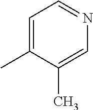 | 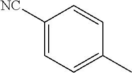 | H | 328.25 |
| 51 | 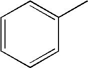 | 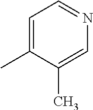 | 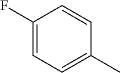 | H | 321.27 |
| 52 | 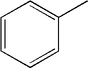 | 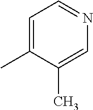 | 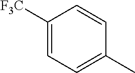 | H | 371.27 |
| 53 | 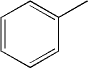 | 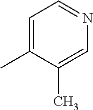 | 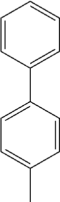 | H | 379.27 |
| 54 | 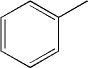 | 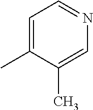 | 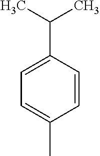 | H | 345.35 |
| 55 | 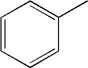 | 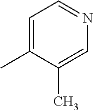 | 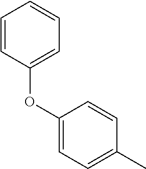 | H | 395.27 |
| 56 | 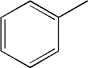 | 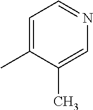 | 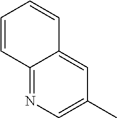 | H | 354.29 |
| 57 | 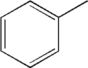 | 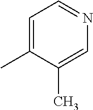 | 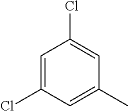 | H | 371.16/ 373.11 |
| 58 | 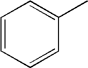 | 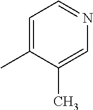 | 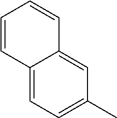 | H | 353.27 |
| 59 | 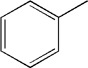 | 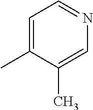 | 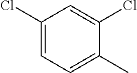 | H | 371.16/ 373.16 |
| 60 | 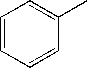 | 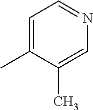 | 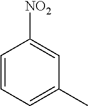 | H | 348.23 |
| 61 | 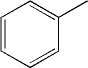 | 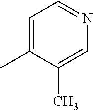 | 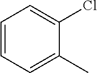 | H | 337.19 |
| 62 | 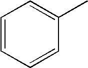 | 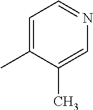 | 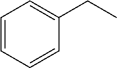 | H | 317.29 |
| 63 | 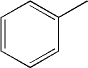 | 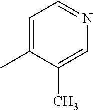 | 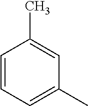 | H | 317.31 |
| 64 | 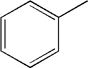 | 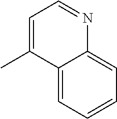 | 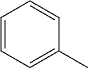 | H | 339.2 |
| 65 | 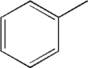 | 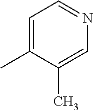 | 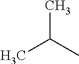 | H | 269.43 |
| 66 | 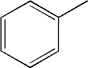 | 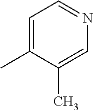 | 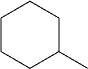 | H | 309.53 |
| 67 | 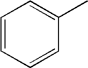 | 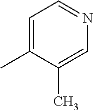 | 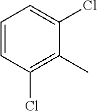 | H | 371.41/ 373.29 |
| 68 | 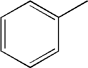 | 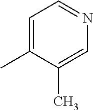 | 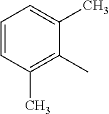 | H | 331.57 |
| 69 | 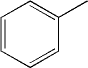 | 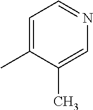 | 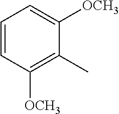 | H | 363.53 |
| 70 | 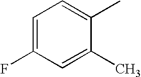 | 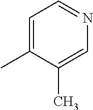 | 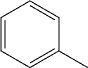 | H | 335.54 |
| 71 | 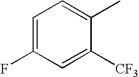 | 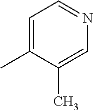 | 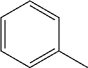 | H | 389.43 |
| 72 | 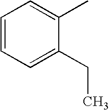 | 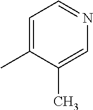 | 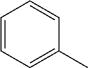 | H | 331.54 |
| 73 | 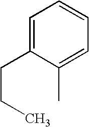 | 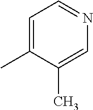 | 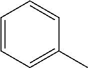 | H | 345.62 |
| 74 | 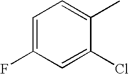 | 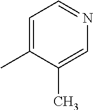 | 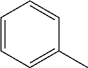 | H | 355.49 |
| 75 | 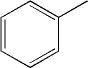 | 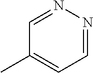 | 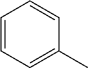 | H | 290.21 |
| 76 | 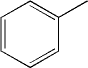 | 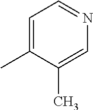 | 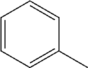 | CH₃ | 317.27 |
| 77 | 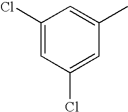 | 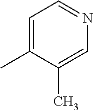 | 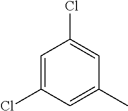 | H | 440.97/ 442.97 |
| 78 | 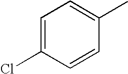 | 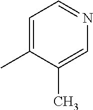 | 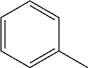 | H | 337.2 |
| 79 | 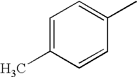 | 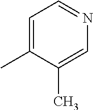 | 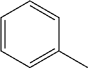 | H | 317.2 |
| 80 | 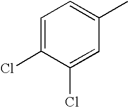 | 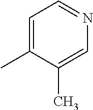 | 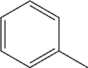 | H | 371.1 |
| 81 | 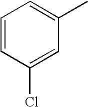 | 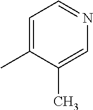 | 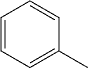 | H | 337.2 |
| 82 | 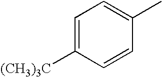 | 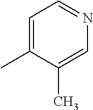 | 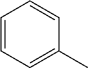 | H | 359.3 |
| 83 | 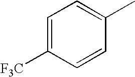 | 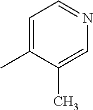 | 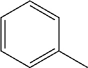 | H | 371.2 |
| 84 | 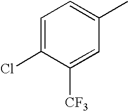 | 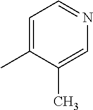 | 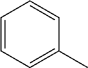 | H | 405.1 |
| 85 | 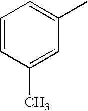 | 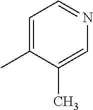 | 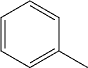 | H | 317.2 |
| 86 | 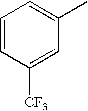 | 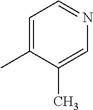 | 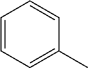 | H | 371.2 |
| 87 | 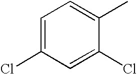 | 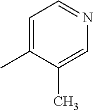 | 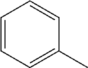 | H | 371.1 |
| 88 | 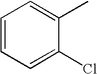 | 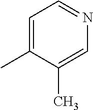 | 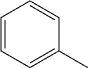 | H | 337.2 |
| 89 | 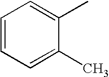 | 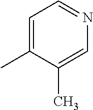 | 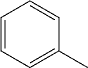 | H | 317.2 |
| 90 | 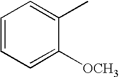 | 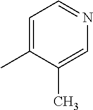 | 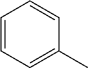 | H | 333.2 |
| 91 | 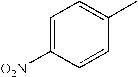 | 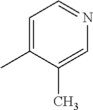 | 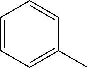 | H | 348.2 |
| 92 | 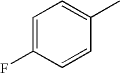 | 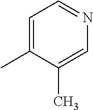 | 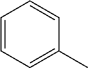 | H | 321.2 |
| 93 | 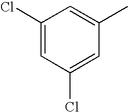 | 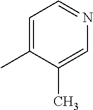 | 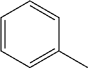 | H | 371.1 |
| 94 | 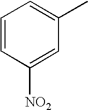 | 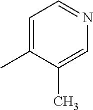 | 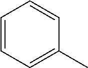 | H | 348.2 |
| 95 | 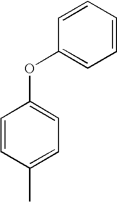 | 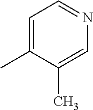 | 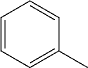 | H | 395.2 |
| 96 | 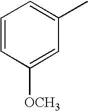 | 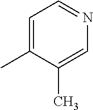 | 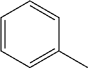 | H | 333.2 |
| 97 | 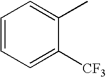 | 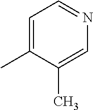 | 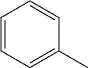 | H | 371.2 |
| 98 | 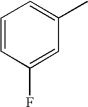 | 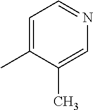 | 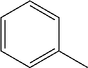 | H | 321.2 |
| 99 | 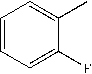 | 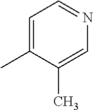 | 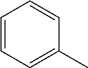 | H | 321.2 |
| 100 | 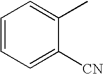 | 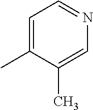 | 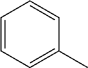 | H | 328.3 |
| 101 | 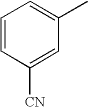 | 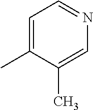 | 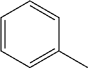 | H | 328.3 |
| 102 | 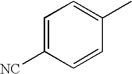 | 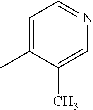 | 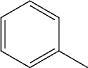 | H | 328.3 |
| 103 | 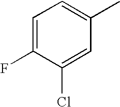 | 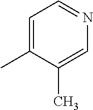 | 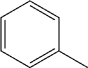 | H | 355.2 |
| 104 | 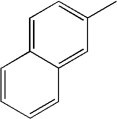 | 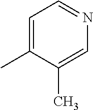 | 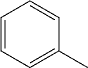 | H | 353.2 |
| 105 | 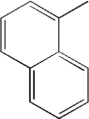 | 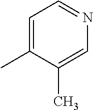 | 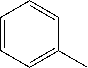 | H | 353.2 |
| 106 | 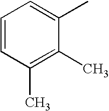 | 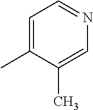 | 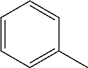 | H | 331.3 |
| 107 | 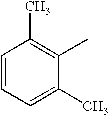 | 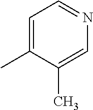 | 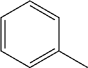 | H | 331.3 |
| 108 | 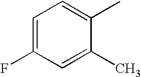 | 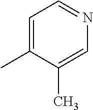 | 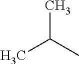 | H | 301.22 |
| 109 | 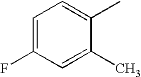 | 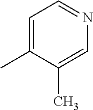 | 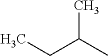 | H | 315.19 |
| 110 | 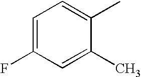 | 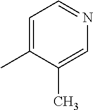 | 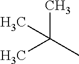 | H | 315.1 |
| 111 | 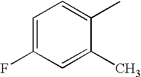 | 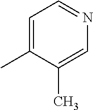 | 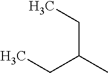 | H | 329.22 |
| 112 | 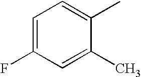 | 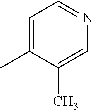 | 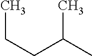 | H | 329.17 |
| 113 | 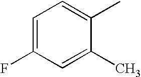 | 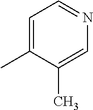 | 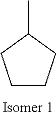 | H | 327.21 |
| 114 | 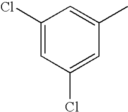 | 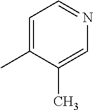 | 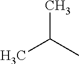 | H | 337/339 |
| 115 | 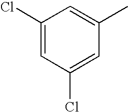 | 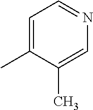 | 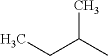 | H | 351/353 |
| 116 | 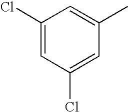 | 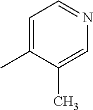 | 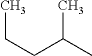 | H | 365/367 |
| 117 | 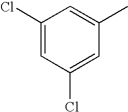 | 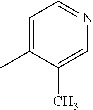 | 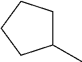 | H | 363/365 |
| 118 | 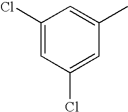 | 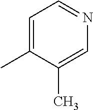 | 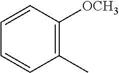 | CH₃ | 415 |
| 119 | 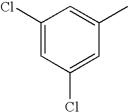 | 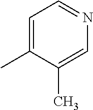 | 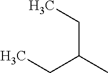 | H | 365/367 |
| 120 | 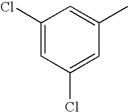 | 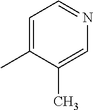 | 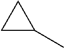 | H | 335/337 |
| 121 | 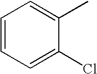 | 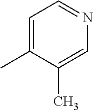 | 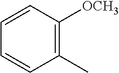 | H | 367.06/ 369.07 |
| 122 | 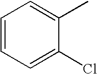 | 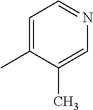 | 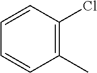 | H | 371.02/ 373.03 |
| 123 | 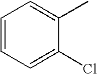 | 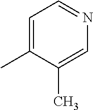 | 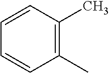 | H | 351.13/ 353.08 |
| 124 | 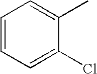 | 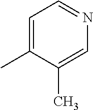 | 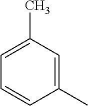 | H | 351.13/ 353.08 |
| 125 | 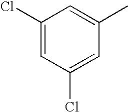 | 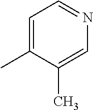 | 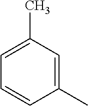 | H | 385.06/ 387.01 |
| 126 | 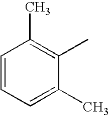 | 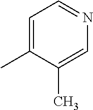 | 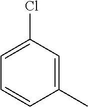 | H | 365.1/ 367.1 |
| 127 | 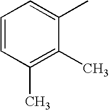 | 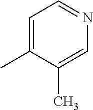 | 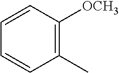 | H | 361.2 |
| 128 | 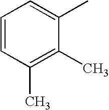 | 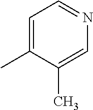 | 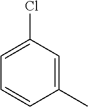 | H | 365.16/ 367.12 |
| 129 | 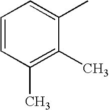 | 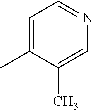 | 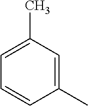 | H | 345.2 |
| 130 | 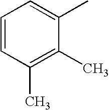 | 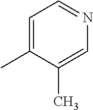 | 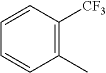 | H | 399.15 |
| 131 | 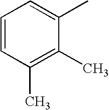 | 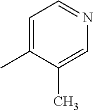 | 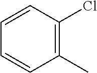 | H | 365.12/ 367.12 |
| 132 | 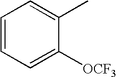 | 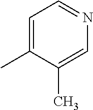 | 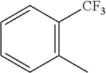 | H | 455.02 |
| 133 | 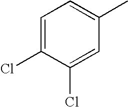 | 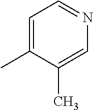 | 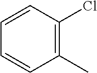 | H | 405/ 406.97 |
| 134 | 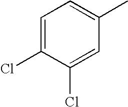 | 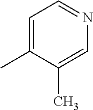 | 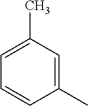 | H | 385.06/ 387.01 |
| 135 | 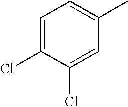 | 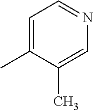 | 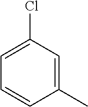 | H | 405/ 406.97 |
| 136 | 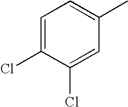 | 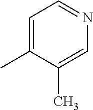 | 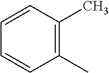 | H | 385.06/ 387.07 |
| 137 | 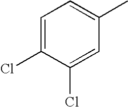 | 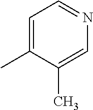 | 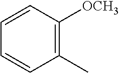 | H | 401.11/ 403.06 |
| 138 | 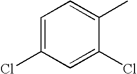 | 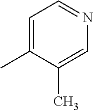 | 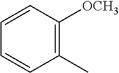 | H | 401.05/ 403.06 |
| 139 | 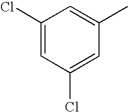 | 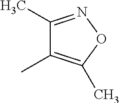 | 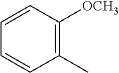 | H | 427.1/ 429.12 |
| 140 | 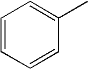 | 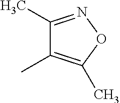 | 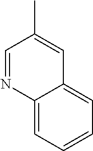 | H | 380.11 |
| 141 | 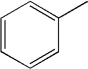 | 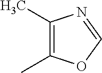 | 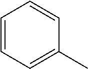 | H | 293.13 |
| 142 | 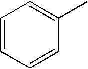 | 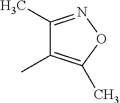 | 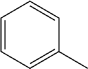 | H | 329.1 |
| 143 | 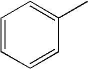 | 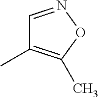 | 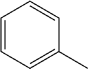 | H | 293.08 |
| 144 | 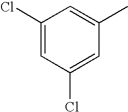 | 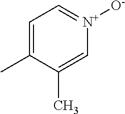 | 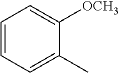 | H | 439 |
| 145 | 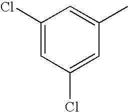 | 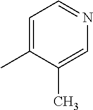 | 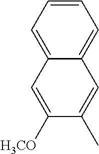 | H | 473 |
| 146 | 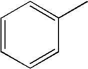 | 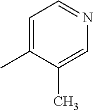 | 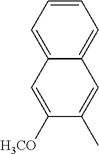 | H | 405 |
| 147 | 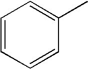 | 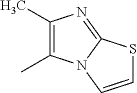 | 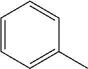 | H | 348 |
| 148 | 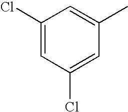 | 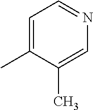 | 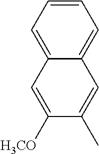 | CH₃ | 487 |
| 149 | 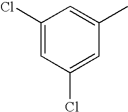 | 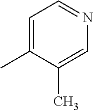 | 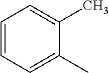 | H | 407 |
| 150 | 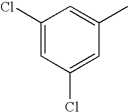 | 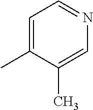 | 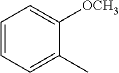 | 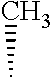 | 437 |
| 151 | 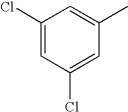 | 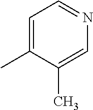 | 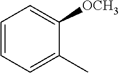 | 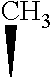 | 437 |
| 152 | 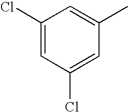 | 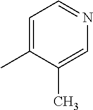 | 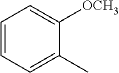 | C₂H₅ | 429 |
| 153 | 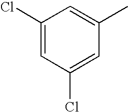 | 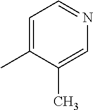 | 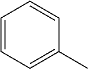 | CH₃ | 407 |
| 154 | 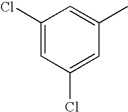 | 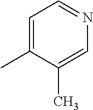 | 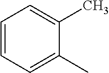 | CH₃ | 421 |
| 155 | 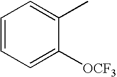 | 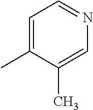 | 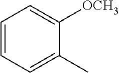 | H | 417 |
| 156 | 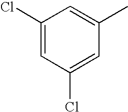 | 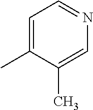 | 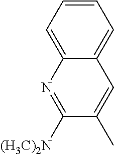 | H | 487 |
| 157 | 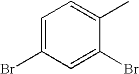 | 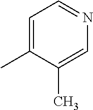 | 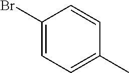 | H | 538 |
| 158 | 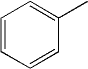 | 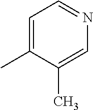 | 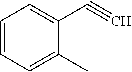 | H | 349 |
| 159 | 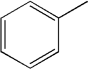 | 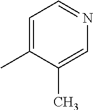 | 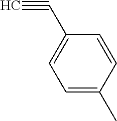 | H | 349 |
| 160 | 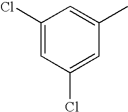 | 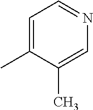 | 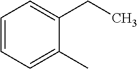 | H | 421 |
| 161 | 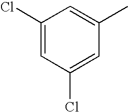 | 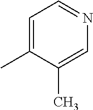 | 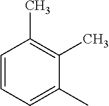 | H | 421 |
| 162 | 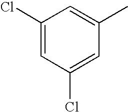 | 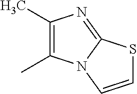 | 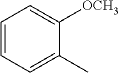 | H | 446 |
| 163 | 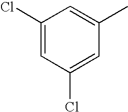 | 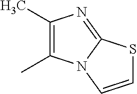 | 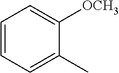 | CH₃ | 460 |
| 164 | 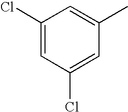 | 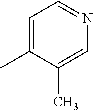 | 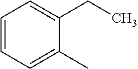 | CH₃ | 413 |
| 165 | 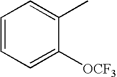 | 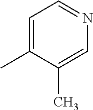 | 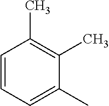 | H | 437 |
| 166 | 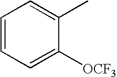 | 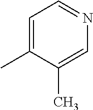 | 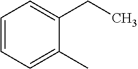 | H | 437 |
| 167 | 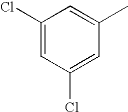 | 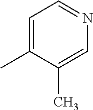 | 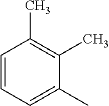 | CH₃ | 413 |
| 168 | 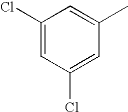 | 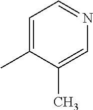 | 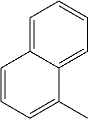 | H | 421 |
| 169 | 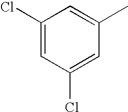 | 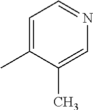 | 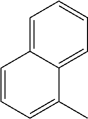 | CH₃ | 435 |
| 170 | 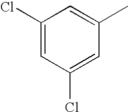 | 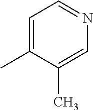 | 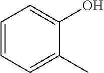 | H | 387 |
| 171 | 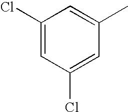 | 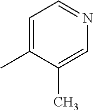 | 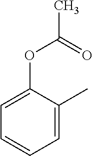 | H | 451 |
| 172 | 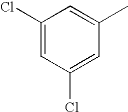 | 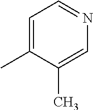 | 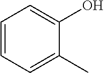 | CH₃ | 401 |
| 173 | 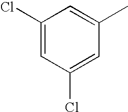 | 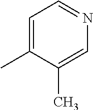 | 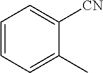 | H | 418 |
| 174 | 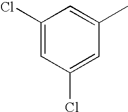 | 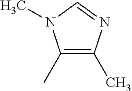 | 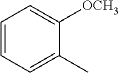 | H | 404 |
| 175 | 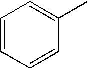 | 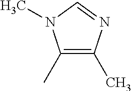 | 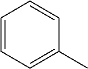 | H | 306 |
| 176 | 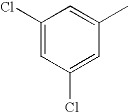 | 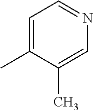 | 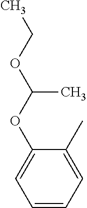 | H | 481 |
| 177 | 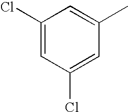 | 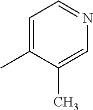 | 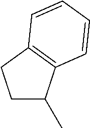 | | 419 |
| 178 | 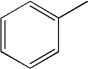 | 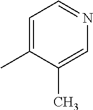 | 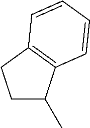 | | 351 |
| 179 | 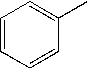 | 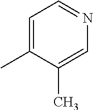 | 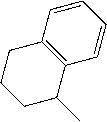 | | 365 |
| 180 | 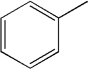 | 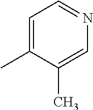 | 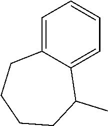 | | 379 |
| 181 | 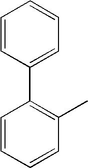 | 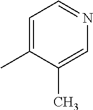 | CH₃ | H | 317 |
| 182 | 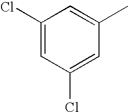 | 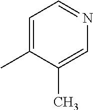 | 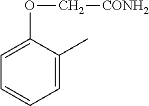 | H | 466 |
| 183 | 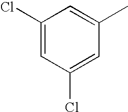 | 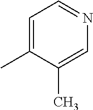 | 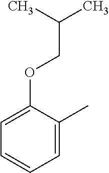 | H | 465 |
| 184 | 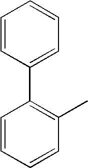 | 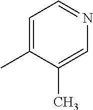 | 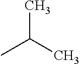 | H | 345 |
| 185 | 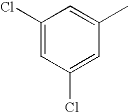 | 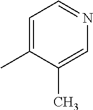 | 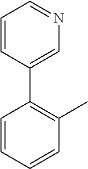 | H | 470 |
| 186 | 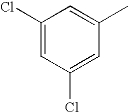 | 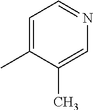 | 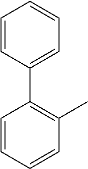 | H | 469 |
| 187 | 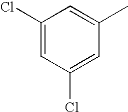 | 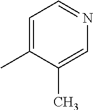 | 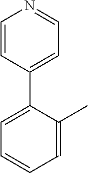 | H | 448 |
| 188 | 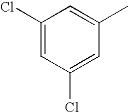 | 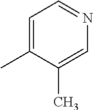 | 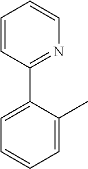 | H | 448 |
| 189 | 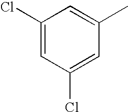 | 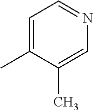 | 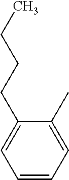 | H | 427 |
| 190 | 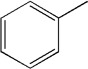 | 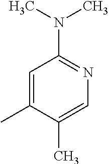 | 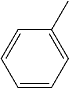 | H | 368 |
| 191 | 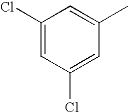 | 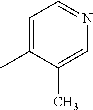 | CH₃ | H | 331 |
| 192 | 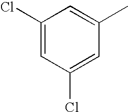 | 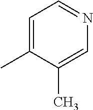 | 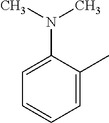 | H | 414 |
| 193 | 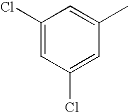 | 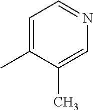 | 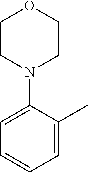 | H | 456 |
| 194 | 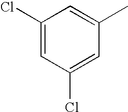 | 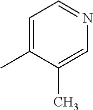 | 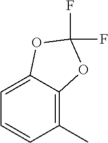 | H | 451 |
| 195 | 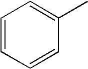 | 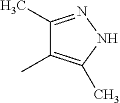 | 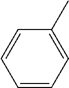 | H | 306 |
| 196 | 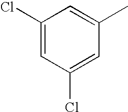 | 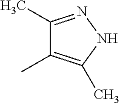 | 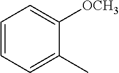 | H | 404 |
| 197 | 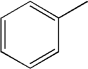 | 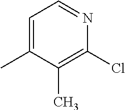 | 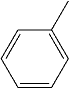 | H | 359 |
| 198 | 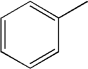 | 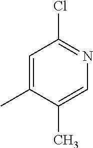 | 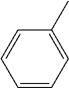 | | 359 |
| 199 | 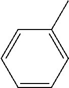 | 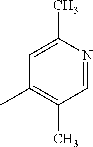 | 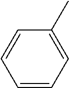 | H | 339 |
| 200 | 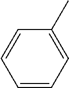 | 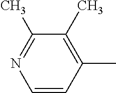 | 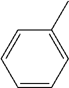 | H | 339 |
| 201 | 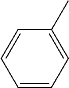 | 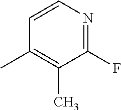 | 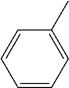 | | 343 |
| 202 | 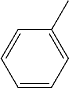 | 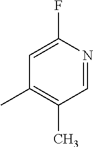 | 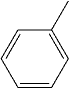 | H | 343 |
| 203 | 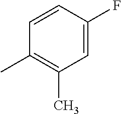 | 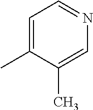 | 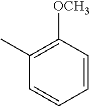 | H | 365.14 |
| 204 | 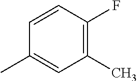 | 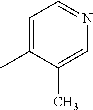 | 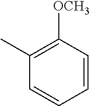 | H | 365.14 |
| 205 | 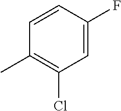 | 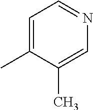 | 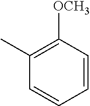 | H | 385.09 |
| 206 | 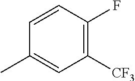 | 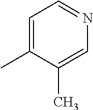 | 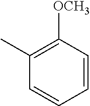 | H | 419.10 |
| 207 | 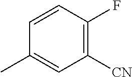 | 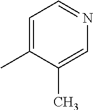 | 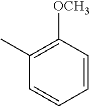 | H | 376.11 |
| 208 | 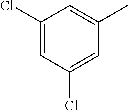 | 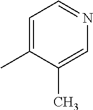 | 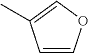 | H | 361.00 |
| 209 | 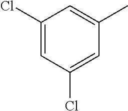 | 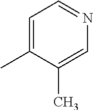 | 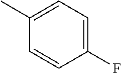 | H | 388.99 |
| 210 | 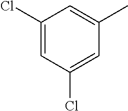 | 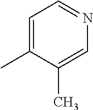 | 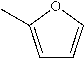 | H | 361.00 |
| 211 | 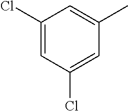 | 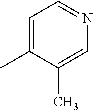 | 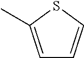 | H | 377.00 |
| 212 | 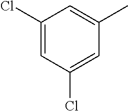 | 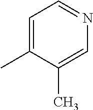 | 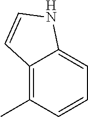 | H | 410.12 |
| 213 | 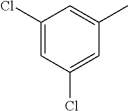 | 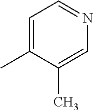 | 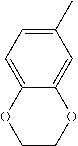 | H | 429.01 |
| 214 | 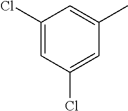 | 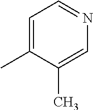 | 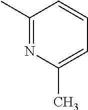 | H | 386.05 |
| 215 | 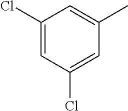 | 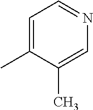 | 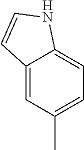 | H | 410.02 |
| 216 | 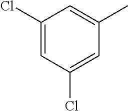 | 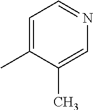 | 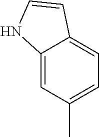 | H | 410.02 |
| 217 | 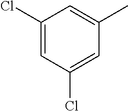 | 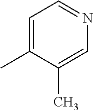 | 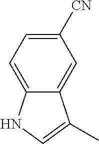 | H | 435.06 |
| 218 | 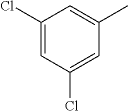 | 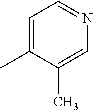 | 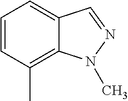 | H | 425.13 |
| 219 | 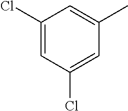 | 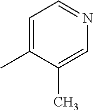 | 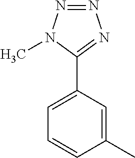 | H | 453.05 |
| 220 | 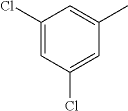 | 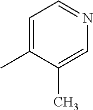 | 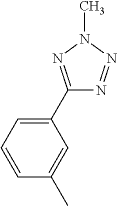 | H | 453.12 |
| 221 | 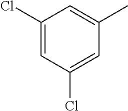 | 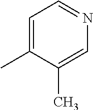 | 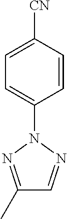 | H | 463.05 |
| 222 | 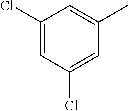 | 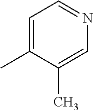 | 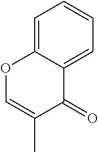 | H | 439.14 |
| 223 | 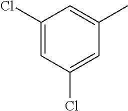 | 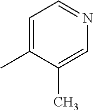 | 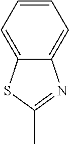 | H | 428.00 |
| 224 | 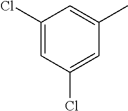 | 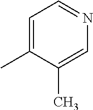 | 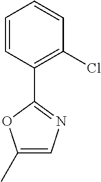 | H | 511.96 [M⁺K]⁺ |
| 225 | 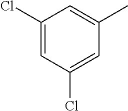 | 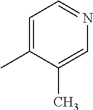 | 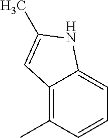 | H | 424.10 |
| 226 | 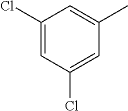 | 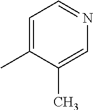 | 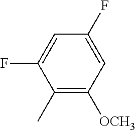 | H | 437.03 |
| 227 | 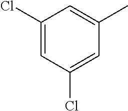 | 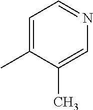 | 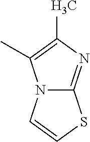 | H | 431.04 |
| 228 | 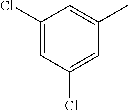 | 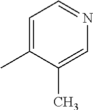 | 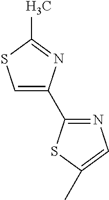 | H | 474.98 |
| 229 | 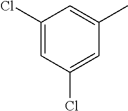 | 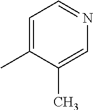 | 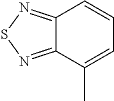 | H | |
| 230 | 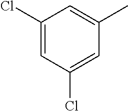 | 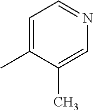 | 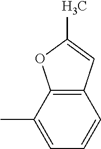 | H | 425.06 |
| 231 | 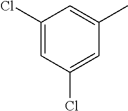 | 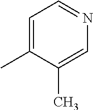 | 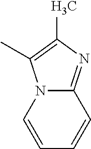 | H | 426 |
| 232 | 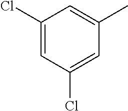 | 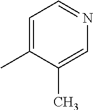 | 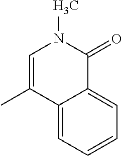 | H | 452.03 |
| 233 | 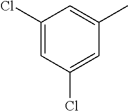 | 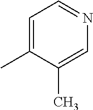 | 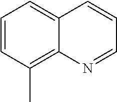 | H | 444.06 |
| 234 | 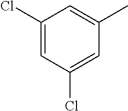 | 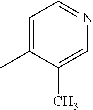 | 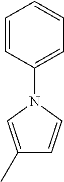 | H | |
| 235 | 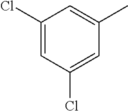 | 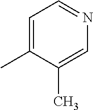 | 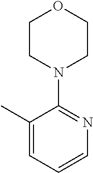 | H | 457.06 |
| 236 | 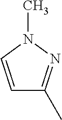 | 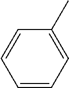 | 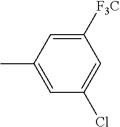 | H | 409.11 |
| 237 | 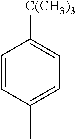 | 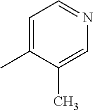 | 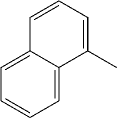 | H | 390.1 |
| 238 | 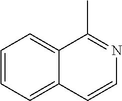 | 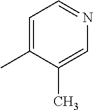 | 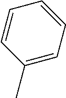 | 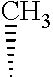 | 431.1 |
| 239 | 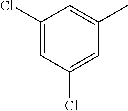 | 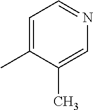 | 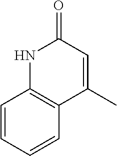 | H | 462.0 |
| 240 | 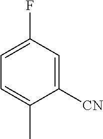 | 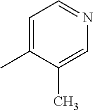 | 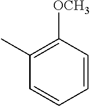 | H | 398.1 |
| 241 | 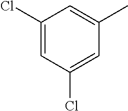 | 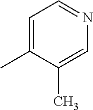 | 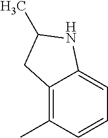 | H | |
| 242 | 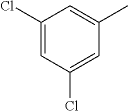 | 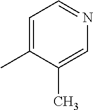 | 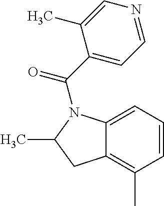 | H | |
| 243 | 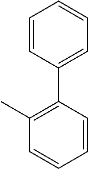 | 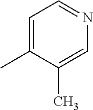 | 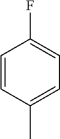 | H | 419.13 |
| 244 | 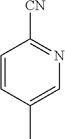 | 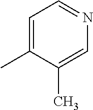 | 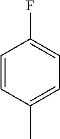 | H | 347.10 |
| 245 | 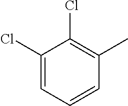 | 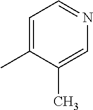 | 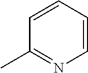 | H | 395.99 |
| 246 | 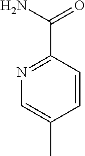 | 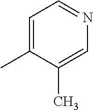 | 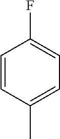 | H | 387.13 |
| 247 | 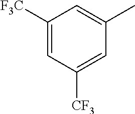 | 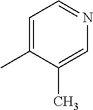 | 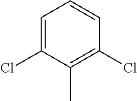 | H | 530.96 |
| 248 | 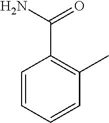 | 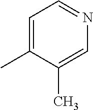 | 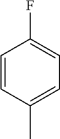 | H | 386.05 |
| 249 | 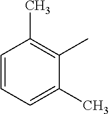 | 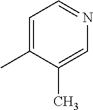 | 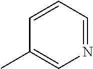 | H | 386.05 |
| 250 | 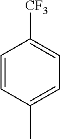 | 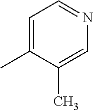 | 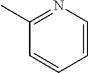 | CH₃ | 408.10 |
| 251 | 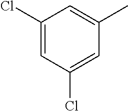 | 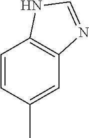 | 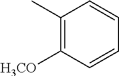 | H | 448.05 |
| 252 | 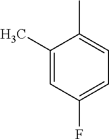 | 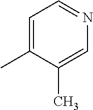 | 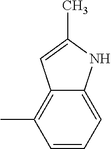 | H | 410.05 |
| 253 | 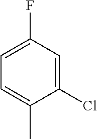 | 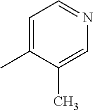 | 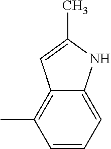 | H | 429.87 |
| 254 | 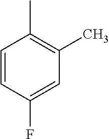 | 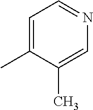 | 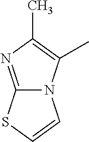 | H | 416.98 |
| 255 | 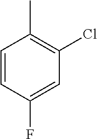 | 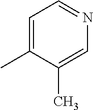 | 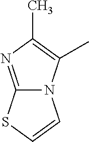 | H | 414.99 |
| 256 | 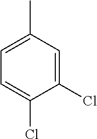 | 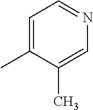 | 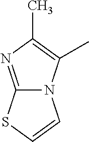 | H | 432.87 |
| 257 | 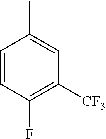 | 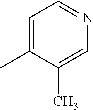 | 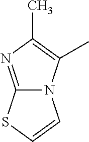 | H | 449.06 |
| 258 | 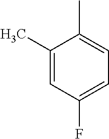 | 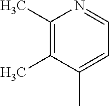 | 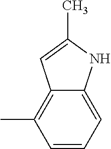 | H | 424.15 |
| 259 | 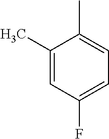 | 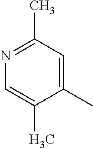 | 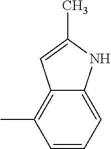 | H | 424.17 |
| 260 | 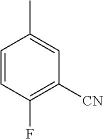 | 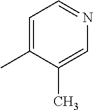 | 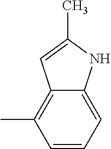 | H | 421.16 |
| 261 | 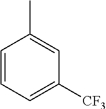 | 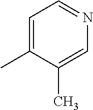 | 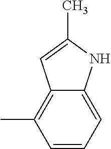 | H | 421.16 |
| 262 | 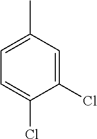 | 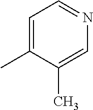 | 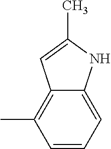 | H | 446.04 |
| 263 | 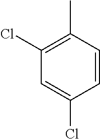 | 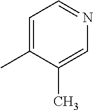 | 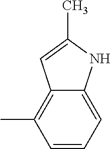 | H | 446.04 |
| 264 | 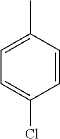 | 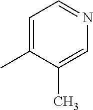 | 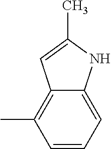 | H | 412.12 |
| 265 | 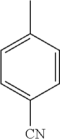 | 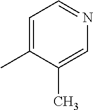 | 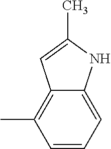 | H | 403.13 |

In TABLE 1 “EX” is the Example number and “MS” is the M⁺ peak determined in mass spectroscopy analysis. In examples 1, 2, 3, 4, 8, 140, 141, 143, 144, 145, 146, 148, 149, 150, 151, 153, 154, 156, 158, 159, 160, 161, 165, 166, 171, 173, 176, 177, 178, 179, 180, 182, 183, 185, 186, 190, 191, 197 to 202, 233, 237 to 240, 243, 245 to 254 and 258 to 265 the data set out in the column “MS” refers to the [M⁺Na]⁺-peak, in all other Examples to the [M⁺H]⁺-peak with the exception of example 224 ([M⁺K]⁺ peak).

The compound structures of the compounds indicated in TABLE 1 are confirmed by NMR spectroscopy.

1. A compound of formula 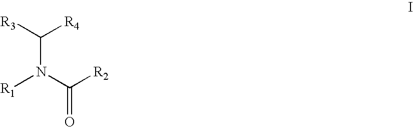
wherein
R₁ is
aryl, cyclohexyl or heterocyclyl, or (C₁₋₄)alkyl substituted by aryl, cyclohexyl or heterocyclyl,
wherein aryl is (C₆₋₁₈)aryl, and wherein aryl may be fused with aliphatic or aromatic heterocyclyl comprising 3 to 12 ring members, e.g. 6, and 1 to 4 heteroatoms selected from N, O, S,
wherein heterocyclyl is aliphatic or aromatic heterocyclyl comprising 3 to 12 ring members, and 1 to 4 heteroatoms selected from N, O, S; and wherein heterocycyl may be fused with aryl, or may be fused with another heterocyclyl comprising 3 to 12 ring members, such as 5 or 6, and 1 to 4 heteroatoms selected from N, O, S, and
wherein cycloalkyl includes (C₃₋₁₂)cycloalkyl,
R₂ is
heterocyclyl selected from the group consisting of
pyridin-4-yl, optionally in the form of an N-oxide,
Pyridin-4-yl optionally in the form of an N-oxide, substituted one or morefold by (C₁₋₄)alkyl, halo(C₁₋₄)alkyl, halogen, cyano or di(C₁₋₄alkylamino,
quinolinyl, oxazolyl, isoxazolyl, imidazo[2,1-b]thiazolyl, imidazolyl, pyrazolyl, and benzoimidazolyl,
wherein quinolinyl, oxazolyl, isoxazolyl, imidazo[2,1-b]thiazolyl, imidazolyl, pyrazolyl, or benzoimidazolyl is optionally in the form of an N-oxide and is unsubstituted or substituted one or morefold by (C₁₋₄)alkyl, halo(C₁₋₄alkyl, halogen, cyano or di(C₁₋₄alkylamino,
R₃ is
alkyl, aryl, cycloalkyl or heterocyclyl, or (C₁₋₄)alkyl substituted by aryl, cycloalkyl or heterocyclyl,
wherein alkyl includes (C₁₋₁₂)alkyl,
wherein aryl includes (C₆₋₁₈)aryl,
wherein aryl optionally is fused with aliphatic or aromatic heterocyclyl comprising 3 to 12 ring members, and 1 to 4 heteroatoms selected from N, O, S,
wherein heterocyclyl includes aliphatic or aromatic heterocyclyl comprising 3 to 12 ring members, and 1 to 4 heteroatoms selected from N, O, S and wherein heterocyclyl optionally is fused with another ring (system), and
wherein cycloalkyl includes (C₃₋₁₂)cycloalkyl,
R₄ is H or (C₁₋₄)alkyl; or
R₃ and R₄ together with the carbon atom to which they are attached are cycloalkyl, which cyclyoalkyl is fused with phenyl, such as (C₄₋₈)cycloalkyl fused with phenyl;
wherein aryl, cyclohexyl or heterocyclyl in the meaning of R₁ and R₃ is unsubstituted or one or morefold substituted by
(C₁₋₆)alkyl, (C₂₋₆)alkenyl, halo(C₁₋₄)alkyl,
oxo, hydroxy, (C₁₋₄)alkoxy, (C₁₋₄)alkoxy(C₁₋₄)alkoxy, halo(C₁₋₄alkoxy,
alkylcarbonyloxy, aminocarbonyl,
(C₆₋₁₂)aryl, (C₆₋₁₂)aryloxy,
heterocyclyl including aliphatic and aromatic heterocyclyl having 5 to 6 ring members and 1 to 4 heteroatoms selected from N, O, S, wherein heterocyclyl optionally is fused with another ring (system),
cyano, nitro, amino, di(C₁₋₄alkylamino, or halogen,
with the proviso that
compounds of formula 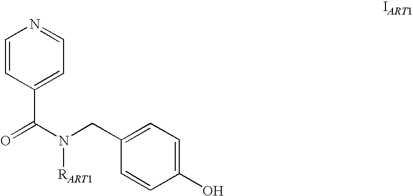
wherein R_{ART1} is selected from phenylethyl, 4-methoxyphenyl, 4-methylphenyl, isobutyl or (furan-2-yl)-methyl,
the compound N-benzyl-N-(2-naphthenyl)-isonicotinamide,
compounds of formula 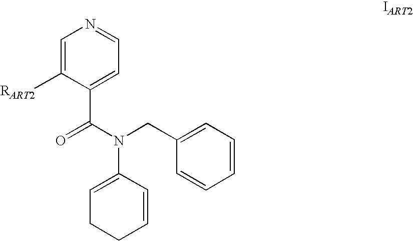
wherein R_{ART2} is iodo or benzyl, and
compounds of formula 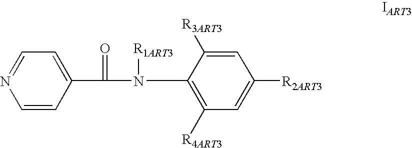
wherein R_{1ART3} is lower alkyl, R_{2ART3} is H, halogen, methyl or nitro, R_{3ART3} is H or halogen and R_{4ART3} is H, halogen, benzoyl or 3-thienoyl,
are excluded.

2. A compound according to claim 1, wherein
R₁ is
(C₆₋₁₂)aryl(C₁₋₄)alkyl, phenyl, naphthalenyl, (C₆₋₁₂)aryl substituted by (C₆₋₁₂)aryl, (C₁₋₈)alkylphenyl, di(C₁₋₄alkylphenyl, (C₁₋₄)alkoxyphenyl, halo(C₁₋₄alkyl-phenyl, bis-halo(C₁₋₄alkyl-phenyl, (halo)(halo(C₁₋₄)alkyl)phenyl, halo(C₁₋₄alkoxyphenyl, (halo)(cyano)phenyl, halophenyl, dihalophenyl, cyaonphenyl, nitrophenyl, aminocarbonylphenyl, 5 or 6-membered heterocyclyl comprising 1 to 4 heteroatoms, selected from N, O, S, or 5 or 6-membered heterocyclyl comprising 1 to 4 heteroatoms selected from N, O, S, which heterocyclyl is fused with another ring system;
R₂ is selected from the group consisting of
pyridin-4-yl, 2-methyl-pyridin-4-yl, 3-methyl-pyridin-4-yl, 2,5-dimethyl-pyridin-4-yl, 2,3-dimethyl-pyridin-4-yl, 2,5-dimethyl-pyridin-4-yl, 2-fluoro-pyridin-4-yl, 2-chloro-pyridin-4-yl, 3-chloro-pyridin-4-yl, 2-cyano-pyridin-4-yl, 3,5-dichloro-pyridin-4-yl, 2-chloro-6-methyl-pyridin-4-yl, 2-chloro-3-methyl-pyridin-4-yl, 2-chloro-5-methyl-pyridin-4-yl, 2-fluoro-3-methyl-pyridin-4-yl, 2-fluoro-5-methyl-pyridin-4-yl, 2-amino-pyridin-4-yl, 2-amino-5-methyl-pyridin, 2-dimethylamino-pyridin-4-yl, 2-dimethylamino-5-methyl-pyridin,
wherein pyridinyl is optionally in the form of an N-oxide,
quinolinyl, oxazolyl, isoxazolyl, imidazo[2,1-b]thiazolyl, imidazolyl, 1H-pyrazolyl, and benzoimidazolyl,
optionally in the form of an N-oxide,
R₃ is
alkyl substituted by (C₆₋₁₂)aryl, unsubstituted (C₁₋₁₂)alkyl,
(C₃₋₁₂)cycloalkyl, unsubstituted (C₆₋₁₂)aryl, (C₁₋₆)alkylphenyl, (C₆₋₁₂)aryl substituted by (C₆₋₁₂)aryl, di(C₁₋₄alkylphenyl, halo(C₁₋₄alkylphenyl, (C₁₋₄)alkoxyphenyl, aminocarbonyl(C₁₋₄alkoxyphenyl, (C₁₋₄)alkoxy(C₁₋₄)alkoxyphenyl, di(C₁₋₄)alkoxyphenyl, (C₆₋₁₂)aryloxyphenyl, halo(C₁₋₄alkoxyphenyl, (halo)(halo(C₁₋₄)alkyl)phenyl, (halo)((C₁₋₄)alkoxy)phenyl, cyanophenyl, hydroxyphenyl, (C₁₋₄)alkylcarbonyloxyphenyl, aminocarbonylphenyl, halophenyl, dihalophenyl, aminophenyl, di(C₁₋₄alkylaminophenyl, nitrophenyl, heterocyclylphenyl, wherein heterocyclyl comprises aromatic and aliphatic heterocyclyl, having 5 to 6 ring members and one to 4 heteroatoms selected from N, O, S, phenyl fused with heterocycyl, wherein heterocyclyl comprises 5 or 6 ring members and 1 to 4 heteroatoms, selected from N, O, S, naphthalenyl, heterocyclyl comprising 5 or 6 ring members and 1 to 4 heteroatoms selected from N, O, S, or heterocyclyl comprising 5 or 6 ring members and 1 to 4 heteroatoms selected from N, O, S, which heterocycyl is fused with another ring system,
or R₃ and R₄ together with the carbon atom to which they are attached are (C₅₋₈)cycloalkyl which cycloalkyl is fused with phenyl, and
R₄ is hydrogen or methyl,
with the proviso of claim 1.

3. A compound according to claim 1 which is selected from the group of compounds as indicated in TABLE 1 in the examples with the exception of examples 158 and 159.

4. A compound of claim 1 in the form of a salt.

5. A compound of claim 1 for use as a pharmaceutical.

6. A pharmaceutical composition comprising a compound of claim 1 in association with at least one pharmaceutical excipient.

7. A method of treating disorders mediated by GPBAR1 activity, which treatment comprises administering to a subject in need of such treatment a therapeutically effective amount of a compound of claim 1 or a compound of formula I wherein R₁ and R₂ are as defined in claim 1 and R₃ denotes (C₂₋₄)alkynyl-phenyl.

8. A compound of claim 1 or a compound of formula I wherein R₁ and R₂ are as defined in claim 1 and R₃ denotes (C₂₋₄)alkynyl-phenyl, for the manufacture of a medicament for the treatment of disorders which are mediated by GPBAR1 activity.

9. A combination of a compound of claim 1 with at least one second drug substance.

10. A compound of claim 1 or a compound of formula I wherein R₁ and R₂ are as defined in claim 1 and R₃ denotes (C₂₋₄)alkynyl-phenyl, in combination with at least one second drug substance.

11. N-(2-Ethynyl-benzyl)-3-methyl-N-phenyl-isonicotinamide, or N-(4-Ethynyl-benzyl)-3-methyl-N-phenyl-isonicotinamide,
in free form or in the form of a salt.
